(19) United States
(12) Patent Application Publication
(10) Pub. No.: US 2010/0203012 A1
(43) Pub. Date: Aug. 12, 2010

(54) IAP BIR DOMAIN BINDING COMPOUNDS

(75) Inventors: Alain Laurent, Montreal (CA); Patrick Bureau, Kirkland (CA); James Jaquith, Pincourt (CA); John Gillard, Baie d'Urfe (CA); Yannick Rose, Montreal (CA)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Aegera Therapeutics, Inc., Montreal, QC (CA)

(21) Appl. No.: 12/601,997

(22) PCT Filed: May 30, 2008
(86) PCT No.: PCT/CA2008/001041

§ 371 Date: Jan. 25, 2010

Related U.S. Application Data
(60) Provisional application No. 60/924,760, filed on May 30, 2007.

(51) Int. Cl. A61K 38/21; C07D 241/04; A61K 31/497; C12N 5/071; A61P 37/06; A61P 35/00

(52) U.S. Cl. 424/85.4; 544/357; 514/252.11; 514/255.05; 435/325

(57) ABSTRACT

Disclosed herein is a compound of Formula 1:

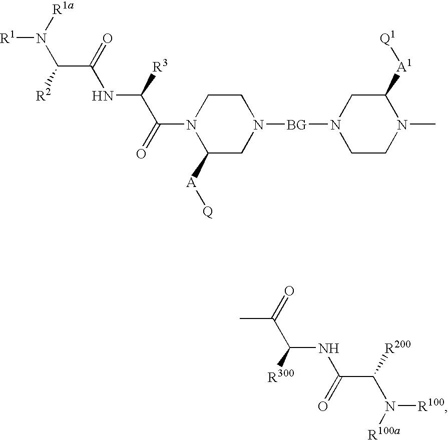

or a salt thereof, in which R¹, R^{1a}, R¹⁰⁰, R^{100a}, R², R²⁰⁰, R³, R³⁰⁰, A, A¹, Q, Q¹ and BG are as defined herein. Also disclosed is the use of the compounds of Formula 1 to treat disorders of dysregulated apoptosis, such as cancer and cellular proliferative disorders.

### FIELD OF THE INVENTION

The present invention concerns compounds that bind to IAP BIR domains and which are useful for treating disorders of dysregulated apoptosis, such as cancer and cellular proliferative disorders.

### BACKGROUND OF THE INVENTION

Apoptosis, or programmed cell death, typically occurs in the normal development and maintenance of healthy tissues in multicellular organisms. It is a complex process which results in the removal of damaged, diseased or developmentally redundant cells, in the absence of signs of inflammation or necrosis. Normal cells receive continuous feedback from their environment through various intracellular and extracellular factors, and “commit suicide” if removed from this context. This induction of apoptosis is achieved by activation of the caspase enzyme cascade, leading to an ordered proteolytic disassembly of key cellular proteins.

Progress in the cancer field has led to a new paradigm in cancer biology wherein neoplasia may be viewed as a failure of cancer cells to execute normal pathways of apoptosis. Cancer cells gain the ability to overcome or bypass this apoptosis regulation and continue with inappropriate proliferation. The majority of treatments for cancer induce at least a partial apoptotic response in the cancer target cell, resulting in remission or initiation of tumor regression. In many cases, however, residual cells which are apoptosis-resistant are capable of escaping therapy and continuing the process of oncogenic/genetic change, resulting in the emergence of highly drug-resistant, metastatic disease which overcomes our ability to effectively treat the disease. Chemoresistance results from numerous mechanisms, including alterations in the apoptotic machinery due to increased activity of anti-apoptotic pathways or expression of anti-apoptotic genes.

Furthermore, most cancer therapies cause additional cellular injury, due to their lack of specificity in inducing apoptosis solely in cancer cells. The need to improve the specificity/potency of pro-apoptosis agents used to treat cancer, and indeed other proliferative disorders, is important because of the benefits in decreasing the side effects associated with administration of these agents. Therefore, finding novel means of inducing apoptosis in cancer cells is a highly desired medical need and its solution offers the possibility of entirely new treatments for cancer.

Intrinsic apoptotic pathways are also known to be dysregulated in certain autoimmune disorders such as multiple sclerosis wherein effector T cells are resistant to normal apoptotic cues and in inflammation, in which immune or effector cells survive and in fact proliferate in the presence normally lethal cytokines, such as Tumor Necrosis Factor (TNF).

The caspase family of proteolytic enzymes, which are known to initiate and execute apoptosis, is from the class of cysteine proteases. In normal cells, the caspases are present as inactive zymogens, which are catalytically activated following external signals, for example those resulting from ligand driven Death Receptor activation, such as cytokines or immunological agents, or by release of mitochondrial factors, such as cytochrome C following genotoxic, chemotoxic, or radiation-induced cellular injury.

The Inhibitors of Apoptosis Proteins (IAPs) constitute a family of proteins which are capable of binding to and inhibiting the caspases, thereby suppressing cellular apoptosis (1-3). Because of their central role in regulating caspase activity, the IAPs are capable of inhibiting programmed cell death from a wide variety of triggers, which include loss of homeostatic, or endogenous cellular growth control mechanisms, as well as chemotherapeutic drugs and irradiation. Therefore, the IAPs are key regulators of both caspase activity and cellular survival.

The IAPs contain one to three homologous structural domains known as baculovirus IAP repeat (BIR) domains. They may also contain a RING zinc finger domain at the C-terminus, with a capability of inducing ubiquitinylation of IAP-binding molecules via its E3 ligase function. The human IAPs, XIAP, HIAP1 (also referred to as cIAP2), and HIAP2 (cIAP1) each have three BIR domains, and a carboxy terminal RING zinc finger. Another IAP, NAIP, has three BIR domains (BIR1, BIR2 and BIR3), but no RING domain, whereas Livin, TsIAP and MLIAP have a single BIR domain and a RING domain. The X chromosome-linked inhibitor of apoptosis (XIAP) is an example of an IAP which can inhibit the initiator caspase, known as caspase-9, and the effector caspases, Caspase-3 and Caspase-7, by direct binding. It can also induce the removal of caspases through the ubiquitylation-mediated proteasome pathway via the E3 ligase activity of a RING zinc finger domain. It is via the BIR3 domain that XIAP binds to and inhibits caspase-9. The linker-BIR2 domain of XIAP inhibits the activity of caspases-3 and -7. The BIR domains have also been associated with the interactions of IAPs with tumor necrosis factor-receptor associated factor (TRAFs)-1 and -2, and to TAB1, as adaptor proteins effecting survival signaling through NFkB activation. The IAPs thus function as a direct brake on the apoptosis cascade, by preventing the action of, or inhibiting active caspases and by re-directing cellular signaling to a pro-survival mode.

A growing body of data indicates that cancer cells avoid apoptosis by the sustained over-expression of one or more members of the IAP family of proteins, as documented in many primary tumor biopsy samples, as well as most established cancer cell lines. Further, epidemiological studies have demonstrated that over-expression of the various IAPs is associated with poor clinical prognosis and survival (4-8). For XIAP this is shown in cancers as diverse as leukemia and ovarian cancer. Over expression of cIAP1 (HAIP2) and cIAP2 (HAIP1) resulting from the frequent chromosome amplification of the 11q21-q23 region, which encompasses both IAP genes has been observed in a variety of malignancies, including medulloblastomas, renal cell carcinomas, glioblastomas, and gastric carcinomas. (X)IAP negative regulatory molecules such as XAF, appear to be tumor suppressors, which are very frequently lost in clinical cancers. Thus, by their ability to suppress the activation and execution of the intrinsic mediators of apoptosis, the caspases, the IAPs may directly contribute to tumor progression and resistance to pharmaceutical intervention. Decreased IAP expression through RNA antisense or siRNA strategies sensitizes tumor cells to a wide variety of apoptotic insults including chemotherapy, radiotherapy and death receptor ligands (9-22). Thus, on the basis of their over-expression in cancer and the role they play in protecting cancer cells from the induction of apoptosis, IAPs are valid targets in cancer therapy.

This invention describes novel, potent small molecules which bind to BIR domains of each of the IAPs and antagonize their function. Based upon the evidence above and herein, compounds of formula 1 will have use in the treatment of various cancers.

Apoptosis also plays a central role in the development, maintenance and function of the immune system. Levels of T cell apoptosis influence the immune repertoire and contribute to the maintenance of homeostasis. This has implications for autoimmune disease.

Abnormally apoptotic resistant T-cells have been demonstrated in autoimmune diseases such as multiple sclerosis, rheumatoid arthritis, idiopathic thrombocytopenic purpura, and alopecia areata. This suggests a potential pathogenic mechanism, resulting from the defective apoptotic termination of autoreactive T lymphocytes (23), which is supported by a number of studies which suggests that pathogenic autoreactive T-lymphocytes may escape regular apoptotic control by the upregulation of the IAPs (23, 26-30). We have demonstrated that the administration of XIAP anti-sense induces apoptotic death in antigen-activated T cells and prevents the progression of MS like symptoms in an EAE mouse model of MS (31).

Thus, therapies which induce apoptosis of autoreactive T-lymphocytes by blocking the function of the IAPs represent novel approaches for treating autoimmune diseases such as MS. Additionally, other autoimmune or inflammatory diseases are characterized by the presence of such abnormally apoptosis-resistant cells such as fibroblast-like synoviocytes in rheumatoid arthritis (29). Recently, it has been observed that these potentially pathogenic cells also over-express individual IAPs.

Compounds of the present invention target the IAPs, resulting in their antagonism or loss of function in many cell-types and may as well, potently induce apoptosis of non-cancerous rheumatoid synoviocytes and T lymphocytes. Hence, small molecules as described herein, which target the IAPs, will also be useful in the treatment of a variety of non-cancerous proliferative disorders, including inflammatory disorders and auto-immune diseases, wherein failure of pathogenic cells to apoptose is a contributing factor

We and others have demonstrated the critical importance of the individual BIR domains for affecting the antiapoptotic function of the IAPs. Indeed, individual BIRs serve as critical binding sites for the N-terminal Ser-Gly-Val-Asp, Ser-Gly-Pro-Ile and Ala-Thre-Pro-Phe residues of the Caspases 3, 7, and 9, respectively, and such binding is imperative for the Caspase-inhibitory function of the IAPs. We have proposed that agents which may bind to the individual BIR domains, may disrupt the antiapoptotic function of the IAPs.

The overall topology of the related BIR domains is highly conserved between the human IAPs and within an individual IAP, each BIR containing a RING zinc finger polypeptide domain. The X-ray crystallographic structures of XIAP BIR2 and BIR3 reveal the critical binding pocket for an AXPY motif on the surface of both BIR domains. There are differences in the intervening amino acid sequences that form the binding pockets of both BIR2 and BIR3. Likewise, cIAP1 and cIAP2 contain slightly different intervening amino acid sequences in their homologous BIR domains.

A mammalian mitochondrial protein, namely Second Mitochondria-derived Activator of Caspases (SMAC) which antagonizes IAP function, binds mainly to the BIR 3 or BIR 2 domain on respective IAPs via the N-terminal tetrapeptide AVPI. Four Drosophila death-inducing proteins, Reaper, HID, Grim, and Sickle, which antagonize the ability of the Drosophila IAPs to inhibit caspases, also bind the BIR domains of the analogous Drosophila IAPs via their N-terminal tetrapeptide sequence which defines a generic tetra-peptide sequence Ala-(X)-Pro-(Y)- that fits into the BIR binding pocket and disrupts IAP-caspase interactions.

The binding of an isolated Ala-(Thre)-Pro-(Ile) N-terminal tetra-peptide motif to BIR3 of XIAP results in the release of the active caspases 9 and 3, 7. In the case of other IAPs, such as c-IAP1 and c-IAP2, the binding of Ala-X-Pro-Y tetra-peptide motifs appear to direct the activation of the E3 ubiquitin ligase RING function of said IAPs to a bound target, or individual IAPs themselves, leading to proteosomal loss.

A number of compounds have been reported to bind to IAPs. For a recent review see Elmore et al., Annual Reports in Medicinal Chemistry, 40 (2006) 245-262. Also see Vince, J. E., et al. Cell (2007) 131, 682-693; Sun, H.; et al. J. Am. Chem. Soc. (2007) 129, 15279-15294. Varfolomeev, E., et al. Cell (2007) 131, 669-681; Petersen, S. L., et al. Cancer Cell (2007) 12, 445-456; Sun et al., Bioorg. Med. Chem. Let. 15 (2005) 793-797; Oost et al., J. Med. Chem., 2004, 47(18), 4417-4426; Park et al., Bioorg. Med. Chem. Lett. 15 (2005) 771-775; Franklin et al., Biochemistry, Vol. 42, No. 27, 2003, 8223-8231; Kip et al., Biochemistry 2002, 41, 7344-7349; Wu et al., Chemistry and Biology, Vol. 10, 759-767 (2003); Glover et al., Analytical Biochemistry, 320 (2003) 157-169; United States published patent application number 20020177557; and United States published patent application number 20040180828; United States published patent application number US2006/0025347A1; United States published patent application number US2005/0197403A1; United States published patent application number US2006/0194741A1; WO 2005/094818 A1; PCT/US2006/0014700 A1: WO 2006/069063 A1; WO 2007/106192; WO 2004/005248 A1; WO 2005/097791 A1; PCT/US2006/048163; WO 2006/113376 A1; WO 2006/133147 A2; WO 2008/016893 A1; WO 2008/016893 A1; WO 2005/084317 A2; WO 2007/136921 A2; United States published patent application number US 2006/0025347 A1; WO 2008/014252 A2; WO 2008/014263 A2; WO 2008/014238 A2; WO 2008/014229 A2; WO 2008/014240 A2; WO 2008/014236 A1; and United States published patent application number US 2008/0089896 A1.

These reported IAP binding compounds have been shown to target an isolated BIR3 domain of XIAP via displacement of a fluorescently-labeled probe and they appear to induce an apoptotic event in a select set of cancer cell lines with potency in the low micromolar-nanomolar range.

It is our finding that the novel class of compounds as described herein, binds to IAP BIRs with exceptional potency, resulting in both loss of XIAP function and loss of cellular IAP protein, conveying a distinct therapeutic advantage for the treatment of cancer either as a single agent, or in combination with chemotherapeutic agents and in particular, agonists of the TNF receptor superfamily such as TRAIL and TRAIL receptor monoclonal antibodies.

The inventors have previously disclosed a series of compounds which bind to the BIR domains of the IAPs and induce apoptosis in various cancer cell lines (US published patent application number 20060264379). A characteristic of these compounds is the presence of a central pyrrolidine unit. The applicants have also disclosed that the bridging of two BIR binding units, with preference for the site, orientation and chemical nature of the bridge, provides novel and distinctly advantageous classes of compounds with up to, for example, 1000 fold increase in potency over their corresponding non-bridged BIR binding compounds, as measured by the induction of apoptosis against various cancer cell lines (see US patent application numbers US20070093428; US20080069812 A1; PCT/CA2007/000887). Additionally, these compounds display the requisite potency, stability and pharmaceutical properties for the treatment of human cancers in vivo. Thus, IAP BIR domains represent an attractive target for the discovery and development of novel therapeutic agents, especially for the treatment of proliferative disorders such as cancer, autoimmune and inflammatory diseases.

### SUMMARY OF THE INVENTION

The inventors have previously disclosed a series of compounds which bind to the BIR domains of the IAPs and induce apoptosis in various cancer cell lines (US published patent application number 20060264379). A characteristic of these compounds is the presence of a central pyrrolidine unit. We have now discovered a novel class of compounds in which two BIR binding units are bridged via a substituted piperazine unit. The compounds demonstrate a significant increase in potency against various cancer cell lines compared to their corresponding non-bridged BIR binding compounds.

In one embodiment of the present invention, there is provided a compound represented by Formula 1:

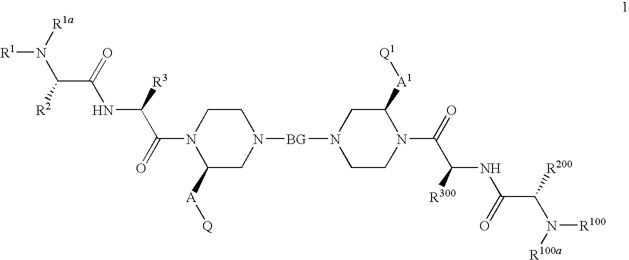

or a salt thereof,
wherein
n is 0 or 1;
m is 0, 1 or 2;

### Y is NR⁸, O or S;

### BG is

1) —X-L-X¹—; or
2) L;
   X and X¹ are independently selected from

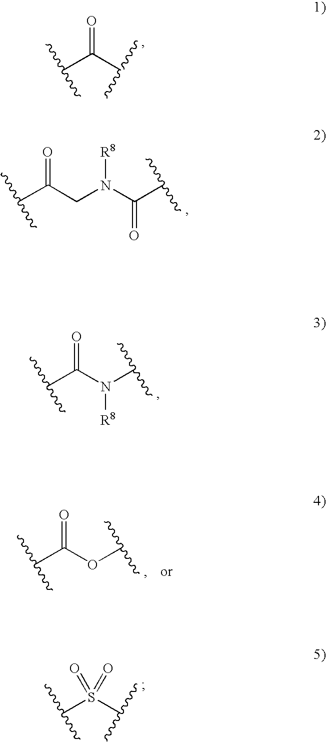

L is selected from:
1) —C₁-C₂₀ alkyl-,
2) —C₂-C₆ alkenyl-,
3) —C₂-C₄ alkynyl-,
4) —C₃-C₇ cycloalkyl-,
5) -aryl-,
6) -biphenyl-,
7) -heteroaryl-,
8) -heterocyclyl-,
9) —C₁-C₆ alkyl-(C₂-C₆ alkenyl)-C₁-C₆ alkyl-,
10) —C₁-C₆ alkyl(C₂-C₄ alkynyl)-C₁-C₆ alkyl-
11) —C₁-C₆ alkyl(C₃-C₇ cycloalkyl)-C₁-C₆ alkyl-,
12) —C₁-C₆ alkyl-aryl-C₁-C₆ alkyl-,
13) —C₁-C₆ alkyl-biphenyl-C₁-C₆ alkyl-,
14) —C₁-C₆ alkyl-heteroaryl-C₁-C₆ alkyl-,
15) —C₁-C₆ alkyl-heterocycyl-C₁-C₆ alkyl-,
16) —C₁-C₆ alkyl-Y—C₁-C₆ alkyl-,
17) -aryl-Y-aryl-,
18) —C₁-C₆ alkyl-Z—C₁-C₆ alkyl-, or
19) -aryl-Z-aryl-.
wherein the alkyl, alkenyl, alkynyl and cycloalkyl are optionally substituted with one or more R⁶ substituents, and the aryl, biphenyl, heteroaryl, and heterocyclyl are optionally substituted with one or more R¹⁰ substituents;
   Z is selected from:
1) —C(O)—,
2) —S(O)₂—,
3) —N(R⁸)C(O)—,
4) —C(O)N(R⁸)—,
5) —C(O)N(R⁸)—,
6) —S(O)₂N(R⁸)—,
7) —N(R⁸)—C(O)—N(R⁸)—,
8) —N(R⁸)—C(O)C(O)—N(R⁸)—,
9) —N(R⁸)—C(O)—C₁-C₁₂-alkyl-C(O)—N(R⁸)—,
10) —N(R⁸)—C(O)-aryl-C(O)—N(R⁸)—,
11) —N(R⁸)—C(O)-aryl-O-aryl-C(O)—N(R⁸)—,
12) —N(R⁸)—C(O)-heteroaryl-C(O)—N(R⁸)—,
13) —N(R⁸)—C(O)-biphenyl-C(O)—N(R⁸)—,
14) —N(R⁸)—S(O)₂—C₁-C₁₂-alkyl-S(O)₂—N(R⁸)—,
15) —N(R⁸)—S(O)₂-aryl-S(O)₂—N(R⁸)—,
16) —N(R⁸)—S(O)₂-heteroaryl-S(O)₂—N(R⁸)—,
17) —N(R⁸)—S(O)₂-biphenyl-S(O)₂—N(R⁸)—,
18) —N(R⁸)—C₁-C₁₂-alkyl-N(R⁸)—,
19) —N(R⁸)-aryl-N(R⁸)—,
20) —N(R⁸)-heteroaryl-N(R⁸)—, or
21) —N(R⁸)-biphenyl-N(R⁸)—;
   wherein the alkyl is optionally substituted with one or more R⁶ substituents, and the aryl, the heteroaryl, the biphenyl and the heterocyclyl are optionally substituted with one or more R¹⁰ substituents;
   Q and Q¹ are independently selected from
1) —NR⁴R⁵,
2) —OR¹¹,
3) —S(O)ₘR¹¹; or
   Q and Q¹ are independently selected from
1) aryl, or
2) heteroaryl.
   wherein the aryl and the heteroaryl are optionally substituted with one or more R¹⁰ substituents;
   A and A¹ are independently selected from
1) —CH₂—,
2) —CH₂CH₂—,
3) —CH(C₁-C₆ alkyl)-,
4) —CH(C₃-C₇ cycloalkyl)-,
5) —C₃-C₇ cycloalkyl-,
6) —CH(C₁-C₆ alkyl-C₃-C₇ cycloalkyl)-,
7) —C(O)—, or
   wherein the alkyl and cycloalkyl are optionally substituted with one or more R⁶ substituents;
   R¹, R^{1a}, R¹⁰⁰, and R^{100a} are independently selected from
1) H, or
2) C₁-C₆ alkyl optionally substituted with one or more R⁶ substituents;
   R² and R²⁰⁰ are independently selected from
1) H, or
2) C₁-C₆ alkyl optionally substituted with one or more R⁶ substituents;
   R³ and R³⁰⁰ are independently selected from
1) H,
2) C₁-C₆ alkyl,
3) C₃-C₇ cycloalkyl,
4) C₃-C₇ cycloalkenyl,
5) aryl,
6) biphenyl,
7) heteroaryl,
8) heterocyclyl,
9) heterobicyclyl,
   wherein the alkyl, cycloalkyl and cycloalkenyl are optionally substituted with one or more R⁶ substituents; and wherein the aryl, biphenyl, heteroaryl, heterocyclyl and heterobicyclyl are optionally substituted with one or more R¹⁰ substituents.
   R⁴ and R⁵ are each independently selected from
1) H,
2) haloalkyl,
3) C₁-C₆ alkyl,
4) C₂-C₆ alkenyl,
5) C₂-C₄ alkynyl,
6) C₃-C₇ cycloalkyl,
7) C₃-C₇ cycloalkenyl,
8) aryl,
9) biphenyl,
10) heteroaryl,
11) heterocyclyl,
12) heterobicyclyl,
13) aryl-heteroaryl,
14) heteroaryl-aryl,
15) heterocyclyl-aryl,
16) —C(O)ₙ—R¹¹,
17) —S(O)₂—R¹¹, or
18) —C(═Y)NR⁸R⁹,
   wherein the alkyl, the alkenyl, the alkynyl, the cycloalkyl and the cycloalkenyl are optionally substituted with one or more R⁶ substituents, and the aryl, the biphenyl, the heteroaryl, the heterocyclyl and the heterobicyclyl are optionally substituted with one or more R¹⁰ substituents

### R⁶ is

1) halogen,
2) NO₂,
3) CN,
4) haloalkyl,
5) C₁-C₆ alkyl,
6) C₂-C₆ alkenyl,
7) C₂-C₄ alkynyl,
8) C₃-C₇ cycloalkyl,
9) C₃-C₇ cycloalkenyl,
10) aryl,
11) heteroaryl,
12) heterocyclyl,
13) heterobicyclyl,
14) —OR⁷,
15) —S(O)ₘR⁷,
16) —NR⁸R⁹,
17) —NR⁸S(O)₂R¹¹,
18) —C(O)OₙR⁷,
19) —C(O)NR⁸R⁹,
20) —S(O)₂NR⁸R⁹
21) —OC(O)R⁷,
22) —OC(O)Y—R¹¹,
23) —SC(O)R⁷, or
24) —NC(Y)NR⁸R⁹.
   wherein the aryl, heteroaryl, heterocyclyl and heterobicyclyl is optionally substituted with one or more R¹⁰ substituents

### R⁷ is

1) H,
2) haloalkyl,
3) C₁-C₆ alkyl,
4) C₂-C₆ alkenyl,
5) C₂-C₄ alkynyl,
6) C₃-C₇ cycloalkyl,
7) C₃-C₇ cycloalkenyl,
8) aryl,
9) biphenyl,
10) heteroaryl,
11) heterocyclyl,
12) heterobicyclyl,
13) —C₁-C₆ alkyl-C₂-C₄ alkenyl, or
14) —C₁-C₆ alkyl-C₂-C₄ alkynyl,
   wherein the alkyl, alkenyl, alkynyl, cycloalkyl and cycloalkenyl are optionally substituted with one or more R⁶ substituents; and wherein the aryl, biphenyl, heteroaryl, heterocyclyl and heterobicyclyl are optionally substituted with one or more R¹⁰ substituents;
   R⁸ and R⁹ are each independently
1) H,
2) -haloalkyl,
3) —C₁-C₆ alkyl,
4) —C₂-C₆ alkenyl,
5) —C₂-C₄ alkynyl,
6) —C₃-C₇ cycloalkyl,
7) —C₃-C₇ cycloalkenyl,
8) aryl,
9) biphenyl,
10) heteroaryl,
11) heterocyclyl,
12) heterobicyclyl,
   wherein the alkyl, alkenyl, alkynyl, cycloalkyl and cycloalkenyl are optionally substituted with one or more R⁶ substituents; and wherein the aryl, biphenyl, heteroaryl, heterocyclyl and heterobicyclyl are optionally substituted with one or more R¹⁰ substituents;
   or R⁸ and R⁹ together with the nitrogen atom to which they are bonded form a five, six or seven membered heterocyclic ring optionally substituted with one or more R⁶ substituents;

### R¹⁰ is

1) halogen,
2) NO₂,
3) CN,
4) —B(OR¹³)(OR¹⁴),
5) —C₁-C₆ alkyl,
6) —C₂-C₆ alkenyl,
7) —C₂-C₄ alkynyl,
8) —C₃-C₇ cycloalkyl,
9) —C₃-C₇ cycloalkenyl,
10) haloalkyl,
11) —OR⁷,
12) —NR⁸R⁹,
13) —SR⁷,
14) —COR⁷,
15) —C(O)OR⁷,
16) —S(O)ₘR⁷,
17) —CONR⁸R⁹,
18) —S(O)₂NR⁸R⁹,
19) aryl,
20) biphenyl,
21) heteroaryl,
22) heterocyclyl, or
23) heterobicyclyl,
   wherein the alkyl, alkenyl, alkynyl, cycloalkyl and cycloalkenyl are optionally substituted with one or more R⁶ substituents;

### R¹¹ is

1) haloalkyl,
2) C₁-C₆ alkyl,
3) C₂-C₆ alkenyl,
4) C₂-C₄ alkynyl,
5) C₃-C₇ cycloalkyl,
6) C₃-C₇ cycloalkenyl,
7) aryl,
8) biphenyl,
9) heteroaryl,
10) heterocyclyl, or
11) heterobicyclyl,
   wherein the alkyl, alkenyl, alkynyl, cycloalkyl and cycloalkenyl are optionally substituted with one or more R⁶ substituents; and wherein the aryl, biphenyl, heteroaryl, heterocyclyl and heterobicyclyl are optionally substituted with one or more R¹⁰ substituents;

### R¹² is

1) haloalkyl,
2) C₁-C₆ alkyl,
3) C₂-C₆ alkenyl,
4) C₂-C₄ alkynyl,
5) C₃-C₇ cycloalkyl,
6) C₃-C₇ cycloalkenyl,
7) aryl,
8) biphenyl,
9) heteroaryl,
10) heterocyclyl,
11) heterobicyclyl,
12) —C(O)—R¹¹,
13) —C(O)O—R¹¹,
14) —C(O)NR⁸R⁹,
15) —S(O)ₘ—R¹¹, or
16) —C(═Y)NR⁸R⁹,
   wherein the alkyl, alkenyl, alkynyl, cycloalkyl and cycloalkenyl are optionally substituted with one or more R⁶ substituents; and wherein the aryl, biphenyl, heteroaryl, heterocyclyl and heterobicyclyl are optionally substituted with one or more R¹⁰ substituents;
   R¹³ and R¹⁴ are optionally H, C₁-C₆ alkyl, or R¹³ and R¹⁴ are joined to form a 5-6 membered alkyl containing boronate ring system;
   or a prodrug; or the compound of Formula 1 is labeled with a detectable label or an affinity tag.

In another aspect of the present invention, there is provided an intermediate compound represented by Formula 2:

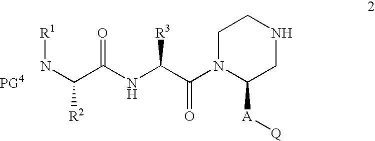

wherein PG⁴, R¹, R², R³, A and Q are as defined herein.

In another aspect of the present invention, there is provided an intermediate compound represented by Formula 3:

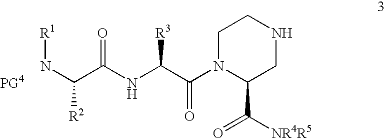

wherein PG⁴, R¹, R², R³, R⁴ and R⁵ are as defined herein.

In another aspect of the present invention, there is provided an intermediate compound represented by Formula 4:

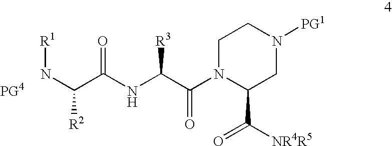

wherein PG¹, PG⁴, R¹, R², R³, R⁴ and R⁵ are as defined herein.

In another aspect of the present invention, there is provided an intermediate compound represented by Formula 5:

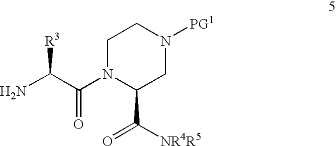

wherein PG¹, R³, R⁴ and R⁵ are as defined herein.

In another aspect of the present invention, there is provided an intermediate compound represented by Formula 6:

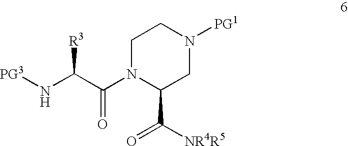

wherein PG¹, PG³, R³, R⁴ and R⁵ are as defined herein.

In another aspect of the present invention, there is provided an intermediate compound represented by Formula 7:

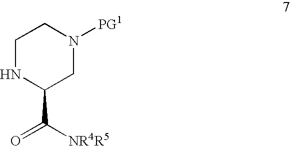

wherein PG¹, R⁴ and R⁵ are as defined herein.

In another aspect of the present invention, there is provided an intermediate compound represented by Formula 8:

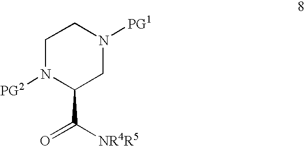

wherein PG¹, PG², R⁴ and R⁵ are as defined herein.

In another aspect of the present invention, there is provided an intermediate compound represented by Formula 9:

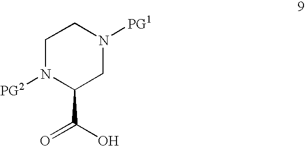

wherein PG¹ and PG² are as defined herein.

In another aspect of the present invention, there is provided an intermediate compound represented by Formula 10:

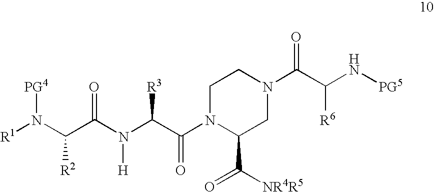

wherein PG⁴, PG⁵, R¹, R², R³, R⁴, R⁵ and R⁶ are as defined herein.

In another aspect of the present invention, there is provided an intermediate compound represented by Formula 11:

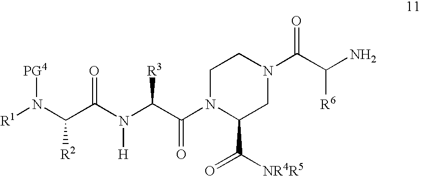

wherein PG⁴, R¹, R², R³, R⁴, R⁵ and R⁶ are as defined herein.

In another aspect of the present invention, there is provided a process for producing compounds represented by Formula 1, described hereinabove, the process comprising:
a) coupling two intermediates represented by Formula 3:

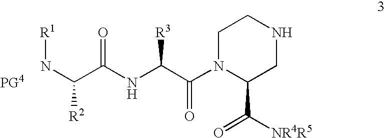

and LG-C(O)-L-C(O)-LG in a solvent with a base; and
b) removing the protecting groups so as to form compounds of Formula 12.

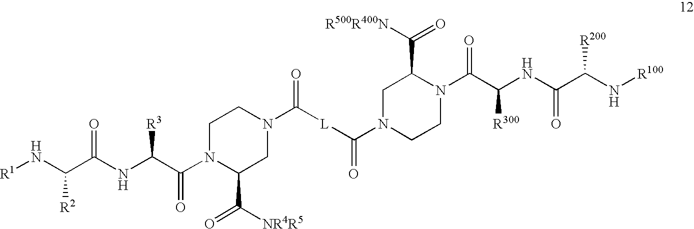

wherein L, R¹, R¹⁰⁰, R², R²⁰⁰, R³, R³⁰⁰, R⁴, R⁴⁰⁰, R⁵ and R⁵⁰⁰ are as defined herein.

In another aspect of the present invention, there is provided a process for producing compounds represented by Formula 1, described hereinabove, the process comprising:
a) coupling two intermediates represented by Formula 3:

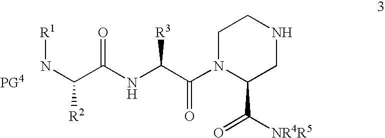

and LG-S(O)₂-L-S(O)₂-LG in a solvent with a base; and
b) removing the protecting groups so as to form compounds of Formula 13.

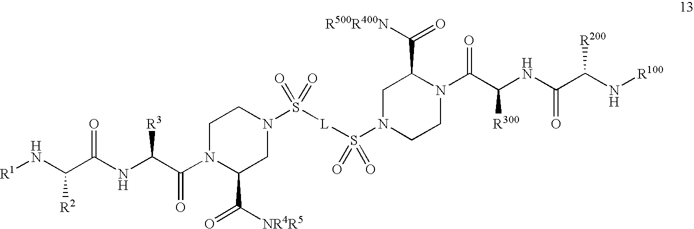

wherein L, R¹, R¹⁰⁰, R², R²⁰⁰, R³, R³⁰⁰, R⁴, R⁴⁰⁰, R⁵ and R⁵⁰⁰ are as defined herein.

In another aspect of the present invention, there is provided a process for producing compounds represented by Formula 1, described hereinabove, the process comprising:
a) coupling two intermediates represented by Formula 11:

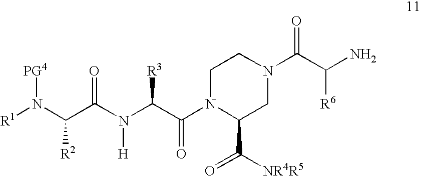

and LG(O)C-L-C(O)LG in a solvent with a base; and
b) removing the protecting groups so as to form compounds of Formula 14.

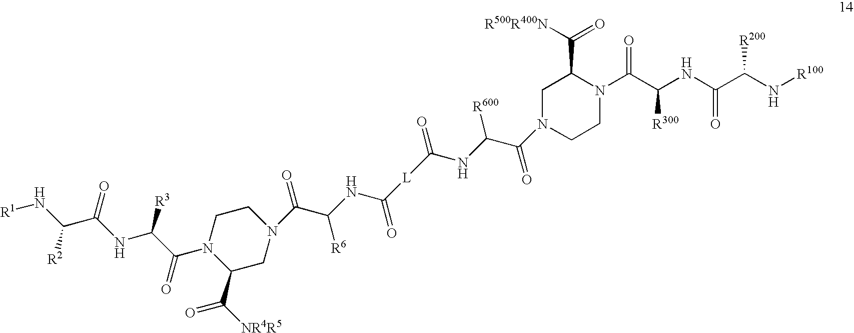

wherein L, R¹, R¹⁰⁰, R², R²⁰⁰, R³, R³⁰⁰, R⁴, R⁴⁰⁰, R⁵, R⁵⁰⁰ are as defined herein.

In another aspect of the present invention, there is provided a process for producing compounds represented by Formula 1, described hereinabove, the process comprising:
a) coupling two intermediates represented by Formula 11:

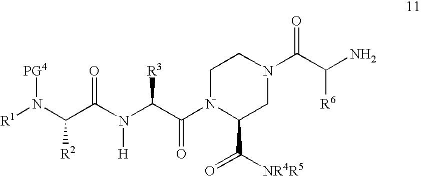

and LG(O)₂S-L-S(O)₂LG in a solvent with a base; and
b) removing the protecting groups so as to form compounds of Formula 15.

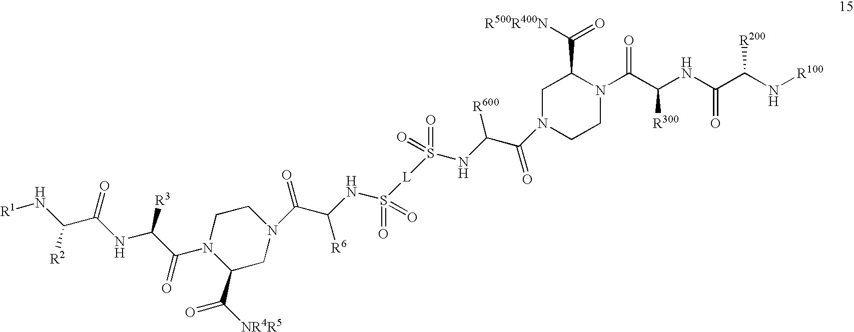

wherein L, R¹, R¹⁰⁰, R², R²⁰⁰, R³, R³⁰⁰, R⁴, R⁴⁰⁰, R⁵, R⁵⁰⁰, R⁶ and R⁶⁰⁰ are as defined herein.

In another aspect of the present invention, there is provided a method for the preparation of a pharmaceutically acceptable salt of compound of Formula 1, by the treatment of a compound of Formula 1 with 1 to 2 equivalents of a pharmaceutically acceptable acid, as defined herein, so as to form a pharmaceutically acceptable salt.

In another aspect of the present invention, there is provided a pharmaceutical composition comprising a compound, as described above, mixed with a pharmaceutically acceptable carrier, diluent or excipient.

In another aspect of the present invention, there is provided a pharmaceutical composition adapted for administration as an agent for treating a proliferative disorder in a subject, comprising a therapeutically effective amount of a compound, as described above.

In another aspect of the present invention, there is provided a pharmaceutical composition comprising a compound of Formula 1 in combination with one or more death receptor agonists, for example, a TRAIL receptor monoclonal antibody.

In another aspect of the present invention, there is provided a pharmaceutical composition comprising a compound of Formula 1 in combination with any therapeutic agent that increases the response of one or more death receptor agonists, for example cytotoxic cytokines such as interferons.

In another aspect of the present invention, there is provided a method of preparing a pharmaceutical composition, the method comprising: mixing a compound, as described above, with a pharmaceutically acceptable carrier, diluent or excipient.

In another aspect of the present invention, there is provided a method of treating a disease state characterized by insufficient apoptosis, the method comprising: administering to a subject in need thereof, a therapeutically effective amount of a compound as described above, so as to treat the disease state.

In another aspect of the present invention, there is provided a method of modulating IAP function, the method comprising: contacting a cell with a compound of the present invention so as to prevent binding of a BIR binding protein to an IAP BIR domain thereby modulating the IAP function.

In another aspect of the present invention, there is provided a method of treating a proliferative disease, the method comprising: administering to a subject in need thereof, a therapeutically effective amount of a compound as described above, so as to treat the proliferative disease.

In another aspect of the present invention, there is provided a method of treating cancer, the method comprising: administering to a subject in need thereof, a therapeutically effective amount of a compound as described above, so as to treat the cancer.

In another aspect of the present invention, there is provide a method of detecting loss of function or suppression of IAPs, the method comprising: administering to a subject, a therapeutically effective amount of a compound as described above, isolation of a tissue sample from that subject, and detection a loss of function or suppression of IAPs from that sample.

In another aspect of the present invention, there is provided a method of treating cancer, the method comprising: administering to the subject in need thereof, a therapeutically effective amount of a compound as described above, in combination or sequentially with an agent selected from:

a) an estrogen receptor modulator,
b) an androgen receptor modulator,
c) retinoid receptor modulator,
d) a cytotoxic agent,
e) an antiproliferative agent,
f) a prenyl-protein transferase inhibitor,
g) an HMG-CoA reductase inhibitor,
h) an HIV protease inhibitor,
i) a reverse transcriptase inhibitor,
k) an angiogenesis inhibitor,
l) a PPAR-.γ agonist,
m) a PPAR-.δ. agonist,
n) an inhibitor of inherent multidrug resistance,
o) an anti-emetic agent,
p) an agent useful in the treatment of anemia,
q) agents useful in the treatment of neutropenia,
r) an immunologic-enhancing drug.
s) a proteasome inhibitor;
t) an HDAC inhibitor;
u) an inhibitor of the chemotrypsin-like activity in the proteasome; or
v) E3 ligase inhibitors;
w) a modulator of the immune system such as, but not limited to, interferon-alpha, Bacillus Calmette-Guerin (BCG), and ionizing radiation (UVB) that can induce the release of cytokines, such as the interleukins, TNF, or induce release of death receptor ligands such as TRAIL;
x) a modulator of TRAIL death receptors and TRAIL receptor agonists such as the humanized antibodies HGS-ETR1 and HGS-ETR2;
or in combination or sequentially with radiation therapy, so as to treat the cancer.

In another aspect of the present invention, there is provided a method for the treatment or prevention of a proliferative disorder in a subject, the method comprising: administering to the subject a therapeutically effective amount of a compound as described above.

In another aspect of the present invention, the method further comprises administering to the subject a therapeutically effective amount of a chemotherapeutic agent prior to, simultaneously with or after administration of the compound.

In yet another aspect, the method further comprises administering to the subject a therapeutically effective amount of a death receptor agonist prior to, simultaneously with or after administration of the compound. The death receptor agonist is TRAIL or the death receptor agonist is a TRAIL receptor antibody. The death receptor agonist is typically administered in an amount that produces a synergistic effect.

In another aspect of the present invention, there is provided a probe, the probe being a compound of Formula 1 above, the compound being labeled with a detectable label or an affinity tag.

In another aspect of the present invention, there is provided a method of identifying compounds that bind to an IAP BIR domain, the assay comprising:
a) contacting an IAP BIR domain with a probe to form a probe:BIR domain complex, the probe being displaceable by a test compound;
b) measuring a signal from the probe so as to establish a reference level;
c) incubating the probe:BIR domain complex with the test compound;
d) measuring the signal from the probe;
e) comparing the signal from step d) with the reference level, a modulation of the
signal being an indication that the test compound binds to the BIR domain,
   wherein the probe is a compound of Formula 1 labeled with a detectable label or an affinity label.

In another aspect of the present invention, there is provided a use of the compound described above in the manufacture of a medicament for the treatment or prevention of a proliferative disease.

In another aspect of the present invention, there is provided a use of the compound described above in combination with a death receptor agonist in the manufacture of a medicament for the treatment of a proliferative disease.

In another aspect of the present invention, there is provided a use of the compound described above in the manufacture of a medicament for treating or preventing a disease state characterized by insufficient apoptosis.

In another aspect of the present invention, there is provided a use of the compound described above in the manufacture of a medicament for modulating IAP function.

In another aspect of the present invention, there is provided a use of the compound described above in the manufacture of a medicament for treating cancer.

In another aspect of the present invention, there is provided a use of the compound described above in combination with an agent in the manufacture of a medicament for treating cancer, where the agent is selected from:

a) an estrogen receptor modulator,
b) an androgen receptor modulator,
c) retinoid receptor modulator,
d) a cytotoxic agent,
e) an antiproliferative agent,
f) a prenyl-protein transferase inhibitor,
g) an HMG-CoA reductase inhibitor,
h) an HIV protease inhibitor,
i) a reverse transcriptase inhibitor,
k) an angiogenesis inhibitor,
l) a PPAR-.γ agonist,
m) a PPAR-.δ. agonist,
n) an inhibitor of inherent multidrug resistance,
o) an anti-emetic agent,
p) an agent useful in the treatment of anemia,
q) agents useful in the treatment of neutropenia,
r) an immunologic-enhancing drug.
s) a proteasome inhibitor;
t) an HDAC inhibitor;
u) an inhibitor of the chemotrypsin-like activity in the proteasome; or
v) E3 ligase inhibitors;
w) a modulator of the immune system such as, but not limited to, interferon-alpha, Bacillus Calmette-Guerin (BCG), and ionizing radiation (UVB) that can induce the release of cytokines, such as the interleukins, TNF, or induce release of death receptor ligands such as TRAIL;
x) a modulator of TRAIL death receptors and TRAIL receptor agonists such as the humanized antibodies HGS-ETR1 and HGS-ETR2.

In another aspect of the present invention, there is provided a use of the compound described above in the manufacture of a medicament for detecting loss or suppression of IAPs.

In another aspect of the present invention, there is provided a use of the compound described above in combination with a chemotherapeutic agent in the manufacture of a medicament for treating cancer.

In another aspect of the present invention, there is provided a use of the compound described above in combination with a death receptor agonist in the manufacture of a medicament for treating cancer.

### DETAILED DESCRIPTION OF THE INVENTION

In many cancer and other diseases, an up-regulation of IAPs induced by gene defects or by chemotherapeutic agents has been correlated to an increased resistance to apoptosis. Conversely, our results show that cells decreased in IAP levels are more sensitive to chemotherapeutic agents and to death receptor agonists such as TRAIL. It is believed that a small molecule, which will antagonize IAP function, or a loss of IAPs from diseased cells, will be useful as a therapeutic agent.

We have discovered a novel series of bridged compounds which demonstrate potent pro-apoptotic activity against human SKOV-3 ovarian cancer cell lines and HCT116 colorectal cancer cells.

The ‘bridging’ of two IAP BIR binding units, M1 and M2, described in more detail below, using an appropriate ‘bridging unit’, linked to one of the piperazine rings, provides bridged IAP BIR binding compounds, which demonstrate significantly increased anti-cancer activity (up to 10 to 1000 fold or greater than 1000 fold), as compared to their monomeric units. This improved activity results from an improved ability to bind to the BIR domains of the intact IAPs, and results in the induction of apoptosis in various cancer cell lines.

The compounds of the present invention may also be represented by Formula 1 in which M1 and M2 represent independent BIR binding domains. The compounds of formula 1 may be symmetric or asymmetric about the dotted line.

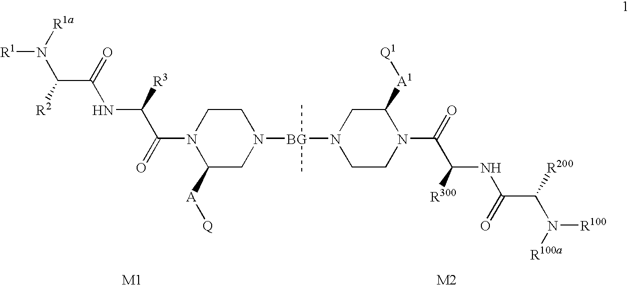

wherein R¹, R¹⁰⁰, R^{1a}, R¹⁰⁰, R², R²⁰⁰, R³, R³⁰⁰, A, A¹, Q, Q¹, and BG as defined herein, and the dotted line represents a hypothetical dividing line for comparing the substituents associated with M1 and M2.

One skilled in the art will recognize that when M1 and M2 are the same, the R¹, R^{1a}, R², R³, R⁴, R⁵, R⁶, R⁷, R⁸, R⁹, R¹⁰, R¹¹, R¹², R¹³, R¹⁴, n, m, A, Q, L¹, Y¹ and Z substituents in M1 have the same meaning as the R¹⁰⁰, R^{100a}, R²⁰⁰, R³⁰⁰, R⁴⁰⁰, R⁵⁰⁰, R⁶⁰⁰, R⁷⁰⁰, R⁸⁰⁰, R⁹⁰⁰, R¹⁰⁰⁰, R¹¹⁰⁰, R¹²⁰⁰, R¹³⁰⁰, R¹⁴⁰⁰, n, m, A¹, Q¹, L¹⁰⁰, Y¹⁰⁰, and Z substituents respectively in M2. When M1 and M2 are different, at least one at least one of the aforesaid substituents is different.

Alternatively the substituents in M1 can be defined as R¹, R^{1a}, R², R³, R⁴, R⁵, R⁶, R⁷, R⁸, R⁹, R¹⁰, R¹¹, R¹², R¹³, R¹⁴, n, m, Z, L¹, Y¹, A and Q, and those in M2 can be defined as R¹⁰⁰, R^{100a}, R²⁰⁰, R³⁰⁰, R⁴⁰⁰, R⁵⁰⁰, R⁶⁰⁰, R⁷⁰⁰, R⁸⁰⁰, R⁹⁰⁰, R¹⁰⁰⁰, R¹¹⁰⁰, R¹²⁰⁰, R¹³⁰⁰, R¹⁴⁰⁰, n, m Z, L¹⁰⁰, Y¹⁰⁰, A¹ and Q¹ respectively. In the case where M1 and M2 are the same, the R¹, R^{1a}, R², R³, R⁴, R⁵, R⁶, R⁷, R⁸, R⁹, R¹⁰, R¹¹, R¹², R¹³, R¹⁴, n, m, Z, L¹, Y¹, A and Q substituents in M1 have the same meanings as R¹⁰⁰, R^{100a}, R²⁰⁰, R³⁰⁰, R⁴⁰⁰, R⁵⁰⁰, R⁶⁰⁰, R⁷⁰⁰, R⁸⁰⁰, R⁹⁰⁰, R¹⁰⁰⁰, R¹¹⁰⁰, R¹²⁰⁰, R¹³⁰⁰, R¹⁴⁰⁰, n, m, Z, L¹⁰⁰, Y¹⁰⁰, A¹ and Q¹ respectively in M2. In the case where M1 and M2 are different, at least one of the aforesaid substituents is different.

The compounds of the present invention are useful as BIR domain binding compounds in mammalian IAPs and are represented by either Formula 1. The following are embodiments, groups and substituents of the compounds according to Formula 1, which are described hereinafter in detail.

### A and A¹:

In one subset of compounds of Formula 1, A and A¹ are both CH₂.

In an alternative subset of compounds of Formula 1, A and A¹ both are C═O.

In another alternative subset of compounds of Formula 1, A is CH₂ and A¹ is C═O.

Any and each individual definition of A and A¹ as set out herein may be combined with any and each individual definition of Core, R¹, R^{1a}, R², R¹⁰⁰, R^{100a}, R²⁰⁰, R³, R³⁰⁰, Q, Q¹, A, A¹ and BG as set out herein.

Therefore, for compounds of Formula 1, the present invention comprises compounds of Formula 1A through 1C:

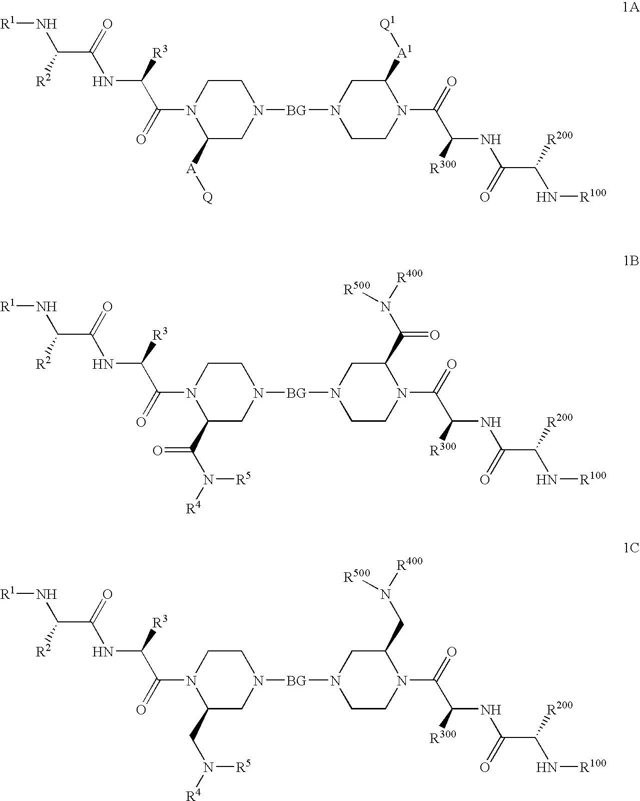

wherein BG, R¹, R^{1a}, R¹⁰⁰, R^{100a}, R², R²⁰⁰, R³, R³⁰⁰, R⁴, R⁴⁰⁰, R⁵ and R⁵⁰⁰ are as defined hereinabove and hereinafter.

In one example, the present invention comprises compounds of Formula 1A.

In an alternative example, the present invention comprises compounds of Formula 1B.

In another alternative example, the present invention comprises compounds of Formula 1C.

Any and each individual definition of Core as set out herein may be combined with any and each individual definition of A, A¹, Q, Q¹, R¹, R^{1a}, R², R³, R¹⁰⁰, R^{100a}, R²⁰⁰, R³⁰⁰ and BG as set out herein.

### BG:

In one subset of the aforesaid compounds, BG is X-L-X¹.

In another subset of the aforesaid compounds, BG is -L-.

In another subset of the aforesaid compounds, BG is

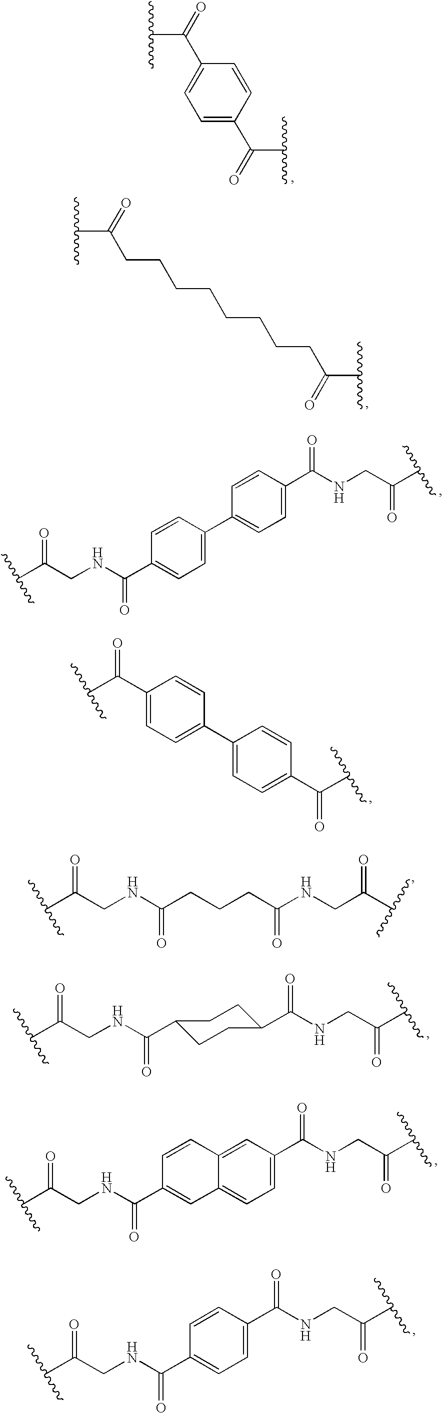

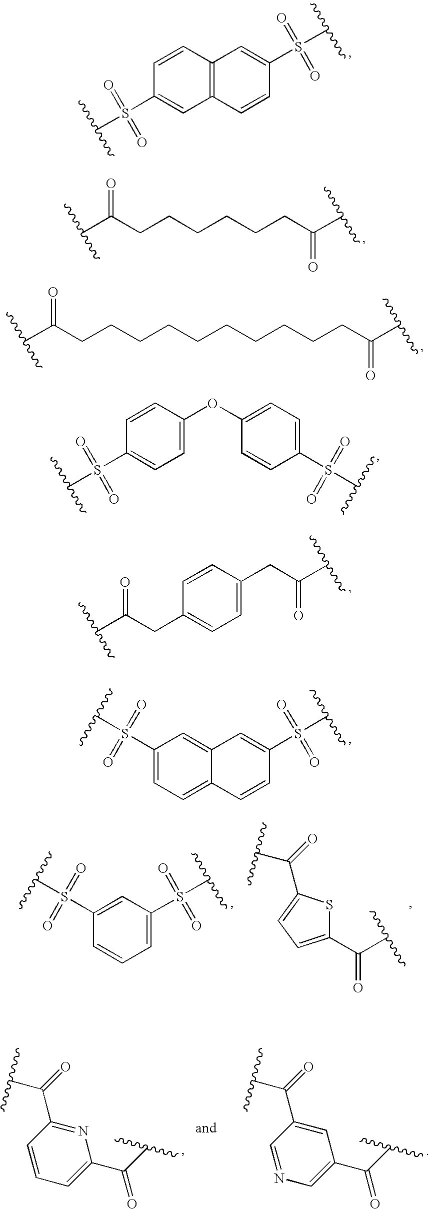

Any and each individual definition of BG as set out herein may be combined with any and each individual definition of Core, R¹, R^{1a}, R², R³, R¹⁰⁰, R^{100a}, R²⁰⁰, R³⁰⁰, A, A¹, Q, and Q¹ as set out herein.

### L

In one subset, L is selected from:
1) —C₁-C₁₂ alkyl-,
2) —C₂-C₁₂ alkenyl-,
3) —C₂-C₁₂ alkynyl-,
4) —C₃-C₇ cycloalkyl-,
5) —C₃-C₇ cycloalkenyl-,
6) 5) -aryl-,
7) -biphenyl-,
8) -heteroaryl-,
9) -heterocyclyl-,
10) —C₁-C₆ alkyl-(C₂-C₆ alkenyl)-C₁-C₆ alkyl-,
11) —C₁-C₆ alkyl-(C₂-C₄ alkynyl)-C₁-C₆ alkyl-,
12) —C₁-C₆ alkyl-(C₃-C₇ cycloalkyl)-C₁-C₆ alkyl-,
13) —C₁-C₆ alkyl-aryl-C₁-C₆ alkyl-,
14) —C₁-C₆ alkyl-biphenyl-C₁-C₆ alkyl-,
15) —C₁-C₆ alkyl-heteroaryl-C₁-C₆ alkyl-,
16) —C₁-C₆ alkyl heterocycyl-C₁-C₆ alkyl-,
17) —C₁-C₆ alkyl-Y—C₁-C₆ alkyl-, or
18) —C₁-C₆ alkyl-Z—C₁-C₆ alkyl-,
   wherein the alkyl, the alkenyl, the alkynyl, the cycloalkyenyl and the cycloalkyl are optionally substituted with one or more R⁶ substituents; and the aryl, the heteroaryl, the biphenyl and the heterocyclyl are optionally substituted with one or more R¹⁰ substituents;

Any and each individual definition of L as set out herein may be combined with any and each individual definition of Core, A, A¹, Q, Q¹, R¹, R^{1a}, R², R³, R¹⁰⁰, R^{100a}, R²⁰⁰ or R³⁰⁰, as set out herein.

### Z:

In one subset, Z is selected from:
1) —C(O)—,
2) —S(O)₂—,
3) —N(R⁸)C(O)—,
4) —C(O)N(R⁸)—,
5) —C(O)N(R⁸)—,
6) —S(O)₂N(R⁸)—,
7) —N(R⁸)—C(O)—N(R⁸)—,
8) —N(R⁸)—C(O)C(O)—N(R⁸)—,
9) —N(R⁸)—C(O)—C₁-C₁₂-alkyl-C(O)—N(R⁸)—,
10) —N(R⁸)—C(O)-aryl-C(O)—N(R⁸)—,
11) —N(R⁸)—C(O)-aryl-O-aryl-C(O)—N(R⁸)—,
12) —N(R⁸)—C(O)-heteroaryl-C(O)—N(R⁸)—,
13) —N(R⁸)—C(O)-biphenyl-C(O)—N(R⁸)—,
14) —N(R⁸)—S(O)₂—C₁-C₁₂-alkyl-S(O)₂—N(R⁸)—,
15) —N(R⁸)—S(O)₂-aryl-S(O)₂—N(R⁸)—,
16) —N(R⁸)—S(O)₂-heteroaryl-S(O)₂—N(R⁸)—,
17) —N(R⁸)—S(O)₂-biphenyl-S(O)₂—N(R⁸)—,
18) —N(R⁸)—C₁-C₁₂-alkyl-N(R⁸)—,
19) —N(R⁸)-aryl-N(R⁸)—,
20) —N(R⁸)-heteroaryl-N(R⁸)—, or
21) —N(R⁸)-biphenyl-N(R⁸)—;
   wherein the alkyl is optionally substituted with one or more R⁶ substituents, and the aryl, the heteroaryl, the biphenyl and the heterocyclyl are optionally substituted with one or more R¹⁰ substituents;

Any and each individual definition of Z as set out herein may be combined with any and each individual definition of L, R¹, R^{1a}, R², R³, R⁴, R⁵, R⁶, R⁷, R⁸, R⁹, R¹⁰, R¹¹, R¹², R¹³, R¹⁴, n, m, A, Y¹, Q, R¹⁰⁰, R^{100a}, R²⁰⁰, R³⁰⁰, R⁴⁰⁰, R⁵⁰⁰, R⁶⁰⁰, R⁷⁰⁰, R⁸⁰⁰, R⁹⁰⁰, R¹⁰⁰⁰, R¹¹⁰⁰, R¹²⁰⁰, R¹³⁰⁰, R¹⁴⁰⁰, n, m, A¹, Y¹⁰⁰ and Q¹ as set out herein.

### R¹ and R¹⁰⁰:

In one subset of the aforesaid compounds R¹ and R¹⁰⁰ are both C₁-C₆ alkyl.

In one example, R¹ and R¹⁰⁰ are both CH₃.

Any and each individual definition of R¹ and R¹⁰⁰ as set out herein may be combined with any and each individual definition of Core, A, A¹, Q¹, R^{1a}, R², R³, R^{100a}, R²⁰⁰, R³⁰⁰, and BG as set out herein.

### R^{1a} and R^{100a}:

In one subset of the aforesaid compounds R^{1a} and R^{100a} are both H.

### R² and R²⁰⁰:

In one subset of the aforesaid compounds R² and R²⁰⁰ are both C₁-C₆ alkyl.

Any and each individual definition of R² and R²⁰⁰ as set out herein may be combined with any and each individual definition of Core, A, A¹, Q, Q¹, R¹, R^{1a}, R³, R¹⁰⁰, R^{100a}, R³⁰⁰ and BG as set out herein.

### R³ and R³⁰⁰:

In one subset of compounds of Formula 1, R³ and R³⁰⁰ are both C₁-C₆ alkyl.

In one example, R³ and R³⁰⁰ are both —C(CH₃)₃.

Any and each individual definition of R³ and R³⁰⁰ as set out herein may be combined with any and each individual definition of Core, A, A¹, Q, Q¹, R¹, R^{1a}, R², R¹⁰⁰, R^{100a}, R²⁰⁰ and BG as set out herein.

### Q and Q¹:

In one subset of the aforesaid compounds, Q and Q¹ are both NR⁴R⁵, wherein R⁴ and R⁵ are as defined herein.

Any and each individual definition of Q and Q¹ as set out herein may be combined with any and each individual definition of Core, A, A¹, R¹, R^{1a}, R², R³, R¹⁰⁰, R^{100a}, R²⁰⁰, R³⁰⁰ and BG as set out herein.

### R⁴ and R⁵:

In one subset of the aforesaid compounds in which A and A¹ are both C═O, R⁴ is H and

R⁵ is selected from
1) C₁-C₆ alkyl
2) C₂-C₆ alkenyl,
3) C₂-C₄ alkynyl,
4) C₃-C₇ cycloalkyl,
5) C₃-C₇ cycloalkenyl,
6) aryl,
7) biphenyl,
8) heteroaryl,
9) heterocyclyl, or
10) heterobicyclyl,
   wherein the alkyl, alkenyl, alkynyl, cycloalkyl and cycloalkenyl are optionally substituted with one or more R⁶ substituents; and wherein the aryl, biphenyl, heteroaryl, heterocyclyl and heterobicyclyl is optionally substituted with one or more R¹⁰ substituents;
   wherein R⁶ and R¹⁰ are as defined herein.

Examples of the aforesaid subset include, R⁴ is H and R⁵ is selected from the group consisting of:

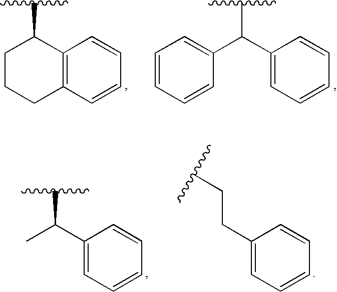

In an alternative subset of the aforesaid compounds in which A and A¹ are both CH₂, then R⁴ and R⁵ are each independently
1) haloalkyl,
2) C₁-C₆ alkyl,
3) C₂-C₆ alkenyl,
4) C₂-C₄ alkynyl,
5) C₃-C₇ cycloalkyl,
6) C₃-C₇ cycloalkenyl,
7) aryl,
8) biphenyl,
9) heteroaryl,
10) heterocyclyl,
11) heterobicyclyl,
12) —C(O)OₙR¹¹,
13) —C(═Y)NR⁸R⁹, or
14) —S(O)₂R¹¹,
   wherein the alkyl, alkenyl, alkynyl, cycloalkyl and cycloalkenyl are optionally substituted with one or more R⁶ substituents; and wherein the aryl, biphenyl, heteroaryl, heterocyclyl and heterobicyclyl is optionally substituted with one or more R¹⁰ substituents;
   wherein Y, R⁶, R⁸, R⁹, R¹⁰ and R¹¹ are as defined herein.

In another subset of the above compounds, R⁴ and R⁵ are independently selected from
1) C₁-C₆ alkyl,
2) —C(O)OₙR¹¹, or
3) —S(O)₂R¹¹,
   wherein the alkyl is substituted with an R⁶ substituent;
   wherein R⁶ and R¹¹ are as defined herein.

Any and each individual definition of R⁴ and R⁵ as set out herein may be combined with any and each individual definition of Core, A, A¹, R¹, R¹⁰⁰, R^{1a}, R^{100a}, R², R²⁰⁰, R³, R³⁰⁰ and BG as set out herein.

### R¹¹:

In one subset of the aforesaid compounds,

### R¹¹ is

1) C₁-C₆ alkyl, or
2) aryl,
   wherein the alkyl is optionally substituted with one or more R⁶ substituents; and wherein the aryl is optionally substituted with one or more R¹⁰ substituents;
   wherein R⁶ and R¹⁰ are as defined herein.

In one subset of the aforesaid compounds, R¹¹ is
1) C₁-C₆ alkyl optionally substituted with one or two R⁶ substituents, or
2) phenyl optionally substituted with one R¹⁰ substituent;
   wherein the R⁶ and the R¹⁰ substituents are as defined herein.

Any and each individual definition of R¹¹ as set out herein may be combined with any and each individual definition of Core, A, A¹, R¹, R^{1a}, R², R³, R⁴, R⁵, R¹⁰⁰, R^{100a}, R²⁰⁰, R³⁰⁰, R⁴⁰⁰, R⁵⁰⁰ and BG as set out herein.

### R⁶:

In one subset of the aforesaid compounds, R⁶ is
1) halogen,
2) —NO₂,
3) —CN,
4) aryl,
5) biphenyl,
6) heteroaryl,
7) heterocyclyl,
8) heterobicyclyl,
9) —OR⁷,
10) —SR⁷, or
11) —NR⁸R⁹,
   wherein the aryl, biphenyl, heteroaryl, heterocyclyl and heterobicyclyl is optionally substituted with one or more R¹⁰ substituents;
   wherein R⁷, R⁸, R⁹ and R¹⁰ are as defined herein.

In another subset of the aforesaid compounds, R⁶ is
1) halogen,
2) aryl, or
3) —NR⁸R⁹,
   wherein the aryl is optionally substituted with one R¹⁰ substituent;
   wherein R⁸, R⁹ and R¹⁰ are as defined herein.

In one subset of the aforesaid compounds, R⁶ is
1) halogen,
2) phenyl, or
3) —NR⁸R⁹,
   wherein the phenyl is optionally substituted with one R¹⁰ substituent;
   wherein R⁸, R⁹ and R¹⁰ are as defined herein.

Any and each individual definition of R⁶ as set out herein may be combined with any and each individual definition of Core, A, A¹, R¹, R^{1a}, R², R³, R⁴, R⁵, R¹⁰⁰, R^{100a}, R²⁰⁰, R³⁰⁰, R⁴⁰⁰, R⁵⁰⁰ and BG as set out herein.

### R⁷:

1) H,
2) haloalkyl,
3) C₁-C₆ alkyl,
4) C₂-C₆ alkenyl,
5) C₂-C₄ alkynyl,
6) C₃-C₇ cycloalkyl,
7) C₃-C₇ cycloalkenyl,
8) aryl,
9) biphenyl,
10) heteroaryl,
11) heterocyclyl,
12) heterobicyclyl,
13) —C₁-C₆ alkyl-C₂-C₄ alkenyl, or
14) —C₁-C₆ alkyl-C₂-C₄ alkynyl,
   wherein the alkyl, alkenyl, alkynyl, cycloalkyl and cycloalkenyl are optionally substituted with one or more R⁶ substituents; and wherein the aryl, biphenyl, heteroaryl, heterocyclyl and heterobicyclyl are optionally substituted with one or more R¹⁰ substituents;

### R⁸ and R⁹:

In one subset of the aforesaid compounds, R⁸ and R⁹ are each independently
1) H,
2) haloalkyl,
3) C₁-C₆ alkyl,
4) C₂-C₆ alkenyl,
5) C₂-C₄ alkynyl,
6) C₃-C₇ cycloalkyl, or
7) C₃-C₇ cycloalkenyl,
   wherein the alkyl, alkenyl, alkynyl, cycloalkyl and cycloalkenyl are optionally substituted with one or more R⁶ substituents;
   wherein the R⁶ substituents are as defined herein.

In another subset of the aforesaid compounds, R⁸ and R⁹ are each independently
1) H, or
2) C₁-C₆ alkyl,
   wherein the alkyl is optionally substituted with an aryl.

Any and each individual definition of R⁸ and R⁹ as set out herein may be combined with any and each individual definition of Core, A, A¹, R¹, R^{1a}, R², R³, R⁴, R⁵, R¹⁰⁰, R^{100a}, R²⁰⁰, R³⁰⁰, R⁴⁰⁰, R⁵⁰⁰ and BG as set out herein.

### R¹⁰:

In one aspect of the aforesaid compounds, R¹⁰ is
1) halogen,
2) NO₂,
3) CN,
4) haloalkyl,
5) —OR⁷,
6) —NR⁸R⁹, or
7) —SR⁷;
   wherein R⁷, R⁸, and R⁹ are as defined herein.

In another aspect of the aforesaid compounds, R¹⁰ is
1) halogen, or
2) —OC₁-C₆ alkyl.

Any and each individual definition of R¹⁰ as set out herein may be combined with any and each individual definition of Core, A, A¹, R¹, R^{1a}, R², R³, R⁴, R⁵, R¹⁰⁰, R^{100a}, R²⁰⁰, R³⁰⁰, R⁴⁰⁰, R⁵⁰⁰ and BG as set out herein.

In one embodiment of the present invention, there is provided an isomer, enantiomer, diastereoisomer or tautomer of a compound represented by Formula 1:

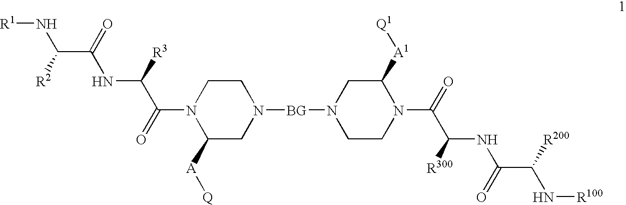

or a salt thereof,
wherein
n is 0 or 1;
m is 0, 1 or 2;

### Y is NR⁸, O or S;

### BG is

3) —X-L-X¹—; or
4) L;
   X and X¹ are independently selected from

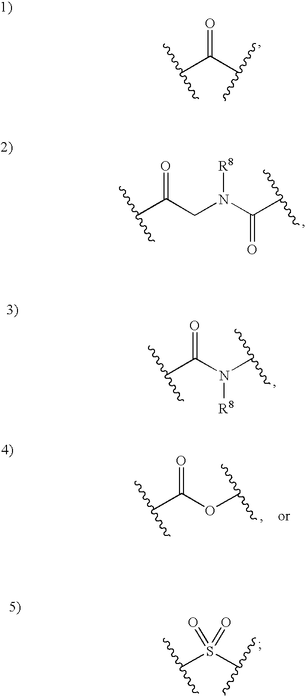

L is selected from:
1) —C₁-C₂₀ alkyl-,
2) —C₂-C₆ alkenyl-,
3) —C₂-C₄ alkynyl-,
4) —C₃-C₇ cycloalkyl-,
5) -aryl-,
6) -biphenyl-,
7) -heteroaryl-,
8) -heterocyclyl-,
9) —C₁-C₆ alkyl-(C₂-C₆ alkenyl)-C₁-C₆ alkyl-,
10) —C₁-C₆ alkyl-(C₂-C₄ alkynyl)-C₁-C₆ alkyl-
11) —C₁-C₆ alkyl-(C₃-C₇ cycloalkyl)-C₁-C₆ alkyl-,
12) —C₁-C₆ alkyl-aryl-C₁-C₆ alkyl-,
13) —C₁-C₆ alkyl-biphenyl-C₁-C₆ alkyl-,
14) —C₁-C₆ alkyl-heteroaryl-C₁-C₆ alkyl-,
15) —C₁-C₆ alkyl-heterocycyl-C₁-C₆ alkyl-,
16) —C₁-C₆ alkyl-Y—C₁-C₆ alkyl-,
17) -aryl-Y-aryl-,
18) —C₁-C₆ alkyl-Z—C₁-C₆ alkyl, or
19) -aryl-Z-aryl-.
wherein the alkyl, alkenyl, alkynyl and cycloalkyl are optionally substituted with one or more R⁶ substituents, and the aryl, biphenyl, heteroaryl, and heterocyclyl are optionally substituted with one or more R¹⁰ substituents;
   Z is selected from:
22) —C(O)—,
23) —S(O)₂—,
24) —N(R⁸)C(O)—,
25) —C(O)N(R⁸)—,
26) —C(O)N(R⁸)—,
27) —S(O)₂N(R⁸)—,
28) —N(R⁸)—C(O)—N(R⁸)—,
29) —N(R⁸)—C(O)C(O)—N(R⁸)—,
30) —N(R⁸)—C(O)—C₁-C₁₂-alkyl-C(O)—N(R⁸)—,
31) —N(R⁸)—C(O)-aryl-C(O)—N(R⁸)—,
32) —N(R⁸)—C(O)-aryl-O-aryl-C(O)—N(R⁸)—,
33) —N(R⁸)—C(O)-heteroaryl-C(O)—N(R⁸)—,
34) —N(R⁸)—C(O)-biphenyl-C(O)—N(R⁸)—,
35) —N(R⁸)—S(O)₂—C₁-C₁₂-alkyl-S(O)₂—N(R⁸)—,
36) —N(R⁸)—S(O)₂-aryl-S(O)₂—N(R⁸)—,
37) —N(R⁸)—S(O)₂-heteroaryl-S(O)₂—N(R⁸)—,
38) —N(R⁸)—S(O)₂-biphenyl-S(O)₂—N(R⁸)—,
39) —N(R⁸)—C₁-C₁₂-alkyl-N(R⁸)—,
40) —N(R⁸)-aryl-N(R⁸)—,
41) —N(R⁸)-heteroaryl-N(R⁸)—, or
42) —N(R⁸)-biphenyl-N(R⁸)—;
   wherein the alkyl is optionally substituted with one or more R⁶ substituents, and the aryl, the heteroaryl, the biphenyl and the heterocyclyl are optionally substituted with one or more R¹⁰ substituents;
   Q and Q¹ are independently selected from
4) —NR⁴R⁵,
5) —OR¹¹,
6) —S(O)ₘR¹¹; or
   Q and Q¹ are independently selected from
3) aryl, or
4) heteroaryl.
   wherein the aryl and the heteroaryl are optionally substituted with one or more R¹⁰ substituents;
   A and A¹ are independently selected from
8) —CH₂—,
9) —CH₂CH₂—,
10) —CH(C₁-C₆ alkyl)-,
11) —CH(C₃-C₇ cycloalkyl)-,
12) —C₃-C₇ cycloalkyl-,
13) —CH(C₁-C₆ alkyl-C₃-C₇ cycloalkyl)-,
14) —C(O)—, or
   wherein the alkyl and cycloalkyl are optionally substituted with one or more R⁶ substituents;
   R¹, R^{1a}, R¹⁰⁰ and R^{100a} are independently selected from
3) H, or
4) C₁-C₆ alkyl optionally substituted with one or more R⁶ substituents;
   R² and R²⁰⁰ are independently selected from
3) H, or
4) C₁-C₆ alkyl optionally substituted with one or more R⁶ substituents;
   R³ and R³⁰⁰ are independently selected from
10) H,
11) C₁-C₆ alkyl,
12) C₃-C₇ cycloalkyl,
13) C₃-C₇ cycloalkenyl,
14) aryl,
15) biphenyl,
16) heteroaryl,
17) heterocyclyl,
18) heterobicyclyl,
   wherein the alkyl, cycloalkyl and cycloalkenyl are optionally substituted with one or more R⁶ substituents; and wherein the aryl, biphenyl, heteroaryl, heterocyclyl and heterobicyclyl are optionally substituted with one or more R¹⁰ substituents.
   R⁴ and R⁵ are each independently selected from
19) H,
20) haloalkyl,
21) C₁-C₆ alkyl,
22) C₂-C₆ alkenyl,
23) C₂-C₄ alkynyl,
24) C₃-C₇ cycloalkyl,
25) C₃-C₇ cycloalkenyl,
26) aryl,
27) biphenyl,
28) heteroaryl,
29) heterocyclyl,
30) heterobicyclyl,
31) aryl-heteroaryl,
32) heteroaryl-aryl,
33) heterocyclyl-aryl,
34) —C(O)Oₙ—R¹¹,
35) —S(O)₂—R¹¹, or
36) —C(═Y)NR⁸R⁹,
   wherein the alkyl, the alkenyl, the alkynyl, the cycloalkyl and the cycloalkenyl are optionally substituted with one or more R⁶ substituents, and the aryl, the biphenyl, the heteroaryl, the heterocyclyl and the heterobicyclyl are optionally substituted with one or more R¹⁰ substituents

### R⁶ is

25) halogen,
26) NO₂,
27) CN,
28) haloalkyl,
29) C₁-C₆ alkyl,
30) C₂-C₆ alkenyl,
31) C₂-C₄ alkynyl,
32) C₃-C₇ cycloalkyl,
33) C₃-C₇ cycloalkenyl,
34) aryl,
35) heteroaryl,
36) heterocyclyl,
37) heterobicyclyl,
38) —OR⁷,
39) —S(O)ₘR⁷,
40) —NR⁸R⁹,
41) —NR⁸S(O)₂R¹¹,
42) —C(O)OₙR⁷,
43) —C(O)NR⁸R⁹,
44) —S(O)₂NR⁸R⁹
45) —OC(O)R⁷,
46) —OC(O)Y—R¹¹,
47) —SC(O)R⁷, or
48) —NC(Y)NR⁸R⁹.
   wherein the aryl, heteroaryl, heterocyclyl and heterobicyclyl is optionally substituted with one or more R¹⁰ substituents

### R⁷ is

15) H,
16) haloalkyl,
17) C₁-C₆ alkyl,
18) C₂-C₆ alkenyl,
19) C₂-C₄ alkynyl,
20) C₃-C₇ cycloalkyl,
21) C₃-C₇ cycloalkenyl,
22) aryl,
23) biphenyl,
24) heteroaryl,
25) heterocyclyl,
26) heterobicyclyl,
27) —C₁-C₆ alkyl-C₂-C₄ alkenyl, or
28) —C₁-C₆ alkyl-C₂-C₄ alkynyl,
   wherein the alkyl, alkenyl, alkynyl, cycloalkyl and cycloalkenyl are optionally substituted with one or more R⁶ substituents; and wherein the aryl, biphenyl, heteroaryl, heterocyclyl and heterobicyclyl are optionally substituted with one or more R¹⁰ substituents;
   R⁸ and R⁹ are each independently
13) H,
14) -haloalkyl,
15) —C₁-C₆ alkyl,
16) —C₂-C₆ alkenyl,
17) —C₂-C₄ alkynyl,
18) —C₃-C₇ cycloalkyl,
19) —C₃-C₇ cycloalkenyl,
20) aryl,
21) biphenyl,
22) heteroaryl,
23) heterocyclyl,
24) heterobicyclyl,
   wherein the alkyl, alkenyl, alkynyl, cycloalkyl and cycloalkenyl are optionally substituted with one or more R⁶ substituents; and wherein the aryl, biphenyl, heteroaryl, heterocyclyl and heterobicyclyl are optionally substituted with one or more R¹⁰ substituents;
   or R⁸ and R⁹ together with the nitrogen atom to which they are bonded form a five, six or seven membered heterocyclic ring optionally substituted with one or more R⁶ substituents;

### R¹⁰ is

24) halogen,
25) NO₂,
26) CN,
27) —B(OR¹³)(OR¹⁴),
28) —C₁-C₆ alkyl,
29) —C₂-C₆ alkenyl,
30) —C₂-C₄ alkynyl,
31) —C₃-C₇ cycloalkyl,
32) —C₃-C₇ cycloalkenyl,
33) haloalkyl,
34) —OR⁷,
35) —NR⁸R⁹,
36) —SR⁷,
37) —COR⁷,
38) —C(O)OR⁷,
39) —S(O)ₘR⁷,
40) —CONR⁸R⁹,
41) —S(O)₂NR⁸R⁹,
42) aryl,
43) biphenyl,
44) heteroaryl,
45) heterocyclyl, or
46) heterobicyclyl,
   wherein the alkyl, alkenyl, alkynyl, cycloalkyl and cycloalkenyl are optionally substituted with one or more R⁶ substituents;

### R¹¹ is

12) haloalkyl,
13) C₁-C₆ alkyl,
14) C₂-C₆ alkenyl,
15) C₂-C₄ alkynyl,
16) C₃-C₇ cycloalkyl,
17) C₃-C₇ cycloalkenyl,
18) aryl,
19) biphenyl,
20) heteroaryl,
21) heterocyclyl, or
22) heterobicyclyl,
   wherein the alkyl, alkenyl, alkynyl, cycloalkyl and cycloalkenyl are optionally substituted with one or more R⁶ substituents; and wherein the aryl, biphenyl, heteroaryl, heterocyclyl and heterobicyclyl are optionally substituted with one or more R¹⁰ substituents;

### R¹² is

17) haloalkyl,
18) C₁-C₆ alkyl,
19) C₂-C₆ alkenyl,
20) C₂-C₄ alkynyl,
21) C₃-C₇ cycloalkyl,
22) C₃-C₇ cycloalkenyl,
23) aryl,
24) biphenyl,
25) heteroaryl,
26) heterocyclyl,
27) heterobicyclyl,
28) —C(O)—R¹¹,
29) —C(O)O—R¹¹,
30) —C(O)NR⁸R⁹,
31) —S(O)ₘ—R¹¹, or
32) —C(═Y)NR⁸R⁹,
   wherein the alkyl, alkenyl, alkynyl, cycloalkyl and cycloalkenyl are optionally substituted with one or more R⁶ substituents; and wherein the aryl, biphenyl, heteroaryl, heterocyclyl and heterobicyclyl are optionally substituted with one or more R¹⁰ substituents;
   R¹³ and R¹⁴ are optionally H, C₁-C₆ alkyl, or R¹³ and R¹⁴ are joined to form a 5-6 membered alkyl containing boronate ring system;
   or a prodrug; or the compound of Formula 1 is labeled with a detectable label or an affinity tag.

If any variable, such as R⁶, R⁶⁰⁰, R¹⁰, R¹⁰⁰⁰ and the like, occurs more than one time in any constituent structure, the definition of the variable at each occurrence is independent at every other occurrence. If a substituent is itself substituted with one or more substituents, it is to be understood that that the one or more substituents may be attached to the same carbon atom or different carbon atoms. Combinations of substituents and variables defined herein are allowed only if they produce chemically stable compounds.

One skilled in the art will understand that substitution patterns and substituents on compounds of the present invention may be selected to provide compounds that are chemically stable and can be readily synthesized using the chemistry set forth in the examples and chemistry techniques well known in the art using readily available starting materials.

It is to be understood that many substituents or groups described herein have functional group equivalents, which means that the group or substituent may be replaced by another group or substituent that has similar electronic, hybridization or bonding properties.

### DEFINITIONS

Unless otherwise specified, the following definitions apply:

The singular forms “a”, “an” and “the” include corresponding plural references unless the context clearly dictates otherwise.

As used herein, the term “comprising” is intended to mean that the list of elements following the word “comprising” are required or mandatory but that other elements are optional and may or may not be present.

As used herein, the term “consisting of” is intended to mean including and limited to whatever follows the phrase “consisting of”. Thus the phrase “consisting of” indicates that the listed elements are required or mandatory and that no other elements may be present.

As used herein, the term “alkyl” is intended to include both branched and straight chain saturated aliphatic hydrocarbon groups having the specified number of carbon atoms, for example, C₁-C₆ as in C₁-C₆-alkyl is defined as including groups having 1, 2, 3, 4, 5 or 6 carbons in a linear or branched arrangement, and C₁-C₄ as in C₁-C₄ alkyl is defined as including groups having 1, 2, 3, or 4 carbons in a linear or branched arrangement, and for example, C₁-C₂₀ as in C₁-C₂₀-alkyl is defined as including groups having 1, 2, 3, 4, 5, 6, 7, 8, 9, 10, 11, 12, 13, 14, 15, 16, 17, 18, 19 or 20 carbons in a linear or branched arrangement, Examples of C₁-C₆-alkyl and C₁-C₄ alkyl as defined above include, but are not limited to, methyl, ethyl, n-propyl, i-propyl, n-butyl, t-butyl, i-butyl, pentyl and hexyl.

As used herein, the term, “alkenyl” is intended to mean unsaturated straight or branched chain hydrocarbon groups having the specified number of carbon atoms therein, and in which at least two of the carbon atoms are bonded to each other by a double bond, and having either E or Z regeochemistry and combinations thereof. For example, C₂-C₆ as in C₂-C₆ alkenyl is defined as including groups having 2, 3, 4, 5, or 6 carbons in a linear or branched arrangement, at least two of the carbon atoms being bonded together by a double bond. Examples of C₂-C₆ alkenyl include ethenyl (vinyl), 1-propenyl, 2-propenyl, 1-butenyl and the like.

As used herein, the term “alkynyl” is intended to mean unsaturated, straight chain hydrocarbon groups having the specified number of carbon atoms therein and in which at least two carbon atoms are bonded together by a triple bond. For example C₂-C₄ as in C₂-C₄ alkynyl is defined as including groups having 2, 3, or 4 carbon atoms in a chain, at least two of the carbon atoms being bonded together by a triple bond. Examples of such alkynyls include ethynyl, 1-propynyl, 2-propynyl and the like.

As used herein, the term “cycloalkyl” is intended to mean a monocyclic saturated aliphatic hydrocarbon group having the specified number of carbon atoms therein, for example, C₃-C₇ as in C₃-C₇ cycloalkyl is defined as including groups having 3, 4, 5, 6, or 7 carbons in a monocyclic arrangement. Examples of C₃-C₇ cycloalkyl as defined above include, but are not limited to, cyclopropyl, cyclobutyl, cyclopentyl, cyclohexyl and cycloheptyl.

As used herein, the term “cycloalkenyl” is intended to mean a monocyclic saturated aliphatic hydrocarbon group having the specified number of carbon atoms therein, for example, C₃-C₇ as in C₃-C₇ cycloalkenyl is defined as including groups having 3, 4, 5, 6, or 7 carbons in a monocyclic arrangement. Examples of C₃-C₇ cycloalkenyl as defined above include, but are not limited to, cyclopentenyl, and cyclohexenyl.

As used herein, the term “halo” or “halogen” is intended to mean fluorine, chlorine, bromine and iodine.

As used herein, the term “haloalkyl” is intended to mean an alkyl as defined above, in which each hydrogen atom may be successively replaced by a halogen atom. Examples of haloalkyls include, but are not limited to, CH₂F, CHF₂ and CF₃.

As used herein, the term “aryl”, either alone or in combination with another radical, means a carbocyclic aromatic monocyclic group containing 6 carbon atoms which may be further fused to a second 5- or 6-membered carbocyclic group which may be aromatic, saturated or unsaturated. Aryl includes, but is not limited to, phenyl, indanyl, 1-naphthyl, 2-naphthyl and tetrahydronaphthyl. The aryls may be connected to another group either at a suitable position on the cycloalkyl ring or the aromatic ring. For example:

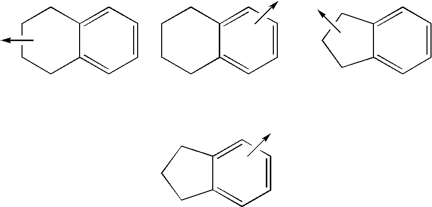

Arrowed lines drawn from the ring system indicate that the bond may be attached to any of the suitable ring atoms.

As used herein, the term “biphenyl” is intended to mean two phenyl groups bonded together at any one of the available sites on the phenyl ring. For example:

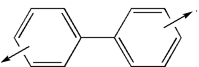

As used herein, the term “heteroaryl” is intended to mean a monocyclic or bicyclic ring system of up to ten atoms, wherein at least one ring is aromatic, and contains from 1 to 4 hetero atoms selected from the group consisting of O, N, and S. The heteroaryl substituent may be attached either via a ring carbon atom or one of the heteroatoms. Examples of heteroaryl groups include, but are not limited to thienyl, benzimidazolyl, benzobthienyl, furyl, benzofuranyl, pyranyl, isobenzofuranyl, chromenyl, xanthenyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, napthyridinyl, quinoxalinyl, quinazolinyl, cinnolinyl, pteridinyl, isothiazolyl, isochromanyl, chromanyl, isoxazolyl, furazanyl, indolinyl, isoindolinyl, thiazolo4,5-b-pyridine, and

fluoroscein derivatives such as:

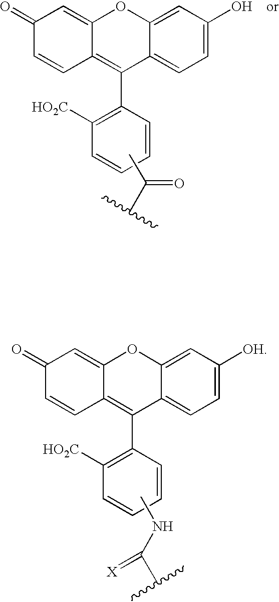

As used herein, the term “heterocycle”, “heterocyclic” or “heterocyclyl” is intended to mean a 5, 6, or 7 membered non-aromatic ring system containing from 1 to 4 heteroatoms selected from the group consisting of O, N and S. Examples of heterocycles include, but are not limited to pyrrolidinyl, tetrahydrofuranyl, piperidyl, pyrrolinyl, piperazinyl, imidazolidinyl, morpholinyl, imidazolinyl, pyrazolidinyl, pyrazolinyl, and biotinyl,

As used herein, the term “heterobicycle” either alone or in combination with another radical, is intended to mean a heterocycle as defined above fused to another cycle, be it a heterocycle, an aryl or any other cycle defined herein. Examples of such heterobicycles include, but are not limited to, coumarin, benzod1,3dioxole, 2,3-dihydrobenzob1,4dioxine and 3,4-dihydro-2H-benzob1,4dioxepine.

As used herein, the term “heteroatom” is intended to mean O, S or N.

As used herein, the term “activated diacid” is intended to mean a diacid wherein the carboxylic acid moieties have been transformed to, for example, but not limited to, acid halides, a succinate esters, or HOBt esters, either in situ or in a separate synthetic step. For example, succinyl chloride and terephthaloyl chloride are examples of “diacid chlorides”. HOBt esters can be formed in situ by the treatment of a diacid with a dehydrating agent such as DCC, EDC, HBTU, or others, a base such as DIPEA, and HOBt in an appropriate solvent. The reaction of an activated diacid with an amine will result in the conversion of the acid functionality to an amide functionality.

As used herein, the term “detectable label” is intended to mean a group that may be linked to a compound of the present invention to produce a probe or to an IAP BIR domain, such that when the probe is associated with the BIR domain, the label allows either direct or indirect recognition of the probe so that it may be detected, measured and quantified. As used herein, the term “affinity tag” is intended to mean a ligand or group, which is linked to either a compound of the present invention or to an IAP BIR domain to allow another compound to be extracted from a solution to which the ligand or group is attached.

As used herein, the term “probe” is intended to mean a compound of Formula 1 which is labeled with either a detectable label or an affinity tag, and which is capable of binding, either covalently or non-covalently, to an IAP BIR domain. When, for example, the probe is non-covalently bound, it may be displaced by a test compound. When, for example, the probe is bound covalently, it may be used to form cross-linked adducts, which may be quantified and inhibited by a test compound.

As used herein, the term “optionally substituted with one or more substituents” or its equivalent term “optionally substituted with at least one substituent” is intended to mean that the subsequently described event of circumstances may or may not occur, and that the description includes instances where the event or circumstance occurs and instances in which it does not. The definition is intended to mean from zero to five substituents.

If the substituents themselves are incompatible with the synthetic methods of the present invention, the substituent may be protected with a suitable protecting group (PG) that is stable to the reaction conditions used in these methods. The protecting group may be removed at a suitable point in the reaction sequence of the method to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3^{rd} ed.), John Wiley & Sons, NY (1999), which is incorporated herein by reference in its entirety. Examples of protecting groups used throughout include, but are not limited to Fmoc, Bn, Boc, CBz and COCF₃. In some instances, a substituent may be specifically selected to be reactive under the reaction conditions used in the methods of this invention. Under these circumstances, the reaction conditions convert the selected substituent into another substituent that is either useful in an intermediate compound in the methods of this invention or is a desired substituent in a target compound.

Abbreviations for α-amino acids used throughout are as follows:

| Amino acid | Abbreviation |
|---|---|
| α-Amino butyric acid | Abu |
| Alanine | Ala |
| Arginine | Arg |
| Aspartic acid | Asp |
| Asparagine | Asn |
| Cysteine | Cys |
| Glutamic acid | Glu |
| Glutamine | Gln |
| Glycine | Gly |
| Isoleucine | Ile |
| Histidine | His |
| Leucine | Leu |
| Lysine | Lys |
| Methionine | Met |
| Phenylalanine | Phe |
| Proline | Pro |
| Serine | Ser |
| Threonine | Thr |
| Tryptophan | Trp |
| Tyrosine | Tyr |
| Valine | Val |

As used herein, the term “residue” when referring to α-amino acids is intended to mean a radical derived from the corresponding α-amino acid by eliminating the hydroxyl of the carboxy group and one hydrogen of the α-amino group. For example, the terms Gln, Ala, Gly, Ile, Arg, Asp, Phe, Ser, Leu, Cys, Asn, and Tyr represent the residues of L-glutamine, L-alanine, glycine, L-isoleucine, L-arginine, L-aspartic acid, L-phenylalanine, L-serine, L-leucine, L-cysteine, L-asparagine, and L-tyrosine, respectively.

As used herein, the term “subject” is intended to mean humans and non-human mammals such as primates, cats, dogs, swine, cattle, sheep, goats, horses, rabbits, rats, mice and the like.

As used herein, the term “prodrug” is intended to mean a compound that may be converted under physiological conditions or by solvolysis to a biologically active compound of the present invention. Thus, the term “prodrug” refers to a precursor of a compound of the invention that is pharmaceutically acceptable. A prodrug may be inactive or display limited activity when administered to a subject in need thereof, but is converted in vivo to an active compound of the present invention. Typically, prodrugs are transformed in vivo to yield the compound of the invention, for example, by hydrolysis in blood or other organs by enzymatic processing. The prodrug compound often offers advantages of solubility, tissue compatibility or delayed release in the subject (see, Bundgard, H., Design of Prodrugs (1985), pp. 7-9, 21-24 (Elsevier, Amsterdam). The definition of prodrug includes any covalently bonded carriers which release the active compound of the invention in vivo when such prodrug is administered to a subject. Prodrugs of a compound of the present invention may be prepared by modifying functional groups present in the compound of the invention in such a way that the modifications are cleaved, either in routine manipulation or in vivo, to a parent compound of the invention.

As used herein, the term “pharmaceutically acceptable carrier, diluent or excipient” is intended to mean, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, emulsifier, or encapsulating agent, such as a liposome, cyclodextrins, encapsulating polymeric delivery systems or polyethyleneglycol matrix, which is acceptable for use in the subject, preferably humans.

As used herein, the term “pharmaceutically acceptable salt” is intended to mean both acid and base addition salts.

As used herein, the term “pharmaceutically acceptable acid addition salt” is intended to mean those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

As used herein, the term “pharmaceutically acceptable base addition salt” is intended to mean those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like.

As used herein, the term “BIR domain binding” is intended to mean the action of a compound of the present invention upon an IAP BIR domain, which blocks or diminishes the binding of IAPs to BIR binding proteins or is involved in displacing BIR binding proteins from an IAP. Examples of BIR binding proteins include, but are not limited to, caspases and mitochondrially derived BIR binding proteins such as Smac, Omi/WTR2A and the like.

As used herein, the term “insufficient apoptosis” is intended to mean a state wherein a disease is caused or continues because cells deleterious to the subject have not apoptosed. This includes, but is not limited to, cancer cells that survive in a subject without treatment, cancer cells that survive in a subject during or following anti-cancer treatment, or immune cells whose action is deleterious to the subject, and includes, neutrophils, monocytes and auto-reactive T-cells.

As used herein, the term “therapeutically effective amount” is intended to mean an amount of a compound of Formula 1 which, when administered to a subject is sufficient to effect treatment for a disease-state associated with insufficient apoptosis. The amount of the compound of Formula 1 will vary depending on the compound, the condition and its severity, and the age of the subject to be treated, but can be determined routinely by one of ordinary skill in the art having regard to his own knowledge and to this disclosure.

As used herein, the term “treating” or “treatment” is intended to mean treatment of a disease-state associated with insufficient apoptosis, as disclosed herein, in a subject, and includes: (i) preventing a disease or condition associated with insufficient apoptosis from occurring in a subject, in particular, when such mammal is predisposed to the disease or condition but has not yet been diagnosed as having it; (ii) inhibiting a disease or condition associated with insufficient apoptosis, i.e., arresting its development; or (iii) relieving a disease or condition associated with insufficient apoptosis, i.e., causing regression of the condition.

As used herein, the term “treating cancer” is intended to mean the administration of a pharmaceutical composition of the present invention to a subject, preferably a human, which is afflicted with cancer to cause an alleviation of the cancer by killing, inhibiting the growth, or inhibiting the metastasis of the cancer cells.

As used herein, the term “preventing disease” is intended to mean, in the case of cancer, the post-surgical, post-chemotherapy or post-radiotherapy administration of a pharmaceutical composition of the present invention to a subject, preferably a human, which was afflicted with cancer to prevent the regrowth of the cancer by killing, inhibiting the growth, or inhibiting the metastasis of any remaining cancer cells. Also included in this definition is the prevention of prosurvival conditions that lead to diseases such as asthma, rheumatoid arthritis, MS and the like.

As used herein, the term “synergistic effect” is intended to mean that the effect achieved with the combination of the compounds of the present invention and either the chemotherapeutic agents or death receptor agonists of the invention is greater than the effect which is obtained with only one of the compounds, agents or agonists, or advantageously the effect which is obtained with the combination of the above compounds, agents or agonists is greater than the addition of the effects obtained with each of the compounds, agents or agonists used separately. Such synergy enables smaller doses to be given.

As used herein, the term “apoptosis” or “programmed cell death” is intended to mean the regulated process of cell death wherein a dying cell displays a set of well-characterized biochemical hallmarks that include cell membrane blebbing, cell soma shrinkage, chromatin condensation, and DNA laddering, as well as any caspase-mediated cell death.

As used herein, the term “BIR domain” or “BIR” are used interchangeably throughout and are intended to mean a domain which is characterized by a number of invariant amino acid residue including conserved cysteines and one conserved histidine residue within the sequence Cys-(Xaa1)₂Cys-(Xaa1)₁₆His-(Xaa1)₆₋₈Cys. Typically, the amino acid sequence of the consensus sequence is: Xaa1-Xaa1-Xaa1-Arg-Leu-Xaa1-Thr-Phe-Xaa1-Xaa1-Trp-Pro-Xaa2-Xaa1-Xaa1-Xaa2-Xaa2-Xaa1-Xaa1-Xaa1-Xaa1-Leu-Ala-Xaa1-Ala-Gly-Phe-Tyr-Tyr-Xaa1-Gly-Xaa1-Xaa1-Asp-Xaa1-Val-Xaa1-Cys-Phe-Xaa1-Cys-Xaa1-Xaa1-Xaa1-Xaa1-Xaa1-Xaa1-Trp-Xaa1-Xaa1-Xaa1-Asp-Xaa1-Xaa1-Xaa1-Xaa1-Xaa1-His-Xaa-1-Xaa1-Xaa1-Xaa1-Pro-Xaa1-Cys-Xaa1-Phe-Val, wherein Xaa1 is any amino acid and Xaa2 is any amino acid or is absent. Preferably the sequence is substantially identical to one of the BIR domain sequences provided for XIAP, HIAP1, or HIAP2 herein. The BIR domain residues are listed below (see Genome Biology (2001) 1-10):

| | XIAP | HIAP-1 | HIAP-2 |
|---|---|---|---|
| BIR1 | 21-93 | 41-113 | 24-96 |
| BIR2 | 159-230 | 179-250 | 164-235 |
| BIR3 | 258-330 | 264-336 | 250-322 |
| Seq. # | P98170 | XP-006266 | XP-006267 |

As used herein, the term “ring zinc finger” or “RZF” is intended to mean a domain having the amino acid sequence of the consensus sequence: Glu-Xaa1-Xaa1-Xaa1-Xaa1-Xaa1-Xaa-1-Xaa2-Xaa1-Xaa1-Xaa1-Cys-Lys-Xaa3-Cys-Met-Xaa1-Xaa1-Xaa1-Xaa1-Xaa1-Xaa3-X-aa1-Phe-Xaa1-Pro-Cys-Gly-His-Xaa1-Xaa1-Xaa1-Cys-Xaa1-Xaa1-Cys-Ala-Xaa1-Xaa-1-Xaa1-Xaa1-Xaa1-Cys-Pro-Xaa1-Cys, wherein Xaa1 is any amino acid, Xaa2 is Glu or Asp, and Xaa3 is Val or Ile.

As used herein, the term “IAP” is intended to mean a polypeptide or protein, or fragment thereof, encoded by an IAP gene. Examples of IAPs include, but are not limited to human or mouse NAIP (Birc 1), HIAP-1 (cIAP2, Birc 3), HIAP-2 (cIAP1, Birc 2), XIAP (Birc 4), survivin (Birc 5), livin (ML-IAP, Birc 7), ILP-2 (Birc 8) and Apollon/BRUCE (Birc 6) (see for example U.S. Pat. Nos. 6,107,041; 6,133,437; 6,156,535; 6,541,457; 6,656,704; 6,689,562; Deveraux and Reed, Genes Dev. 13, 239-252, 1999; Kasof and Gomes, J. Biol. Chem., 276, 3238-3246, 2001; Vucic et al., Curr. Biol. 10, 1359-1366, 2000; Ashab et al. FEBS Lett., 495, 56-60, 2001, the contents of which are hereby incorporated by reference).

As used herein, the term “IAP gene” is intended to mean a gene encoding a polypeptide having at least one BIR domain and which is capable of modulating (inhibiting or enhancing) apoptosis in a cell or tissue. The IAP gene is a gene having about 50% or greater nucleotide sequence identity to at least one of human or mouse NAIP (Birc 1), HIAP-1 (cIAP2, Birc 3), HIAP-2 (cIAP1, Birc 2), XIAP (Birc 4), survivin (Birc 5), livin (ML-IAP, Birc 7), ILP-2 (Birc 8) and Apollon/BRUCE (Birc 6). The region of sequence over which identity is measured is a region encoding at least one BIR domain and a ring zinc finger domain. Mammalian IAP genes include nucleotide sequences isolated from any mammalian source.

As used herein, the term “IC₅₀” is intended to mean an amount, concentration or dosage of a particular compound of the present invention that achieves a 50% inhibition of a maximal response, such as displacement of maximal fluorescent probe binding in an assay that measures such response.

As used herein, the term “EC₅₀” is intended to mean an amount, concentration or dosage of a particular compound of the present invention that achieves a 50% inhibition of cell survival.

As used herein, the term “modulate” or “modulating” is intended to mean the treatment, prevention, suppression, enhancement or induction of a function or condition using the compounds of the present invention. For example, the compounds of the present invention can modulate IAP function in a subject, thereby enhancing apoptosis by significantly reducing, or essentially eliminating the interaction of activated apoptotic proteins, such as caspase-3, 7 and 9, with the BIR domains of mammalian IAPs or by inducing the loss of XIAP protein in a cell.

As used herein, the term “enhancing apoptosis” is intended to mean increasing the number of cells that apoptose in a given cell population either in vitro or in vivo. Examples of cell populations include, but are not limited to, ovarian cancer cells, colon cancer cells, breast cancer cells, lung cancer cells, pancreatic cancer cells, or T cells and the like. It will be appreciated that the degree of apoptosis enhancement provided by an apoptosis-enhancing compound of the present invention in a given assay will vary, but that one skilled in the art can determine the statistically significant change in the level of apoptosis that identifies a compound that enhances apoptosis otherwise limited by an IAP. Preferably “enhancing apoptosis” means that the increase in the number of cells undergoing apoptosis is at least 25%, more preferably the increase is 50%, and most preferably the increase is at least one-fold. Preferably the sample monitored is a sample of cells that normally undergo insufficient apoptosis (i.e., cancer cells). Methods for detecting the changes in the level of apoptosis (i.e., enhancement or reduction) are described in the Examples and include methods that quantitate the fragmentation of DNA, methods that quantitate the translocation phosphatoylserine from the cytoplasmic to the extracellular side of the membrane, determination of activation of the caspases and methods quantitate the release of cytochrome C and the apoptosis inhibitory factor into the cytoplasm by mitochondria.

As used herein, the term “proliferative disease” or “proliferative disorder” is intended to mean a disease that is caused by or results in inappropriately high levels of cell division, inappropriately low levels of apoptosis, or both. For example, cancers such as lymphoma, leukemia, melanoma, ovarian cancer, breast cancer, pancreatic cancer, and lung cancer, and autoimmune disorders are all examples of proliferative diseases.

As used herein, the term “death receptor agonist” is intended to mean an agent capable of stimulating by direct or indirect contact the pro apoptotic response mediated by the death-receptors. For example, an agonist TRAIL receptor antibody would bind to TRAIL receptor (S) and trigger an apoptotic response. On the other hand, other agents such as interferon-alpha could trigger the release of endogeneous TRAIL and/or up regulate the TRAIL receptors in such a way that the cell pro-apoptotic response of the cell is amplified.

The compounds of the present invention, or their pharmaceutically acceptable salts may contain one or more asymmetric centers, chiral axes and chiral planes and may thus give rise to enantiomers, diastereomers, and other stereoisomeric forms and may be defined in terms of absolute stereochemistry, such as (R)- or (S)- or, as (D)- or (L)- for amino acids. The present invention is intended to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. The racemic mixtures may be prepared and thereafter separated into individual optical isomers or these optical isomers may be prepared by chiral synthesis. The enantiomers may be resolved by methods known to those skilled in the art, for example by formation of diastereoisomeric salts which may then be separated by crystallization, gas-liquid or liquid chromatography, selective reaction of one enantiomer with an enantiomer specific reagent. It will also be appreciated by those skilled in the art that where the desired enantiomer is converted into another chemical entity by a separation technique, an additional step is then required to form the desired enantiomeric form. Alternatively specific enantiomers may be synthesized by asymmetric synthesis using optically active reagents, substrates, catalysts, or solvents or by converting one enantiomer to another by asymmetric transformation.

Certain compounds of the present invention may exist in Zwitterionic form and the present invention includes Zwitterionic forms of these compounds and mixtures thereof.

### Utilities

The compounds of the present invention are useful as IAP BIR domain binding compounds and as such the compounds, compositions and method of the present invention include application to the cells or subjects afflicted with or having a predisposition towards developing a particular disease state, which is characterized by insufficient apoptosis. Thus, the compounds, compositions and methods of the present invention are used to treat cellular proliferative diseases/disorders, which include, but are not limited to, i) cancer, ii) autoimmune disease, iii) inflammatory disorders, iv) proliferation induced post medical procedures, including, but not limited to, surgery, angioplasty, and the like.

The compounds of the present invention may also be useful in the treatment of diseases in which there is a defect in the programmed cell-death or the apoptotic machinery (TRAIL, FAS, apoptosome), such as multiple sclerosis, atherosclerosis, inflammation, autoimmunity and the like.

The treatment involves administration to a subject in need thereof a compound of the present invention or a pharmaceutically acceptable salt thereof, or a pharmaceutical composition comprising a pharmaceutical carrier and a therapeutically effective amount of a compound of the present invention, or a pharmaceutically acceptable salt thereof. In particular, the compounds, compositions and methods of the present invention are useful for the treatment of cancer including solid tumors such as skin, breast, brain, lung, testicular carcinomas, and the like. Cancers that may be treated by the compounds, compositions and methods of the invention include, but are not limited to the following:

| Tissue | Example |
|---|---|
| Adrenal gland | neuroblastoma |
| Bone | osteogenic sarcoma (osteosarcoma), fibrosarcoma, malignant |
| | fibrous histiocytoma, chondrosarcoma, Ewing's sarcoma, |
| | malignant lymphoma (reticulum cell sarcoma), multiple |
| | myeloma, malignant giant cell tumor chordoma, |
| | osteochronfroma (osteocartilaginous exostoses), benign |
| | chondroma, chondroblastoma, chondromyxofibroma, osteoid |
| | osteoma and giant cell tumors |
| Cardiac | sarcoma (angiosarcoma, fibrosarcoma, rhabdomyosarcoma, |
| | liposarcoma), myxoma, rhabdomyoma, fibroma, lipoma and teratoma |
| Gastrointestinal | esophagus (squamous cell carcinoma, adenocarcinoma, |
| | leiomyosarcoma, lymphoma), stomach (carcinoma, lymphoma, |
| | leiomyosarcoma), pancreas (ductal adenocarcinoma, |
| | insulinoma, glucagonoma, gastrinoma, carcinoid tumors, |
| | vipoma), small bowel (adenocarcinoma, lymphoma, carcinoid |
| | tumors, Karposi's sarcoma, leiomyoma, hemangioma, lipoma, |
| | neurofibroma, fibroma), large bowel (adenocarcinoma, tubular |
| | adenoma, villous adenoma, hamartoma, leiomyoma) |
| Genitourinary | kidney (adenocarcinoma, Wilm's tumor nephroblastoma, |
| tract | lymphoma, leukemia), bladder and urethra (squamous cell |
| | carcinoma, transitional cell carcinoma, adenocarcinoma), |
| | prostate (adenocarcinoma, sarcoma), testis (seminoma, |
| | teratoma, embryonal carcinoma, teratocarcinoma, |
| | choriocarcinoma, sarcoma, interstitial cell carcinoma, fibroma, |
| | fibroadenoma, adenomatoid tumors, lipoma) |
| Gynecological | uterus (endometrial carcinoma), cervix (cervical carcinoma, |
| | pre-tumor cervical dysplasia), ovaries (ovarian carcinoma |
| | serous cystadenocarcinoma, mucinous cystadenocarcinoma, |
| | unclassified carcinoma, granulosa-thecal cell tumors, Sertoli- |
| | Leydig cell tumors, dysgerminoma, malignant teratoma), vulva |
| | (squamous cell carcinoma, intraepithelial carcinoma, |
| | adenocarcinoma, fibrosarcoma, melanoma), vagina (clear cell |
| | carcinoma, squamous cell carcinoma, botryoid sarcoma |
| | (embryonal rhabdomyosarcoma), fallopian tubes (carcinoma) |
| Hematologic | blood (myeloid leukemia acute and chronic, acute |
| | lymphoblastic leukemia, chronic lymphocytic leukemia, |
| | myeloproliferative diseases, multiple myeloma, myelodysplastic |
| | syndrome), Hodgkin's disease, non-Hodgkin's lymphoma |
| | malignant lymphoma |
| Liver | hepatoma (hepatocellular carcinoma), cholangiocarcinoma, |
| | hepatoblastoma, angiosarcoma, hepatocellular adenoma, |
| | hemangioma |
| Lung | bronchogenic carcinoma (squamous cell, undifferentiated small |
| | cell, undifferentiated large cell, adenocarcinoma), alveolar |
| | (bronchiolar) carcinoma, bronchial adenoma, sarcoma, |
| | lymphoma, chondromatous hamartoma, mesothelioma |
| Nervous system | skull (osteoma, hemangioma, granuloma, xanthoma, osteitis |
| | deformans), meninges (meningioma, meningiosarcoma, |
| | gliomatosis), brain (astrocytoma, medulloblastoma, glioma, |
| | ependymoma, germinoma pinealoma, glioblastoma multiform, |
| | oligodendroglioma, schwannoma, retinoblastoma, congenital |
| | tumors), spinal cord neurofibroma, meningioma, glioma, |
| | sarcoma) |
| Skin | malignant melanoma, basal cell carcinoma, squamous cell |
| | carcinoma, Karposi's sarcoma, moles dysplastic nevi, lipoma, |
| | angioma, dermatofibroma, keloids |

The compounds of the present invention, or their pharmaceutically acceptable salts or their prodrugs, may be administered in pure form or in an appropriate pharmaceutical composition, and can be carried out via any of the accepted modes of Galenic pharmaceutical practice.

The pharmaceutical compositions of the present invention can be prepared by mixing a compound of the present invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral (subcutaneous injections, intravenous, intramuscular, intrasternal injection or infusion techniques), sublingual, ocular, rectal, vaginal, and intranasal. Pharmaceutical compositions of the present invention are formulated so as to allow the active ingredients contained therein to be bioavailable upon administration of the composition to a subject. Compositions that will be administered to a subject or patient take the form of one or more dosage units, where for example, a tablet may be a single dosage unit, and a container of a compound of the present invention in aerosol form may hold a plurality of dosage units. Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the present invention, or a pharmaceutically acceptable salt thereof, for treatment of a disease-state as described above.

A pharmaceutical composition of the present invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example inhalatory administration.

For oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, e.g., a gelatin capsule, it may contain, in addition to materials of the above type, a liquid carrier such as polyethylene glycol or oil such as soybean or vegetable oil.

The pharmaceutical composition may be in the form of a liquid, e.g., an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the present invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; encapsulating agents such as cyclodextrins or functionalized cyclodextrins, including, but not limited to, α, β, or δ-hydroxypropylcyclodextins or Captisol; antioxidants such as ascorbic acid or sodium bisulfite; chelating agents such as ethylenediamine tetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

A liquid pharmaceutical composition of the present invention used for either parenteral or oral administration should contain an amount of a compound of the present invention such that a suitable dosage will be obtained. Typically, this amount is at least 0.01% of a compound of the present invention in the composition. When intended for oral administration, this amount may be varied to be between 0.1 and about 70% of the weight of the composition. For parenteral usage, compositions and preparations according to the present invention are prepared so that a parenteral dosage unit contains between 0.01 to 10% by weight of the compound of the present invention. Pharmaceutical compositions may be further diluted at the time of administration; for example a parenteral formulation may be further diluted with a sterile, isotonic solution for injection such as 0.9% saline, 5 wt % dextrose (D5W), Ringer's solution, or others.

The pharmaceutical composition of the present invention may be used for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of the present invention from about 0.1 to about 10% w/v (weight per unit volume).

The pharmaceutical composition of the present invention may be used for rectal administration to treat for example, colon cancer, in the form, e.g., of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the present invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the present invention in solid or liquid form may include an agent that binds to the compound of the present invention and thereby assists in the delivery of the compound. Suitable agents that may act in this capacity include, but are not limited to, a monoclonal or polyclonal antibody, a protein or a liposome.

The pharmaceutical composition of the present invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the present invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the present invention may be prepared by methodology well known in the pharmaceutical art. For example, a pharmaceutical composition intended to be administered by injection can be prepared by admixing a compound of the present invention with sterile, distilled water so as to form a solution. A surfactant may be added to facilitate the formation of a homogeneous solution or suspension. Surfactants are compounds that non-covalently interact with the compound of the present invention so as to facilitate dissolution or homogeneous suspension of the compound in the aqueous delivery system.

The compounds of the present invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy. Generally, a therapeutically effective daily dose may be from about 0.1 mg to about 40 mg/kg of body weight per day or twice per day of a compound of the present invention, or a pharmaceutically acceptable salt thereof.

### Combination Therapy

The compounds of the present invention, or pharmaceutically acceptable salts thereof, may also be administered simultaneously with, prior to, or after administration of one or more of the therapeutic agents described below. Such combination therapy may include administration of a single pharmaceutical dosage formulation which contains a compound of the present invention and one or more additional agents given below, as well as administration of the compound of the present invention and each additional agent in its own separate pharmaceutical dosage formulation. For example, a compound of the present invention and a chemotherapeutic agent, such as taxol (paclitaxel), taxotere, etoposide, cisplatin, vincristine, vinblastine, bortezomib, doxorubicin, sorafenib, and the like, can be administered to the patient either together in a single oral dosage composition such as a tablet or capsule, or each agent administered in separate oral dosage formulations or via intravenous injection. Where separate dosage formulations are used, the compounds of the present invention and one or more additional agents can be administered at essentially the same time, i.e., concurrently, or at separately staggered times, i.e., sequentially; combination therapy is understood to include all these regimens. In addition, these compounds may synergize with molecules that may stimulate the death receptor apoptotic pathway through a direct or indirect manner, as for example, the compounds of the present invention may be used in combination with soluble TRAIL or with any agent or procedure that can cause an increase in circulating level of TRAIL, such as interferon-alpha or radiation.

Thus, the present invention also encompasses the use of the compounds of the present invention in combination with radiation therapy or one or more additional agents such as those described in WO 03/099211 (PCT/US03/15861), which is hereby incorporated by reference.

Examples of such additional agents include, but are not limited to the following:

a) an estrogen receptor modulator,
b) an androgen receptor modulator,
c) retinoid receptor modulator,
d) a cytotoxic agent,
e) an antiproliferative agent,
f) a prenyl-protein transferase inhibitor,
g) an HMG-CoA reductase inhibitor,
h) an HIV protease inhibitor,
i) a reverse transcriptase inhibitor,
k) an angiogenesis inhibitor,
l) a PPAR-.γ agonist,
m) a PPAR-.δ. agonist,
n) an inhibitor of inherent multidrug resistance,
o) an anti-emetic agent,
p) an agent useful in the treatment of anemia,
q) agents useful in the treatment of neutropenia,
r) an immunologic-enhancing drug.
s) a proteasome inhibitor such as Velcade and MG132 (7-Leu-Leu-aldehyde) (see He at al. in Oncogene (2004) 23, 2554-2558);
t) an HDAC inhibitor, such as sodium butyrate, phenyl butyrate, hydroxamic acids, cyclin tetrapeptide and the like (see Rosato et al., Molecular Cancer Therapeutics 2003, 1273-1284);
u) an inhibitor of the chemotrypsin-like activity in the proteasome;
v) E3 ligase inhibitors;
w) a modulator of the immune system such as interferon-alpha and ionizing radiation (UVB) that can induce the release of cytokines, such as the interleukins, TNF, or induce release of Death receptor Ligands such as TRAIL;
x) a modulator of TRAIL death receptors and TRAIL receptor agonists such as the humanized antibodies HGS-ETR¹ and HGS-ETR²; and
or in combination or sequentially with radiation therapy, so as to treat the cancer.

Additional combinations may also include agents which reduce the toxicity of the aforesaid agents, such as hepatic toxicity, neuronal toxicity, nephrotoxicity and the like.

In one example, co-administration of one of the compounds of Formula 1 of the present invention with a death receptor agonist such as TRAIL, such as a small molecule or an antibody that mimics TRAIL may cause an advantageous synergistic effect. Moreover, the compounds of the present invention may be used in combination with any compounds that cause an increase in circulating levels of TRAIL.

### Vinca Alkaloids and Related Compounds

Vinca alkaloids that can be used in combination with the nucleobase oligomers of the invention to treat cancer and other neoplasms include vincristine, vinblastine, vindesine, vinflunine, vinorelbine, and anhydrovinblastine.

Dolastatins are oligopeptides that primarily interfere with tubulin at the vinca alkaloid binding domain. These compounds can also be used in combination with the compounds of the invention to treat cancer and other neoplasms. Dolastatins include dolastatin-10 (NCS 376128), dolastatin-15, ILX651, TZT-1027, symplostatin 1, symplostatin 3, and LU103793 (cemadotin).

Cryptophycins (e.g., cryptophycin 1 and cryptophycin 52 (LY355703)) bind tubulin within the vinca alkaloid-binding domain and induce G2/M arrest and apoptosis. Any of these compounds can be used in combination with the compounds of the invention to treat cancer and other neoplasms.

Other microtubule disrupting compounds that can be used in conjunction with the compounds of the invention to treat cancer and other neoplasms are described in U.S. Pat. Nos. 6,458,765; 6,433,187; 6,323,315; 6,258,841; 6,143,721; 6,127,377; 6,103,698; 6,023,626; 5,985,837; 5,965,537; 5,955,423; 5,952,298; 5,939,527; 5,886,025; 5,831,002; 5,741,892; 5,665,860; 5,654,399; 5,635,483; 5,599,902; 5,530,097; 5,521,284; 5,504,191; 4,879,278; and 4,816,444, and U.S. patent application Publication Nos. 2003/0153505 A1; 2003/0083263 A1; and 2003/0055002 A1, each of which is hereby incorporated by reference.

### Taxanes and Other Microtubule Stabilizing Compounds

Taxanes such as paclitaxel, doxetaxel, RPR 109881A, SB-T-1213, SB-T-1250, SB-T-101187, BMS-275183, BRT 216, DJ-927, MAC-321, IDN5109, and IDN5390 can be used in combination with the compounds of the invention to treat cancer and other neoplasms. Taxane analogs (e.g., BMS-184476, BMS-188797) and functionally related non-taxanes (e.g., epothilones (e.g., epothilone A, epothilone B (EPO906), deoxyepothilone B, and epothilone B lactam (BMS-247550)), eleutherobin, discodermolide, 2-epi-discodermolide, 2-des-methyldiscodermolide, 5-hydroxymethyldiscoder-molide, 19-des-aminocarbonyldiscodermolide, 9(13)-cyclodiscodermolide, and laulimalide) can also be used in the methods and compositions of the invention.

Other microtubule stabilizing compounds that can be used in combination with the compounds of the invention to treat cancer and other neoplasms are described in U.S. Pat. Nos. 6,624,317; 6,610,736; 6,605,599; 6,589,968; 6,583,290; 6,576,658; 6,515,017; 6,531,497; 6,500,858; 6,498,257; 6,495,594; 6,489,314; 6,458,976; 6,441,186; 6,441,025; 6,414,015; 6,387,927; 6,380,395; 6,380,394; 6,362,217; 6,359,140; 6,306,893; 6,302,838; 6,300,355; 6,291,690; 6,291,684; 6,268,381; 6,262,107; 6,262,094; 6,147,234; 6,136,808; 6,127,406; 6,100,411; 6,096,909; 6,025,385; 6,011,056; 5,965,718; 5,955,489; 5,919,815; 5,912,263; 5,840,750; 5,821,263; 5,767,297; 5,728,725; 5,721,268; 5,719,177; 5,714,513; 5,587,489; 5,473,057; 5,407,674; 5,250,722; 5,010,099; and 4,939,168; and U.S. patent application Publication Nos. 2003/0186965 A1; 2003/0176710 A1; 2003/0176473 A1; 2003/0144523 A1; 2003/0134883 A1; 2003/0087888 A1; 2003/0060623 A1; 2003/0045711 A1; 2003/0023082 A1; 2002/0198256 A1; 2002/0193361 A1; 2002/0188014 A1; 2002/0165257 A1; 2002/0156110 A1; 2002/0128471 A1; 2002/0045609 A1; 2002/0022651 A1; 2002/0016356 A1; 2002/0002292 A1, each of which is hereby incorporated by reference.

### TRAIL Receptor Agonist Antibodies

Agonist antibodies directed against the death receptors TRAIL-R1 and/or TRAIL-R² can be used in combination with compounds of the invention. Exemplary agonist antibodies that may be used in combination with compounds of the invention include those described in U.S. Pat. No. 7,244,429; in U.S. Patent Application Publication Nos. 2007/0179086, 2002/0004227, 2006/0269554, 2005/0079172, 2007/0292411, 2006/0270837, 2006/0269555, 2004/0214235, and 2007/0298039; and in International Patent Publications WO2006/017961 and WO98/51793. Each of these publications is hereby incorporated by reference in its entirety. In preferred embodiments, compounds of the invention are used in combination with one or more of these TRAIL receptor agonist antibodies for the treatment of cancer and other neoplasms.

Other chemotherapeutic agents that may be administered with a compound of the present invention are listed in the following Table:

| | | |
|---|---|---|
| Alkylating | cyclophosphamide | mechlorethamine |
| agents | lomustine | thiotepa |
| | busulfan | streptozocin |
| | procarbazine | chlorambucil |
| | ifosfamide | temozolomide |
| | altretamine | dacarbazine |
| | melphalan | semustine |
| | estramustine phosphate | carmustine |
| | hexamethylmelamine | |
| Platinum agents | cisplatin | tetraplatin |
| | carboplatinum | BBR-3464 (Hoffmann-La Roche) |
| | oxaliplatin | Ormiplatin |
| | ZD-0473 (AnorMED) | SM-11355 (Sumitomo) |
| | spiroplatinum | iproplatin |
| | lobaplatin (Aeterna) | AP-5280 (Access) |
| | carboxyphthalatoplatinum | |
| | satraplatin (Johnson Matthey) | |
| Antimetabolites | azacytidine | 6-mercaptopurine |
| | tomudex | hydroxyurea |
| | gemcitabine | 6-thioguanine |
| | trimetrexate | decitabine (SuperGen) |
| | capecitabine | cytarabin |
| | deoxycoformycin | clofarabine (Bioenvision) |
| | 5-fluorouracil | 2-fluorodeoxy |
| | fludarabine | cytidine |
| | floxuridine | irofulven (MGI Pharma) methotrexate |
| | pentostatin | DMDC (Hoffmann-La Roche) |
| | 2-chlorodeoxyadenosine | idatrexate |
| | raltitrexed | ethynylcytidine (Taiho) |
| Topoisomerase | amsacrine | TAS-103 (Taiho) |
| inhibitors | rubitecan (SuperGen) | Topotecan |
| | epirubicin | elsamitrucin (Spectrum) dexrazoxanet |
| | exatecan mesylate (Daiichi) | (TopoTarget) |
| | etoposide | J-107088 (Merck & Co) |
| | quinamed (ChemGenex) | pixantrone (Novuspharma) |
| | teniposide or mitoxantrone | BNP-1350 (BioNumerik) |
| | gimatecan (Sigma-Tau) | rebeccamycin analogue (Exelixis) |
| | irinotecan (CPT-11) | CKD-602 (Chong Kun Dang) |
| | diflomotecan (Beaufour-Ipsen) | BBR-3576 (Novuspharma) |
| | 7-ethyl-10-hydroxy-camptothecin | KW-2170 (Kyowa Hakko) |
| Antitumor | dactinomycin (actinomycin D) | bleomycinic acid |
| antibiotics | amonafide | idarubicin |
| | doxorubicin (adriamycin) | bleomycin A |
| | azonafide | rubidazone |
| | deoxyrubicin | bleomycin B |
| | anthrapyrazole | plicamycinp |
| | valrubicin | mitomycin C |
| | oxantrazole | porfiromycin |
| | daunorubicin (daunomycin) | MEN-10755 (Menarini) |
| | losoxantrone | cyanomorpholinodoxorubicin |
| | epirubicin | GPX-100 (Gem Pharmaceuticals) |
| | bleomycin sulfate (blenoxane) | mitoxantrone (novantrone) |
| | therarubicin | |
| Antimitotic | paclitaxel | RPR 109881A (Aventis) |
| agents | SB 408075 (GlaxoSmithKline) | ZD 6126 (AstraZeneca) |
| | docetaxel | TXD 258 (Aventis) |
| | E7010 (Abbott) | PEG-paclitaxel (Enzon) |
| | Colchicines | epothilone B (Novartis) |
| | PG-TXL (Cell Therapeutics) | AZ10992 (Asahi) |
| | vinblastine | T 900607 (Tularik) |
| | IDN 5109 (Bayer) | IDN-5109 (Indena) |
| | Vincristine | T 138067 (Tularik) |
| | A 105972 (Abbott) | AVLB (Prescient NeuroPharma) |
| | Vinorelbine | cryptophycin 52 (Eli Lilly) |
| | A 204197 (Abbott) | azaepothilone B (BMS) |
| | Vindesine | vinflunine (Fabre) |
| | LU 223651 (BASF) | BNP-7787 (BioNumerik) |
| | dolastatin 10 (NCI) | auristatin PE (Teikoku Hormone) |
| | D 24851 (ASTAMedica) | CA-4 prodrug (OXiGENE) |
| | rhizoxin (Fujisawa) | BMS 247550 (BMS) |
| | ER-86526 (Eisai) | dolastatin-10 (NIH) |
| | mivobulin (Warner-Lambert) | BMS 184476(BMS) |
| | combretastatin A4 (BMS) | CA-4 (OXiGENE) |
| | cemadotin (BASF) | BMS 188797 (BMS) |
| | isohomohalichondrin-B (PharmaMar) | taxoprexin (Protarga) |
| Aromatase | Aminoglutethimide | anastrazole |
| inhibitors | Exemestane | YM-511 (Yamanouchi) |
| | Letrozole | formestane |
| | atamestane (BioMedicines) | |
| Thymidylate | pemetrexed (Eli Lilly) | ZD-9331 (BTG) |
| synthase | nolatrexed (Eximias) | CoFactor ™ (BioKeys) |
| inhibitors | | |
| DNA | trabectedin (PharmaMar) | albumin + 32P (Isotope Solutions) |
| antagonists | mafosfamide (Baxter International) | O6 benzyl guanine (Paligent) |
| | glufosfamide (Baxter International) | thymectacin (NewBiotics) edotreotide |
| | apaziquone (Spectrum | (Novartis) |
| | Pharmaceuticals) | |
| Farnesyltransferase | arglabin (NuOncology Labs) | perillyl alcohol (DOR BioPharma) |
| inhibitors | tipifarnib (Johnson & Johnson) | BAY-43-9006 (Bayer) |
| | lonafarnib (Schering-Plough) | |
| Pump inhibitors | CBT-1 (CBA Pharma) | tariquidar (Xenova) |
| | zosuquidar trihydrochloride (Eli | biricodar dicitrate (Vertex) |
| | Lilly) | MS-209 (Schering AG) |
| Histone | tacedinaline (Pfizer) | depsipeptide (Fujisawa) |
| acetyltransferase | pivaloyloxymethyl butyrate (Titan) | MS-275 (Schering AG) |
| inhibitors | SAHA (Aton Pharma) | |
| Metalloproteinase | Neovastat (Aeterna Laboratories) | marimastat (British Biotech) BMS- |
| inhibitors | CMT-3 (CollaGenex) | 275291 (Celltech) |
| Ribonucleoside | gallium maltolate (Titan) | triapine (Vion) |
| reductase | tezacitabine (Aventis) | didox (Molecules for Health) |
| inhibitors | | |
| TNF alpha | virulizin (Lorus Therapeutics) | CDC-394 (Celgene) |
| agonists/antagonists | revimid (Celgene) | |
| Endothelin A | atrasentan (Abbott) | ZD-4054 (AstraZeneca) |
| receptor | YM-598 (Yamanouchi) | |
| antagonist | | |
| Retinoic acid | fenretinide (Johnson & Johnson) | LGD-1550 (Ligand) |
| receptor agonists | alitretinoin (Ligand) | |
| Immuno- | Interferon | norelin (Biostar) |
| modulators | dexosome therapy (Anosys) | IRX-2 (Immuno-Rx) |
| | oncophage (Antigenics) | BLP-25 (Biomira) |
| | pentrix (Australian Cancer | PEP-005 (Peplin Biotech) |
| | Technology) | MGV (Progenics) |
| | GMK (Progenics) | synchrovax vaccines (CTL Immuno) |
| | ISF-154 (Tragen) | beta.-alethine (Dovetail) |
| | adenocarcinoma vaccine (Biomira) | melanoma vaccine (CTL Immuno) |
| | cancer vaccine (Intercell) | CLL therapy (Vasogen) |
| | CTP-37 (A VI BioPharma) | p21 RAS vaccine (GemVax) |
| Hormonal and | estrogens | bicalutamide |
| antihormonal | Prednisone | testosterone propionate; |
| agents | conjugated estrogens | fluoxymesterone |
| | methylprednisolone | flutamide |
| | ethinyl estradiol | methyltestosterone |
| | prednisolone | octreotide |
| | chlortrianisen | diethylstilbestrol |
| | aminoglutethimide | nilutamide |
| | idenestrol | megestrol |
| | leuprolide | mitotane tamoxifen |
| | hydroxyprogesterone caproate | P-04 (Novogen) |
| | goserelin | Toremofine |
| | medroxyprogesterone | 2-methoxyestradiol (EntreMed) |
| | leuporelin | dexamethasone |
| | testosterone | arzoxifene (Eli Lilly) |
| Photodynamic | talaporfin (Light Sciences) | motexafin |
| agents | Pd-bacteriopheophorbide (Yeda) | gadolinium (Pharmacyclics) |
| | Theralux (Theratechnologies) | hypericin |
| | lutetium texaphyrin (Pharmacyclics) | |
| Tyrosine Kinase | imatinib (Novartis) | C225 (ImClone) |
| Inhibitors | kahalide F (PharmaMar) | ZD4190 (AstraZeneca) |
| | leflunomide (Sugen/Pharmacia) | rhu-Mab (Genentech) |
| | CEP-701 (Cephalon) | ZD6474 (AstraZeneca) |
| | ZD1839 (AstraZeneca) | MDX-H210 (Medarex) |
| | CEP-751 (Cephalon) | vatalanib (Novartis) |
| | erlotinib (Oncogene Science) | 2C4 (Genentech) |
| | MLN518 (Millenium) | PKI166 (Novartis) |
| | canertinib (Pfizer) | MDX-447 (Medarex) |
| | PKC412 (Novartis) | GW2016 (GlaxoSmithKline) |
| | squalamine (Genaera) | ABX-EGF (Abgenix) |
| | phenoxodiol ( ) | EKB-509 (Wyeth) |
| | SU5416 (Pharmacia) | IMC-1C11 (ImClone) |
| | trastuzumab (Genentech) | EKB-569 (Wyeth) |
| | SU6668 (Pharmacia) | |

Miscellaneous agents

| | |
|---|---|
| SR-27897 (CCK A inhibitor, Sanofi- | gemtuzumab (CD33 antibody, Wyeth Ayerst) |
| Synthelabo) | CCI-779 (mTOR kinase inhibitor, Wyeth) |
| BCX-1777 (PNP inhibitor, BioCryst) | PG2 (hematopoiesis enhancer, Pharmagenesis) |
| tocladesine (cyclic AMP agonist, Ribapharm) | exisulind (PDE V inhibitor, Cell Pathways) |
| ranpirnase (ribonuclease stimulant, Alfacell) | Immunol ™ (triclosan oral rinse, Endo) |
| alvocidib (CDK inhibitor, Aventis) | CP-461 (PDE V inhibitor, Cell Pathways) |
| galarubicin (RNA synthesis inhibitor, Dong-A) | triacetyluridine (uridine prodrug, Wellstat) |
| CV-247 (COX-2 inhibitor, Ivy Medical) | AG-2037 (GART inhibitor, Pfizer) |
| tirapazamine (reducing agent, SRI International) | SN-4071 (sarcoma agent, Signature BioScience) |
| P54 (COX-2 inhibitor, Phytopharm) | WX-UK1 (plasminogen activator inhibitor, |
| N-acetylcysteine (reducing agent, Zambon) | Wilex) |
| CapCell ™ (CYP450 stimulant, Bavarian | TransMID-107 .TM. (immunotoxin, KS |
| Nordic) | Biomedix) |
| R-flurbiprofen (NF-kappaB inhibitor, Encore) | PBI-1402 (PMN stimulant, ProMetic |
| GCS-100 (gal3 antagonist, GlycoGenesys) | LifeSciences) |
| 3CPA (NF-kappaB inhibitor, Active Biotech) | PCK-3145 (apoptosis promotor, Procyon) |
| G17DT immunogen (gastrin inhibitor, Aphton) | bortezomib (proteasome inhibitor, Millennium) |
| seocalcitol (vitamin D receptor agonist, Leo) | doranidazole (apoptosis promotor, Pola) |
| efaproxiral (oxygenator, Allos Therapeutics) | SRL-172 (T cell stimulant, SR Pharma) CHS- |
| 131-I-TM-601 (DNA antagonist, | 828 (cytotoxic agent, Leo) |
| TransMolecular) | TLK-286 (glutathione S transferase inhibitor, |
| PI-88 (heparanase inhibitor, Progen) | Telik) |
| eflornithine (ODC inhibitor, ILEX Oncology) | trans-retinoic acid (differentiator, NIH) |
| tesmilifene (histamine antagonist, YM | PT-100 (growth factor agonist, Point |
| BioSciences) | Therapeutics) |
| minodronic acid (osteoclast inhibitor, | MX6 (apoptosis promotor, MAXIA) |
| Yamanouchi) | midostaurin (PKC inhibitor, Novartis) |
| histamine (histamine H2 receptor agonist, | apomine (apoptosis promotor, ILEX Oncology) |
| Maxim) | bryostatin-1 (PKC stimulant, GPC Biotech) |
| indisulam (p53 stimulant, Eisai) | urocidin (apoptosis promotor, Bioniche) |
| tiazofurin (IMPDH inhibitor, Ribapharm) | CDA-II (apoptosis promotor, Everlife) |
| aplidine (PPT inhibitor, PharmaMar) | Ro-31-7453 (apoptosis promotor, La Roche) |
| cilengitide (integrin antagonist, Merck KGaA) | SDX-101 (apoptosis promotor, Salmedix) |
| rituximab (CD20 antibody, Genentech) | brostallicin (apoptosis promotor, Pharmacia) |
| SR-31747 (IL-1 antagonist, Sanofi-Synthelabo) | ceflatonin (apoptosis promotor, ChemGenex) |

Additional combinations may also include agents which reduce the toxicity of the aforesaid agents, such as hepatic toxicity, neuronal toxicity, nephrotoxicity and the like.

### Screening Assays

The compounds of the present invention may also be used in a method to screen for other compounds that bind to an IAP BIR domain. Generally speaking, to use the compounds of the invention in a method of identifying compounds that bind to an IAP BIR domain, the IAP is bound to a support, and a compound of the invention is added to the assay. Alternatively, the compound of the invention may be bound to the support and the IAP is added.

There are a number of ways in which to determine the binding of a compound of the present invention to the BIR domain. In one way, the compound of the invention, for example, may be fluorescently or radioactively labeled and binding determined directly. For example, this may be done by attaching the IAP to a solid support, adding a detectably labeled compound of the invention, washing off excess reagent, and determining whether the amount of the detectable label is that present on the solid support. Numerous blocking and washing steps may be used, which are known to those skilled in the art.

In some cases, only one of the components is labeled. For example, specific residues in the BIR domain may be labeled. Alternatively, more than one component may be labeled with different labels; for example, using I¹²⁵ for the BIR domain, and a fluorescent label for the probe.

The compounds of the invention may also be used as competitors to screen for additional drug candidates or test compounds. As used herein, the terms “drug candidate” or “test compounds” are used interchangeably and describe any molecule, for example, protein, oligopeptide, small organic molecule, polysaccharide, polynucleotide, and the like, to be tested for bioactivity. The compounds may be capable of directly or indirectly altering the IAP biological activity.

Drug candidates can include various chemical classes, although typically they are small organic molecules having a molecular weight of more than 100 and less than about 2,500 Daltons. Candidate agents typically include functional groups necessary for structural interaction with proteins, for example, hydrogen bonding and lipophilic binding, and typically include at least an amine, carbonyl, hydroxyl, ether, or carboxyl group. The drug candidates often include cyclical carbon or heterocyclic structures and/or aromatic or polyaromatic structures substituted with one or more functional groups.

Drug candidates can be obtained from any number of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means.

Competitive screening assays may be done by combining an IAP BIR domain and a probe to form a probe:BIR domain complex in a first sample followed by adding a test compound from a second sample. The binding of the test is determined, and a change or difference in binding between the two samples indicates the presence of a test compound capable of binding to the BIR domain and potentially modulating the IAP's activity.

In one case, the binding of the test compound is determined through the use of competitive binding assays. In this embodiment, the probe is labeled with a fluorescent label. Under certain circumstances, there may be competitive binding between the test compound and the probe. Test compounds which display the probe, resulting in a change in fluorescence as compared to control, are considered to bind to the BIR region.

In one case, the test compound may be labeled. Either the test compound, or a compound of the present invention, or both, is added first to the IAP BIR domain for a time sufficient to allow binding to form a complex.

Formation of the probe:BIR domain complex typically require Incubations of between 4° C. and 40° C. for between 10 minutes to about 1 hour to allow for high-throughput screening. Any excess of reagents are generally removed or washed away. The test compound is then added, and the presence or absence of the labeled component is followed, to indicate binding to the BIR domain.

In one case, the probe is added first, followed by the test compound. Displacement of the probe is an indication the test compound is binding to the BIR domain and thus is capable of binding to, and potentially modulating, the activity of IAP. Either component can be labeled. For example, the presence of probe in the wash solution indicates displacement by the test compound. Alternatively, if the test compound is labeled, the presence of the probe on the support indicates displacement.

In one case, the test compound may be added first, with incubation and washing, followed by the probe. The absence of binding by the probe may indicate the test compound is bound to the BIR domain with a higher affinity. Thus, if the probe is detected on the support, coupled with a lack of test compound binding, may indicate the test compound is capable of binding to the BIR domain.

Modulation is tested by screening for a test compound's ability to modulate the activity of IAP and includes combining a test compound with an IAP BIR domain, as described above, and determining an alteration in the biological activity of the IAP. Therefore in this case, the test compound should both bind to the BIR domain (although this may not be necessary), and alter its biological activity as defined herein.

Positive controls and negative controls may be used in the assays. All control and test samples are performed multiple times to obtain statistically significant results. Following incubation, all samples are washed free of non-specifically bound material and the amount of bound probe determined. For example, where a radiolabel is employed, the samples may be counted in a scintillation counter to determine the amount of bound compound.

Typically, the signals that are detected in the assay may include fluorescence, resonance energy transfer, time resolved fluorescence, radioactivity, fluorescence polarization, plasma resonance, or chemiluminescence and the like, depending on the nature of the label. Detectable labels useful in performing screening assays in this invention include a fluorescent label such as Fluorescein, Oregon green, dansyl, rhodamine, tetramethyl rhodamine, texas red, Eu³⁺; a chemiluminescent label such as luciferase; colorimetric labels; enzymatic markers; or radioisotopes such as tritium, I¹²⁵ and the like

Affinity tags, which may be useful in performing the screening assays of the present invention include be biotin, polyhistidine and the like.

### Synthesis and Methodology

General methods for the synthesis of the compounds of the present invention are shown below and are disclosed merely for the purpose of illustration and are not meant to be interpreted as limiting the processes to make the compounds by any other methods. Those skilled in the art will readily appreciate that a number of methods are available for the preparation of the compounds of the present invention.

### General Procedures

Several methods for preparing symmetrically or non-symmetrically bridged compounds represented by Formula 1 may be envisioned. General methods are illustrated in Schemes 1, 2, 3 and 4.

Scheme 1 illustrates a general procedure for the preparation of intermediate 1-iv. Activation of the carboxylic acid of (S)-4-(Benzyloxycarbonyl)-1-(tert-butoxycarbonyl)piperazine-2-carboxylic acid with peptide coupling agents and treatment with a primary or secondary amine, and deprotection of PG² provides the amide intermediate 1-i. Peptide coupling of PG³(H)N(R³)CHCO₂H with 1-i is effected by activation of the carboxylic acid of PG³(H)N(R³)CHCO₂H with peptide coupling agents, followed by the addition of 1-i to provide the fully protected amide, which may be further deprotected at PG³ to provide intermediate 1-ii. Activation of the carboxylic acid of PG⁴(R¹)N(R²)CHCO₂H with peptide coupling agents, followed by the addition of 1-ii to provide intermediate 1-iii Deprotection of PG¹ provides intermediate 1-iv.

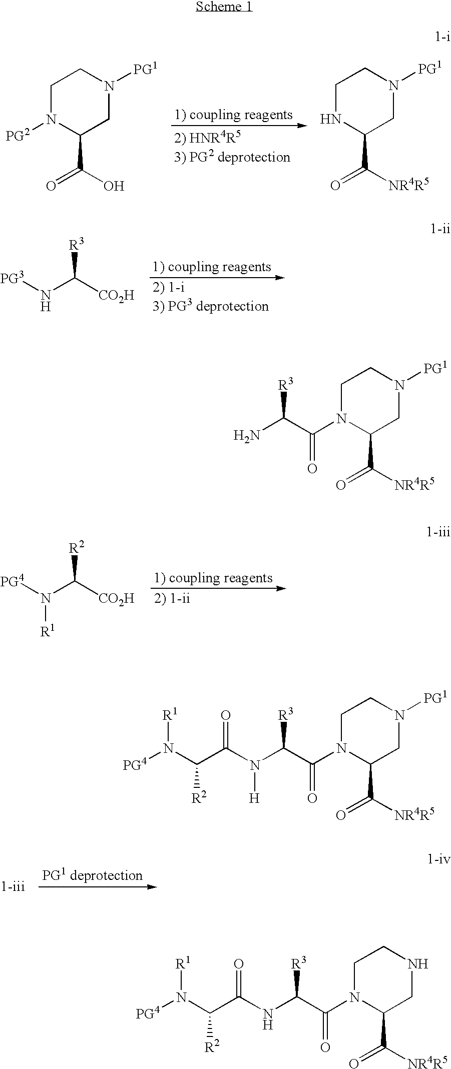

Bridging of intermediate 1-iv can be accomplished by the treatment of intermediate 1-iv with an activated diacid, as shown in Scheme 2. Deprotection of PG⁴ yields compounds of formula 2-ii.

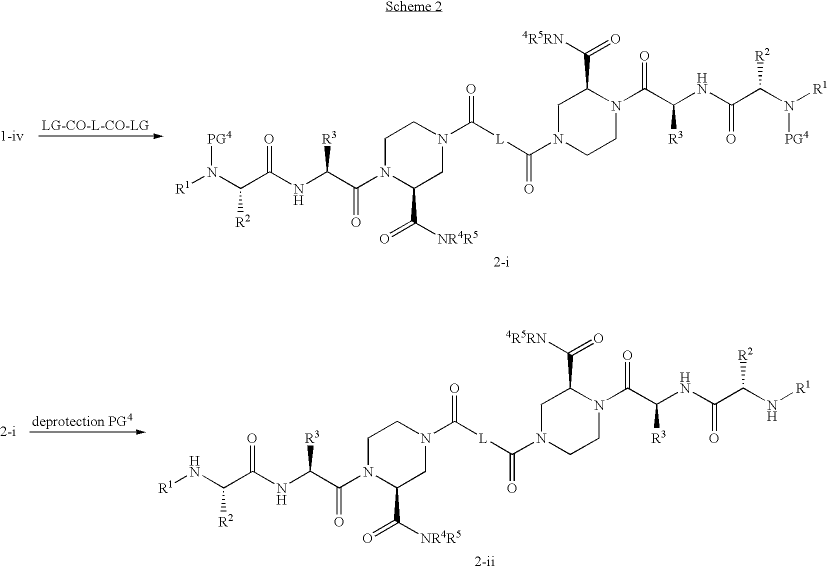

Bridging of intermediate 1-iv can be accomplished by the treatment of intermediate 1-iv with a bis-sulfonyl halide, as shown in Scheme 3. Deprotection of PG⁴ yields compounds of formula 3-ii.

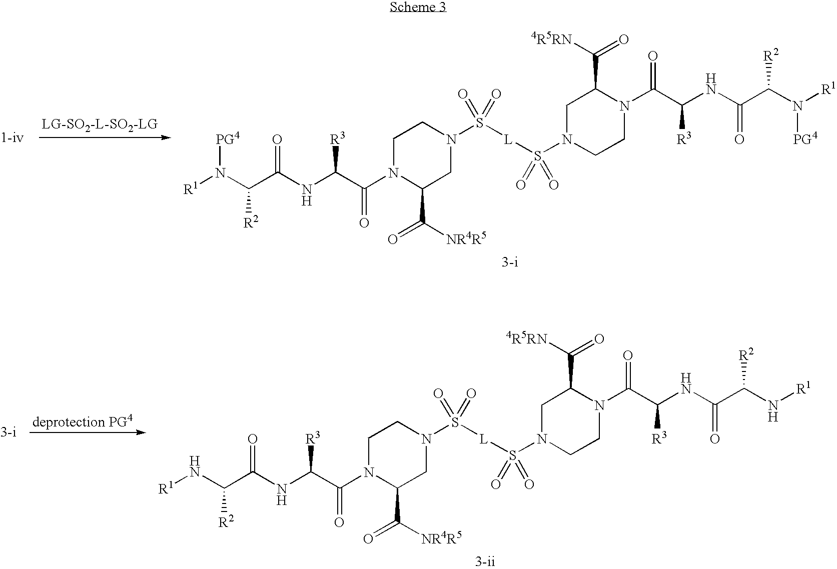

Scheme 4 illustrates the use of functionalized amino acids as bridging groups. PG⁵(H)N(R⁶)CHCO₂H is coupled to intermediate 1-iv using amino acid coupling reagents, followed by deprotection of PG⁵ yields intermediate 4-i. Treatment of 4-i with LG-(O)C-L-C(O)-LG followed by deprotection of PG⁴ yields compounds of formula 4-ii.

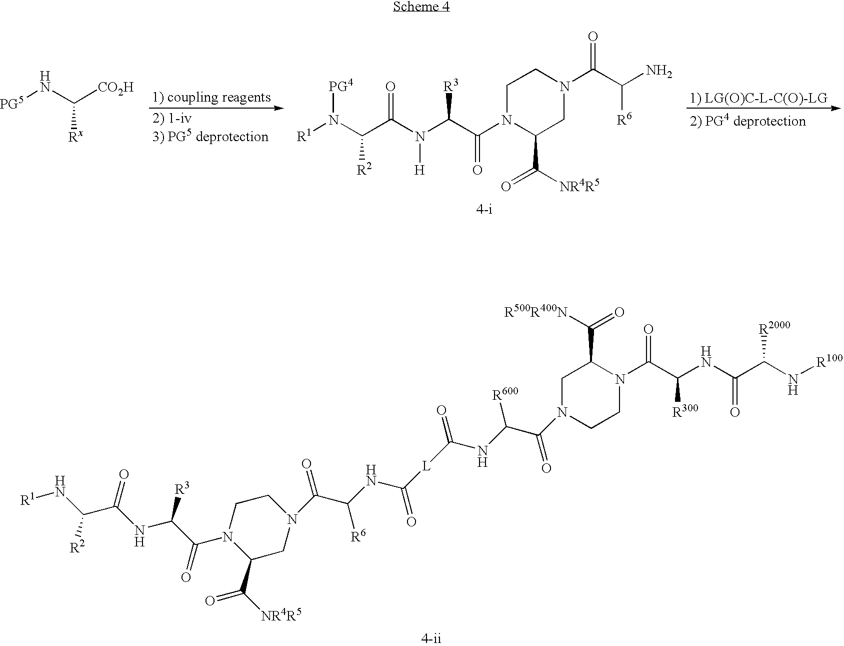

The above Schemes are applicable to both symmetrical compounds and unsymmetrical compounds of the present invention. The substituents R¹, R¹⁰⁰, R², R²⁰⁰, R³, R³⁰⁰, R⁴, R⁴⁰⁰, R⁵, R⁵⁰⁰ and the like are as defined herein. LG is a leaving group such as, for example Cl, Br, I, OTs, OSu or OMs.

A similar process as that depicted in Scheme 4 can be applied to the synthesis or sulfonamide bridged compounds.

### Examples

The following abbreviations are used throughout:

Boc: t-butoxycarbonyl;
CBz: benzyloxycarbonyl;
DCM: dichloromethane, CH₂Cl₂;
DIPEA: diisopropylethylamine;
DMAP: 4-(dimethylamino)pyridine;

### DMF: N,N-dimethylformamide;

DTT: dithiothreitol;
EDC: 3-dimethylaminopropyl-3-ethylcarbodiimide hydrochloride;
EDTA: ethylenediaminetetraacetic acid;
Fmoc: N-(9-fluorenylmethoxycarbonyl);
HBTU: O-(benzotriazol-1-yl)-N,N,N,N′,N′-tetramethyluronium hexafluorophosphate;
HCl: hydrochloric acid;
HOAc: acetic acid;
HOBt: 1-hydroxybenzotriazole;
HPLC: high performance liquid chromatography;
LCMS: liquid chromatography-mass spectrometer;
MeOH: methanol;
MgSO₄: magnesium sulfate;
MS: mass spectrum;
NaHCO₃: sodium hydrogen carbonate;
Pd/C: palladium on carbon;
TEA: triethylamine;
THF: tetrahydrofuran; and

### TMEDA: N,N,N,N-tetramethylethylenediamine.

### Synthetic Methods

The following section summarizes synthetic methods used in the synthesis of compounds of the instant invention.

The preparation of intermediate 5-f is illustrated in scheme 5. The conversion of intermediate 5-f to compounds 1 and 12 is summarized in schemes 6 and 7. The conversion of intermediate 5-f to intermediate 8-b is illustrated in scheme 8. The conversion of intermediate 8-b to compound II is summarized in scheme 9.

### Synthesis of Intermediate 5-f

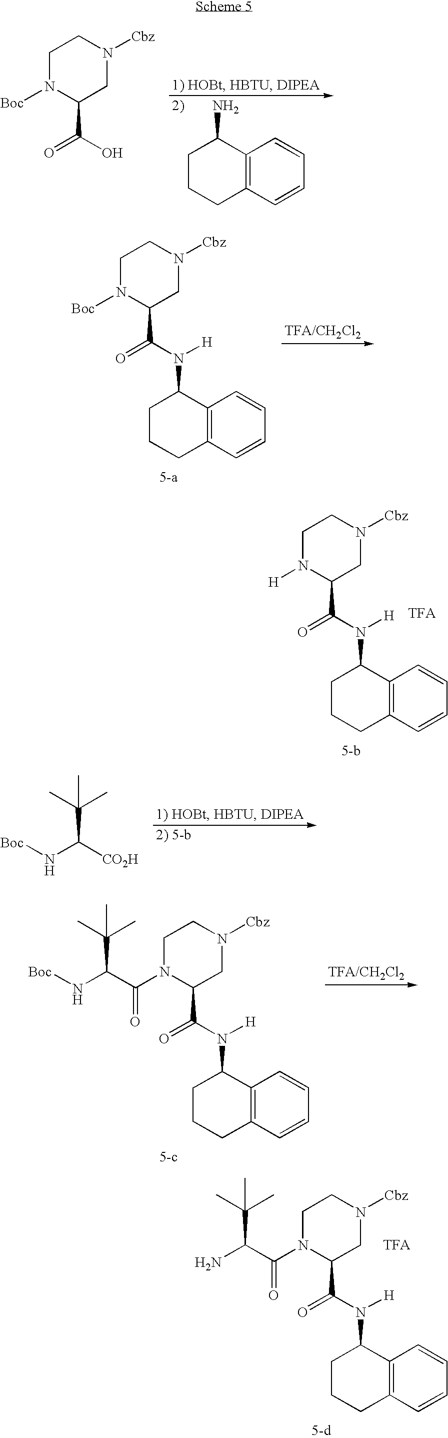

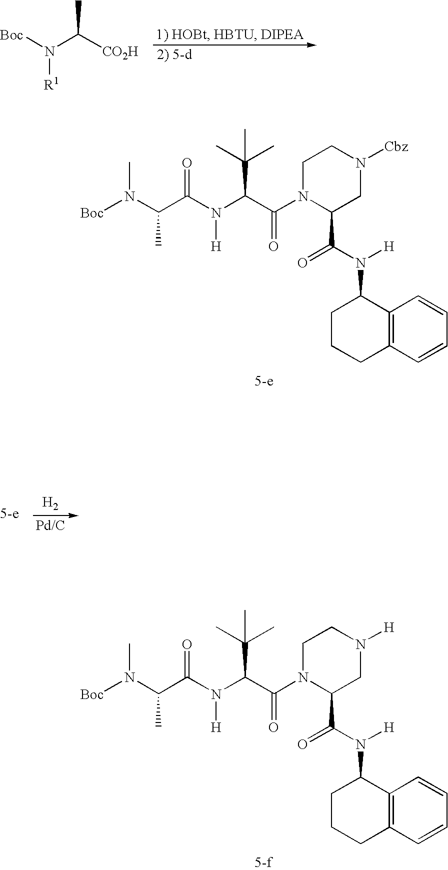

### Step 1:

(S)-4-(Benzyloxycarbonyl)-1-(tert-butoxycarbonyl)piperazine-2-carboxylic acid (1.00 g, 2.70 mmol), HOBt (520 mg, 3.80 mmol), HBTU (1.35 g, 3.60 mmol) and DIPEA (955 uL, 5.50 mmol) were dissolved in dry DMF (10 ml) under N₂ and stirred for 10 minutes at room temperature. R-1,2,3,4-Tetrahydro-1-napthylamine (485 mg, 3.30 mmol) was added and the solution was left to stir for 18 hours at room temperature. The contents were then added to a separatory funnel along with EtOAc and washed with 10% citric acid, saturated NaHCO₃ and brine. The organic layer was collected, dried over anhydrous MgSO₄, filtered and concentrated under reduced pressure to provide intermediate 5-a as a white solid.

### Step 2:

Intermediate 5-a was dissolved in dichloromethane (5 mL) and trifluoroacetic acid (5 mL) was added and the solution stirred for 1 hour at room temperature. Volatiles were removed under reduced pressure and the residue was triturated with diethyl ether to provide intermediate 5-b as a white solid. MS (m/z) M+1=394.2

### Step 3:

Boc-Tle-OH (443 mg, 1.9 mmol) HOBt (302 mg, 2.2 mmol), HBTU (796 mg, 2.1 mmol) and DIPEA (835 uL, 2.2 mmol) were dissolved in dry DMF (10 ml) under N₂ and stirred for 10 minutes at room temperature. Intermediate 5-b (810 mg, 1.6 mmol) was then added and the solution was left to stir for 18 hours at room temperature. The contents were then added to a separatory funnel along with EtOAc and washed with 10% citric acid, saturated NaHCO₃ and brine. The organic layer was collected dried over anhydrous MgSO₄, filtered and concentrated in vacuo. Purification by silica gel chromatography eluting with a hexane/tetrahydrofuran gradient provided intermediate 5-c as a white solid.

### Step 4:

Intermediate 5-c (510 mg) was dissolved in dichloromethane (3 mL) and trifluoroacetic acid (3 mL) was added and the solution stirred for 1 hour at room temperature. Volatiles were removed under reduced pressure and the residue was triturated with diethyl ether to provide intermediate 5-d as a white solid. MS (m/z) M+1=507.4

### Step 5:

Boc-NMe-OH (114 mg, 0.56 mmol), HOBt (89 mg, 1.4 mmol), HBTU (232 mg, 0.61 mmol) and DIPEA (327 uL, 1.9 mmol) were dissolved in dry DMF (5 ml) under N₂ and stirred for 10 min at room temperature. Intermediate 5-d (290 mg, 0.47 mmol) was then added and the solution was left to stir for 18 hours at room temperature. The contents were then added to a separatory funnel along with EtOAc and washed with 10% citric acid, saturated NaHCO₃ and brine. The organic layer was collected dried over anhydrous MgSO₄, filtered and concentrated in vacuo. Purification by silica gel chromatography, eluting with a hexane/tetrahydrofuran gradient, provided intermediate 5-e as a white solid. MS (m/z) M+1=692.6

### Step 6:

To a solution of intermediate 5-e (160 mg, 0.23 mmol) in anhydrous MeOH and stirred under N₂ was added 10% Pd/C (30 mg). The reaction mixture was purged with H₂ and stirred for 24 hours at room temperature. The reaction was then filtered through celite and the filtrate was concentrated in vacuo to provide intermediate 5-f as a white solid. MS (m/z) M+1=558.4

### Synthesis of Compound 1

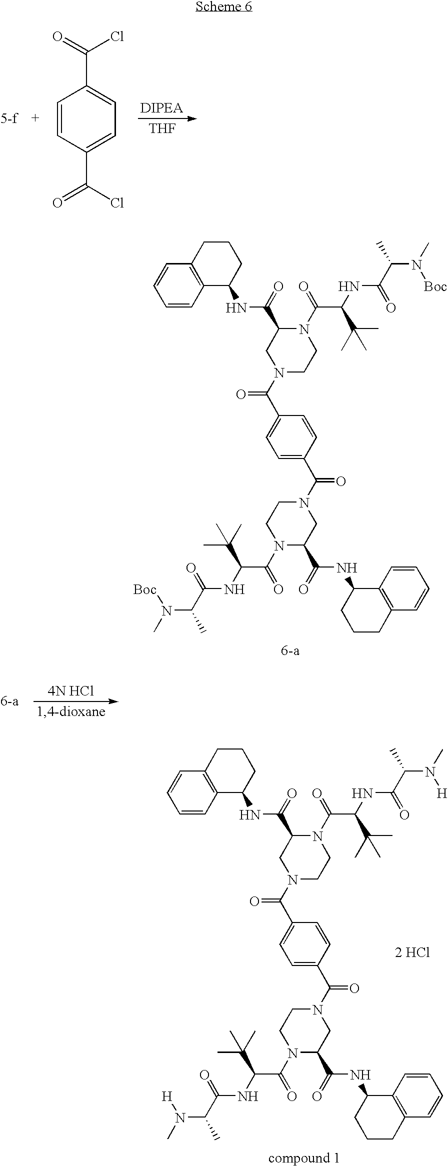

### Step 1:

Intermediate 5-f (50.0 mg, 0.090 mmol), teraphtaloyl chloride (10.0 mg, 0.045 mmol) and DIPEA (19 ul, 1.2 mmol) were dissolved in dichloromethane (5 mL) under N₂ at room temperature. DMAP (catalytic amount) was added and the solution was left to stir for 24 hours at room temperature. The contents were then added to a separatory funnel along with EtOAc and washed with 10% citric acid, saturated NaHCO₃ and brine. The organic layer was collected dried over anhydrous MgSO₄, filtered and concentrated in vacuo to provide intermediate 6-a as a white solid.

### Step 2:

4N HCl in 1,4-dioxane (1 mL) was added to intermediate 6-a (48 mg, 0.04 mmol) at 0° C. and the solution was stirred for 1 hour at 0° C. Volatiles were removed under reduced pressure and the residue was triturated with diethyl ether to provide the expected compound 1•2HCl as a white solid. MS (m/z) M+1=1045.6

### Synthesis of Compound 12

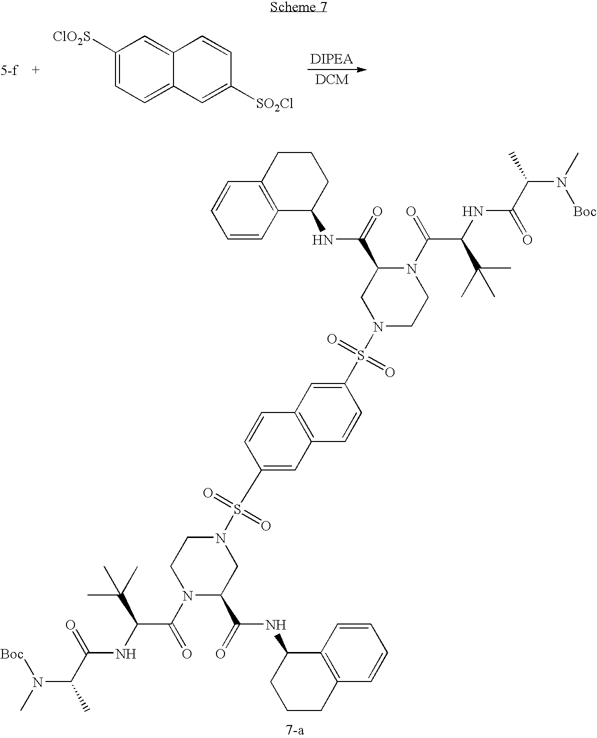

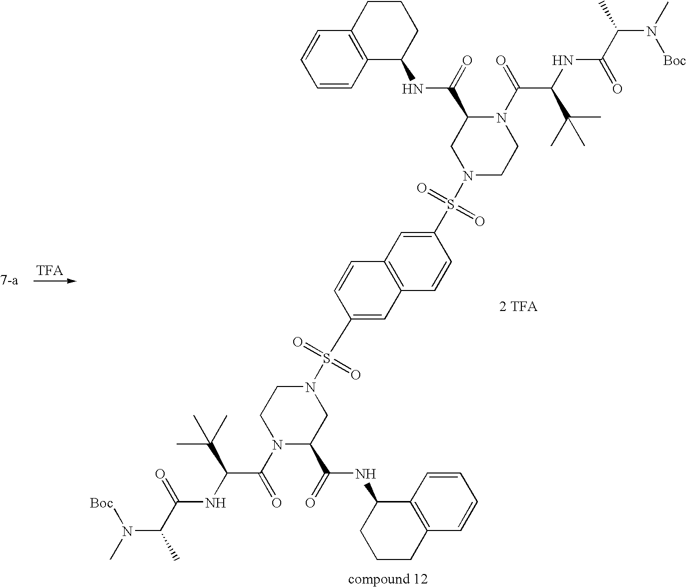

### Step 1

To a solution of Intermediate 5-f (200 mg, 0.35 mmol) in DCM cooled to 0° C. were sequentially added DIPEA (0.075 ml, 0.42 mmol) DMAP (cat) and naphthalene-2,6-disulfonyl dichloride (55 mg, 0.17 mmol) and the reaction was stirred overnight at room temperature. Water and ethyl acetate were added, the organic layer was separated, washed with 10% citric acid, aqueous NaHCO₃ and brine, dried over MgSO₄, filtered and concentrated in vacuo. Purification by silica gel chromatography eluting with a hexane/tetrahydrofuran gradient provided intermediate 7-a as a white solid.

### Step 2

To a solution of intermediate 7-a (121 mg, 0.088 mmol) in DCM (3 ml) cooled to 0° C. was added TFA (3 ml) and the reaction was stirred for 1 hour at 0° C. Volatiles were removed under reduced pressure and the residue was triturated with diethyl ether to provide compound 12•2TFA as a white solid. MS (m/z) M+1=1167.4

### Synthesis of Intermediate 8-b

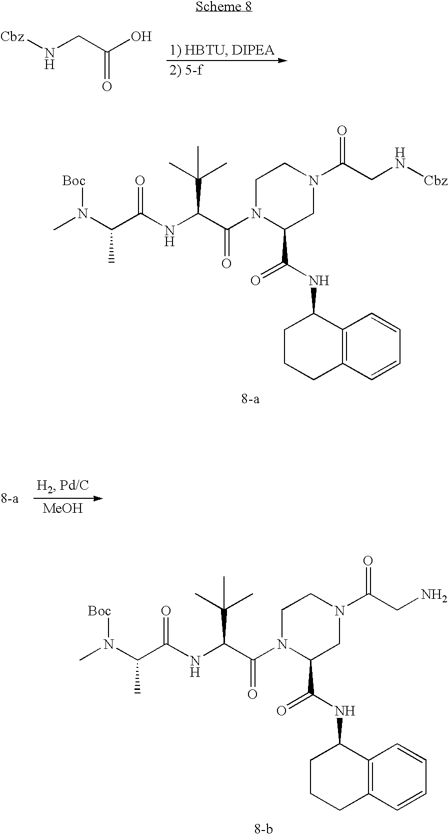

### Step 1

To a solution of Cbz-Gly-OH (450 mg, 2.15 mmol) in DMF cooled to 0° C. were sequentially added DIPEA (1.0 ml, 5.74 mmol), HOBt (5.74 ml, 3.44 mmol) and TFHH (455 mg, 1.72 mmol). After stirring for 30 minutes at 0° C., intermediate 5-f (800 mg, 1.43 mmol) was added and the reaction mixture was stirred for 4 hours at room temperature. Water and ethyl acetate were added, the organic layer was separated, washed with 10% citric acid, aqueous NaHCO₃ and brine, dried over MgSO₄, filtered and concentrated in vacuo. Purification by silica gel chromatography eluting with a hexane/tetrahydrofuran gradient provided the expected intermediate 8-a as a white solid.

### Step 2

To a solution of intermediate 8-a (1.8 g, 2.4 mmol) in anhydrous MeOH and stirred under N₂ was added 10% Pd/C (256 mg). The reaction mixture was purged with H₂ and stirred for 2 hours. The reaction was then filtered through celite and the filtrate was concentrated in vacuo to provide intermediate 8-b as a white solid. MS (m/z) M+1=615.4

### Synthesis of Compound 11

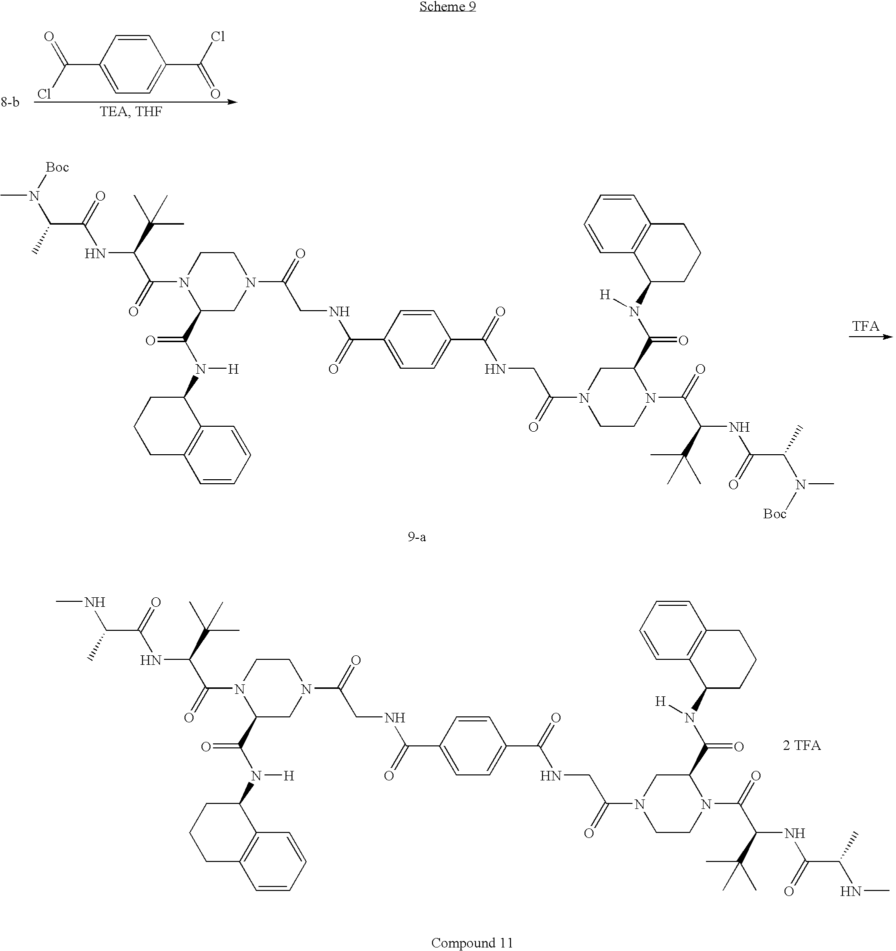

### Step 1

To a solution of intermediate 8-b (200 mg, 0.32 mmol) in DCM cooled to 0° C. were sequentially added DIPEA (68 ul, 0.38 mmol) and terephthaloyl dichloride (31 mg, 0.15 mmol) and the reaction was stirred for 6 hours at room temperature. Water and ethyl acetate were added, the organic layer was separated, washed with 10% citric acid, aqueous NaHCO₃ and brine, dried over MgSO₄, filtered and concentrated in vacuo. Purification by silica gel chromatography eluting with a hexane/tetrahydrofuran gradient provided intermediate 9-a as a white solid.

### Step 2

To a solution of intermediate 9-a (50 mg, 0.037 mmol) in DCM (3 ml) cooled to 0° C. was added TFA (3 ml) and the reaction was stirred for 1 hour at 0° C. Volatiles were removed under reduced pressure and the residue was triturated with diethyl ether to provide compound 11•2TFA as a white solid. MS (m/z) M+1=1159.6

Representative compounds of the present invention were prepared according to variations of the above procedures and are illustrated in Table 1:

TABLE 1

| COM- | | |
|---|---|---|
| POUND | STRUCTURE | MS |
| 1 | 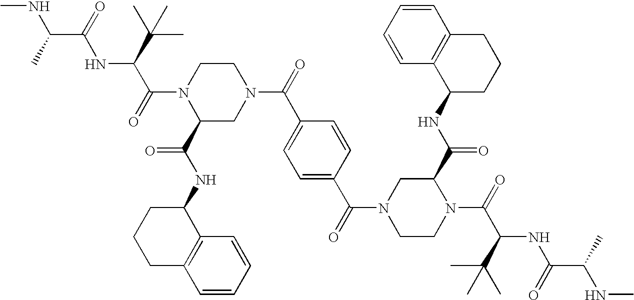 | M + 1 = 1045.6 |
| 2 | 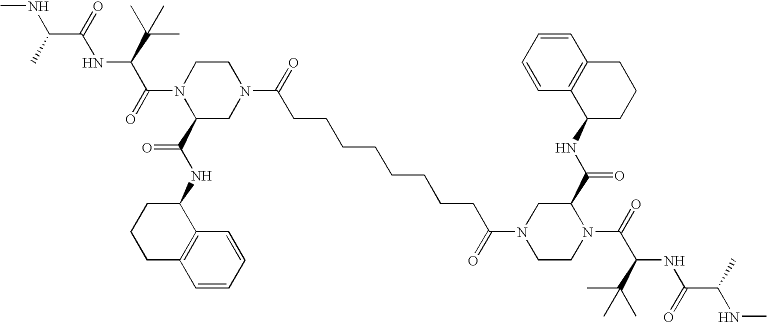 | M + 1 = 1081.6. |
| 3 | 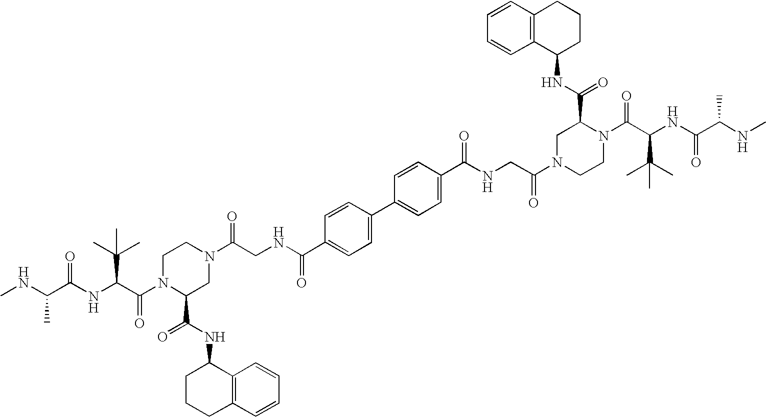 | M + 1 = 1235.6 |
| 4 | 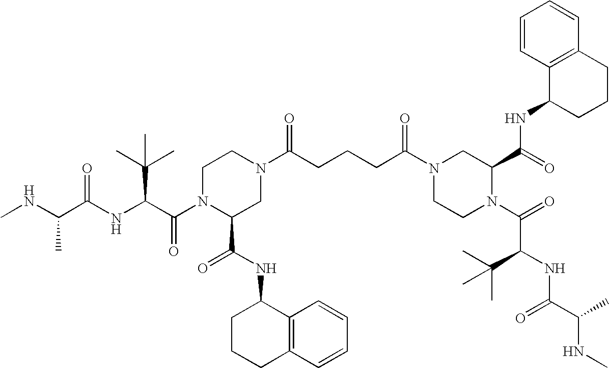 | M + 1 = 1011.6 |
| 5 | 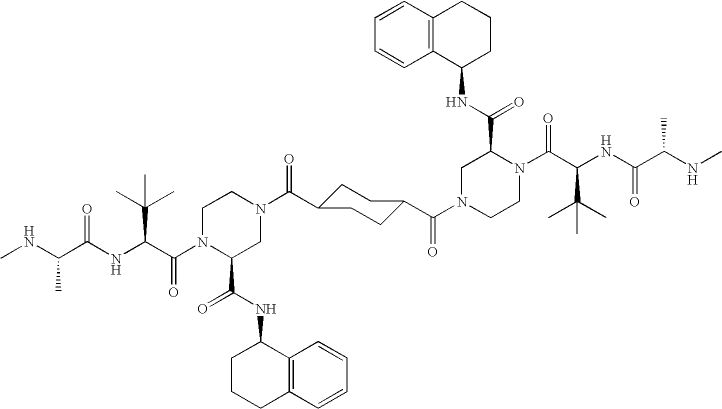 | M + 1 = 1051.6 |
| 6 | 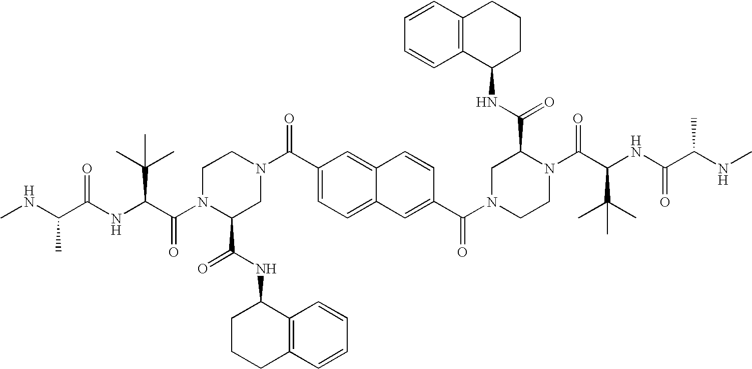 | M + 1 = 1095.6 |
| 7 | 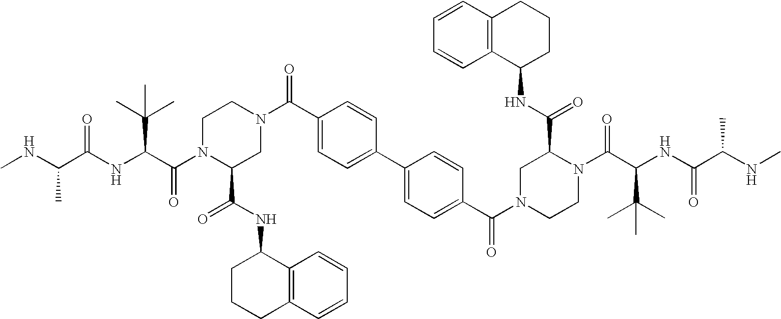 | M + 1 = 1121.6 |
| 8 | 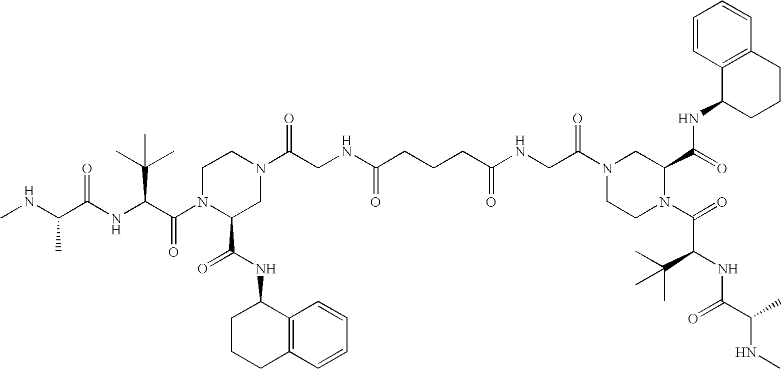 | M + 1 = 1125.6 |
| 9 | 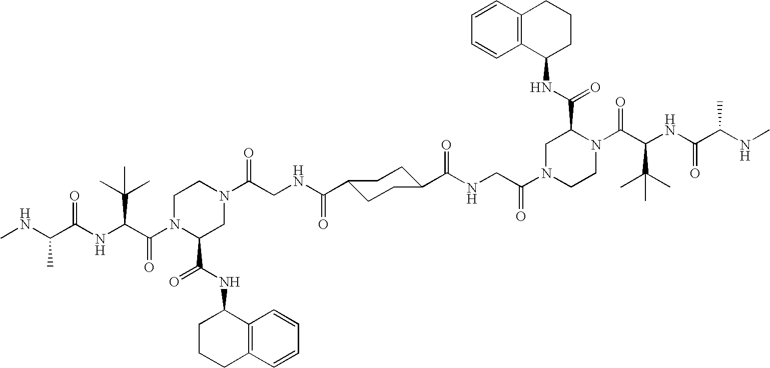 | M + 1 = 1165.6 |
| 10 | 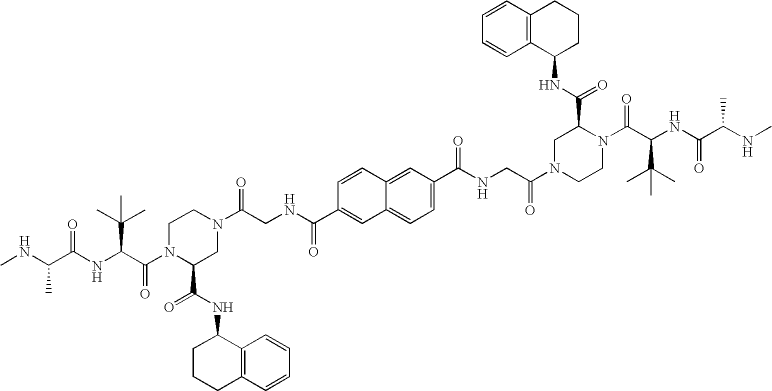 | M + 1 = 1209.6 |
| 11 | 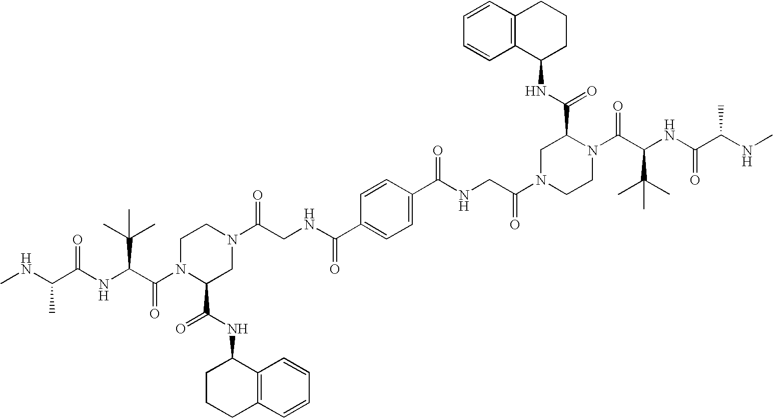 | M + 1 = 1159.6 |
| 12 | 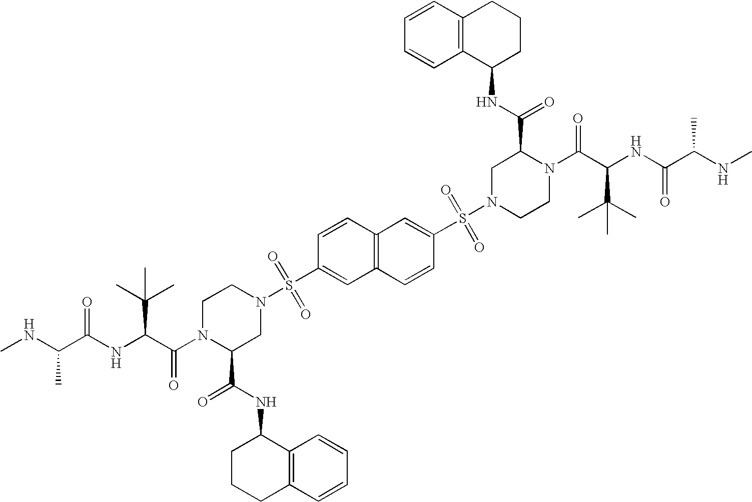 | M + 1 = 1167.4 |
| 13 | 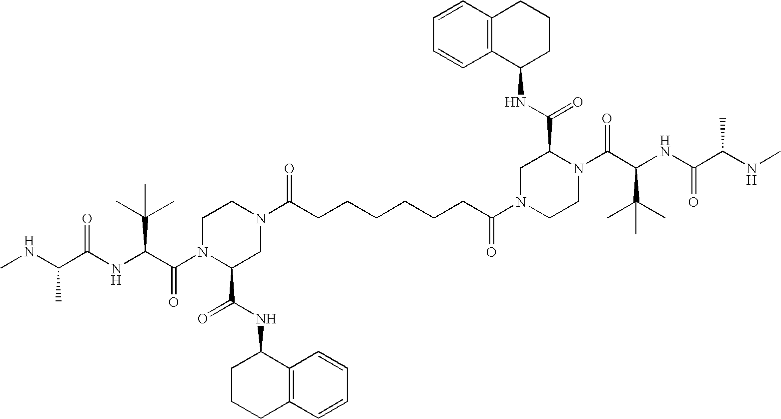 | M + 1 = 1053.6 |
| 14 | 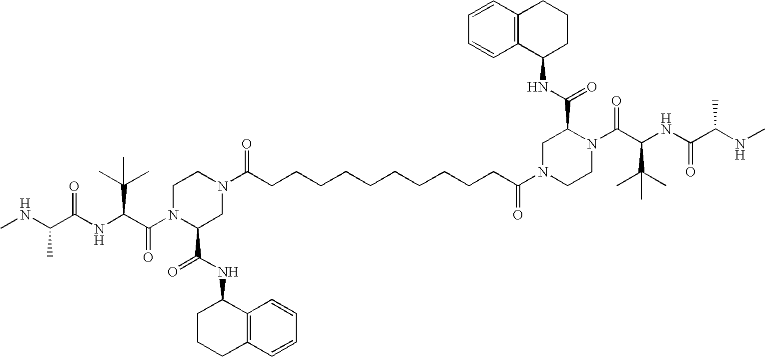 | M + 1 = 1109.6 |
| 15 | 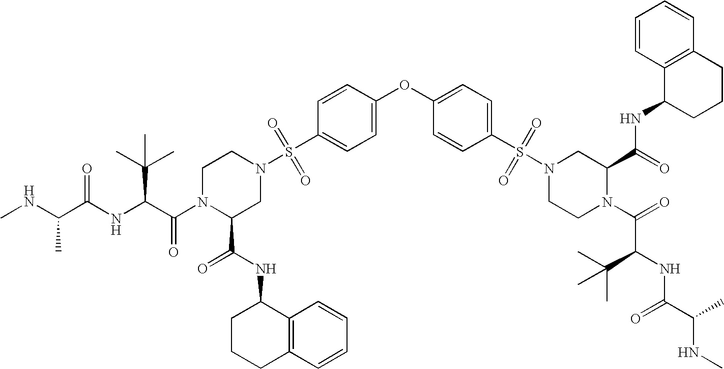 | M + 1 = 1209.4 |
| 16 | 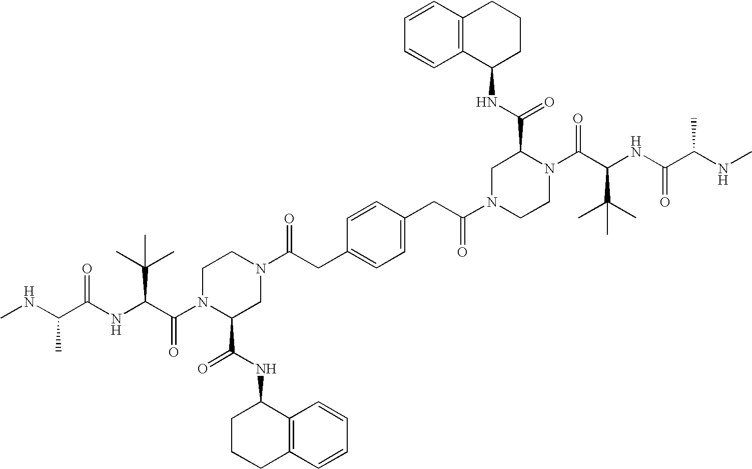 | M + 1 = 1073.6 |
| 17 | 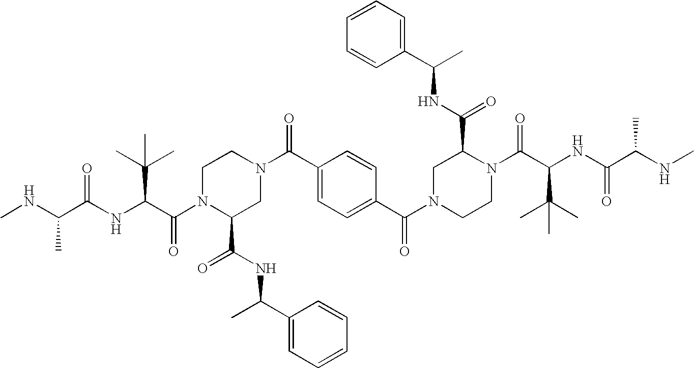 | M + 1 = 993.4 |
| 18 | 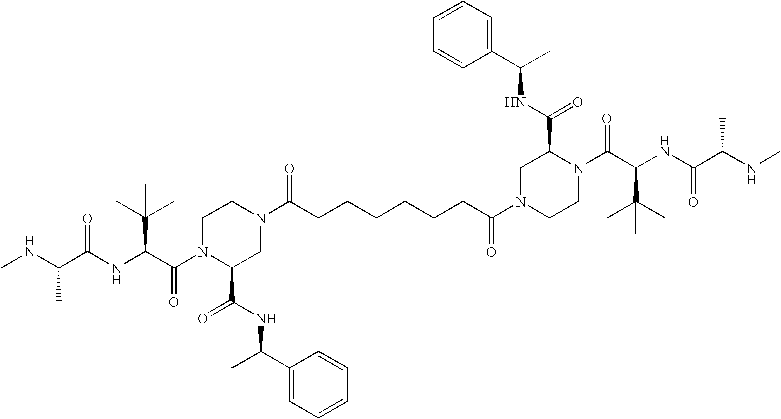 | M + 1 = 1001.5 |
| 19 | 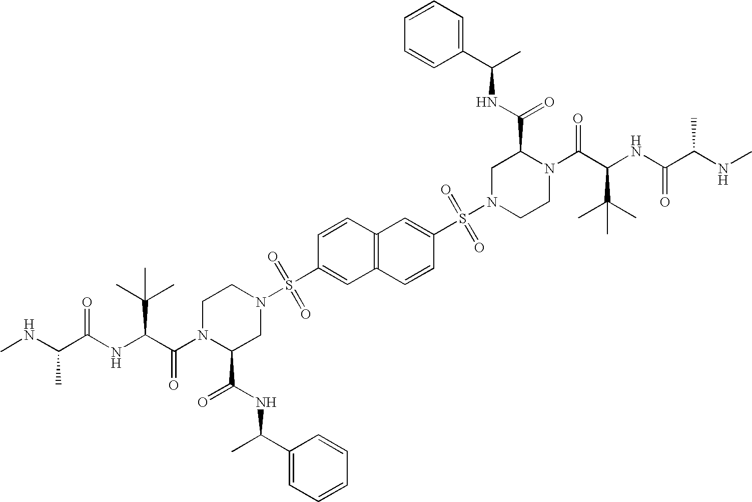 | M + 1 = 1115.4 |
| 20 | 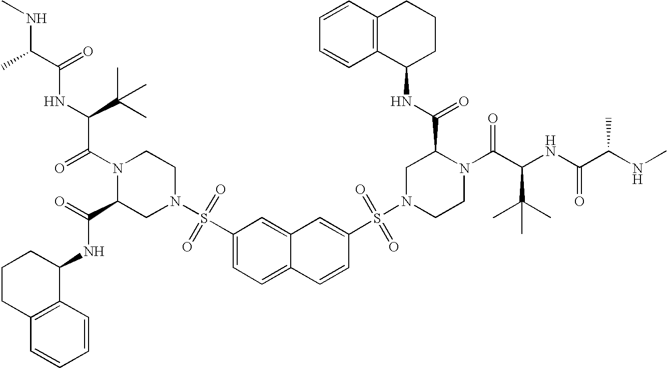 | M + 1 = 1166.4 |
| 21 | 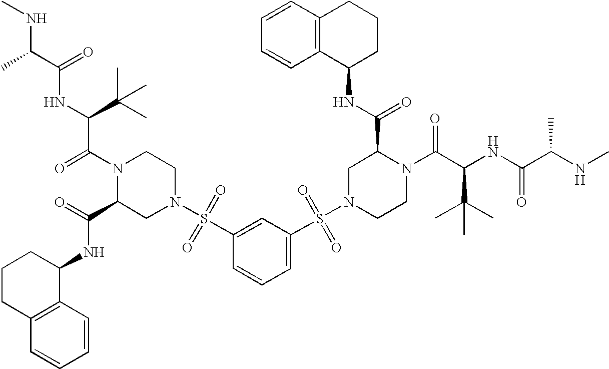 | M + 1 = 1117.4 |
| 22 | 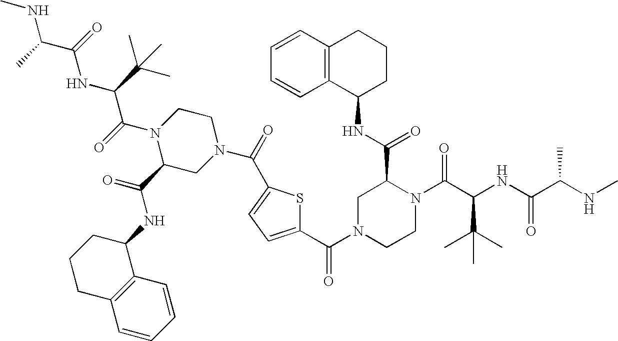 | M + 1 = 1051.4 |
| 23 | 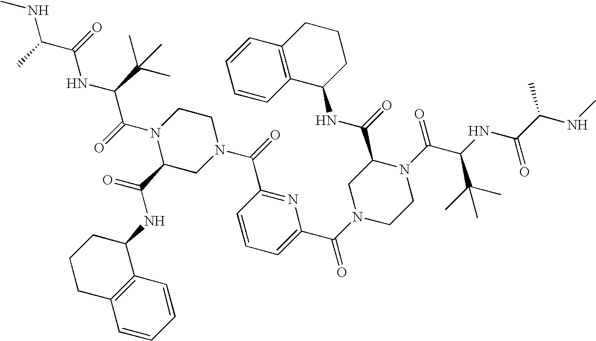 | M + 1 = 1046.4 |
| 24 | 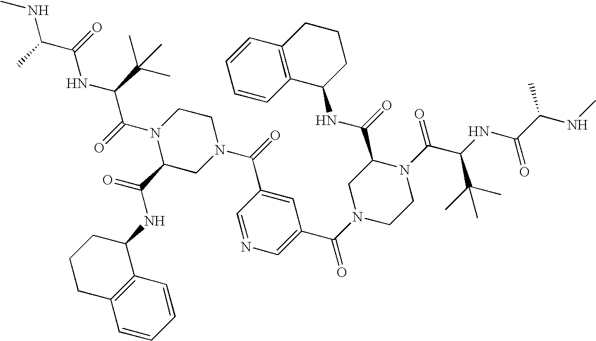 | M + 1 = 1046.4 |
| 25 | 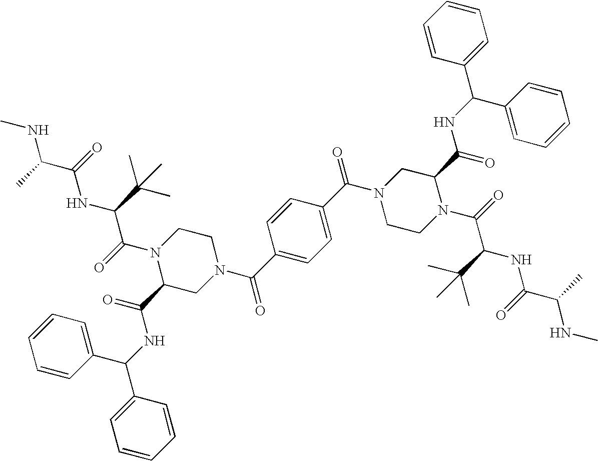 | M + 1 = 1117.4 |
| 26 | 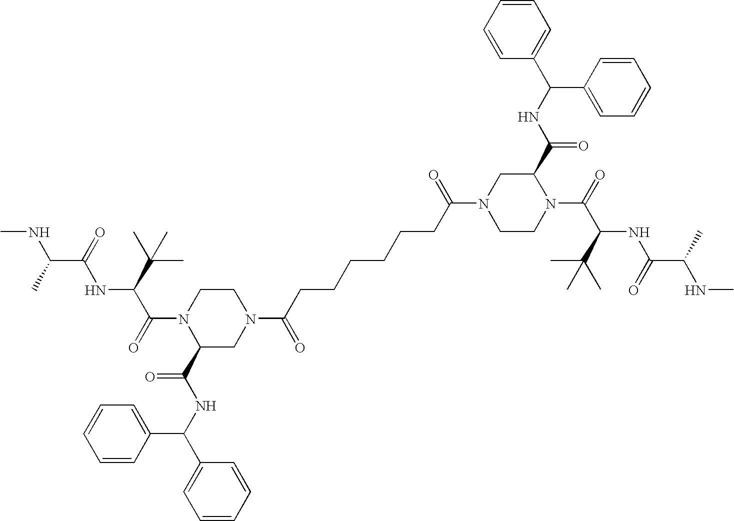 | M + 1 = 1125.6 |
| 27 | 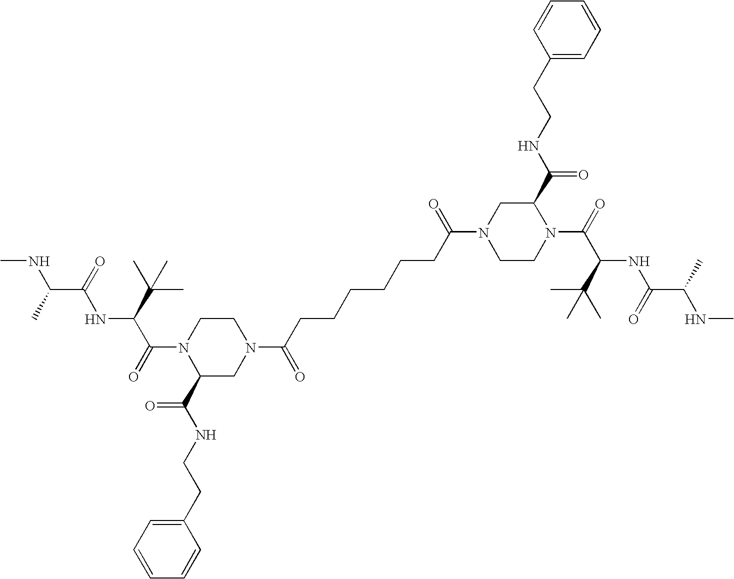 | M + 1 = 1001.2 |
| 28 | 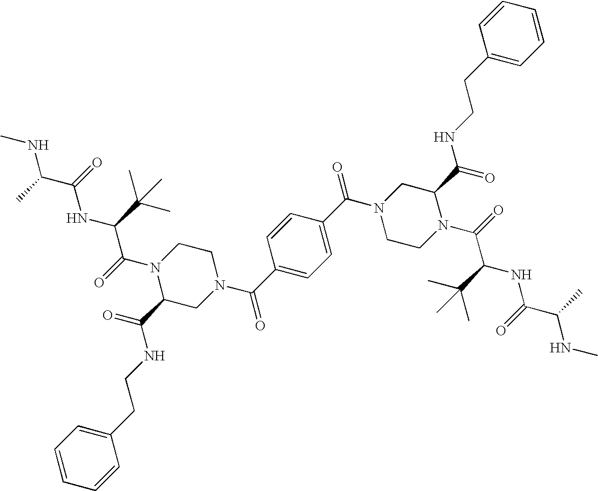 | M + 1 = 993.2 |

Representative compounds of the present invention which can be prepared by simple modification of the above procedures are illustrated in Tables 2 and 3:

TABLE 2

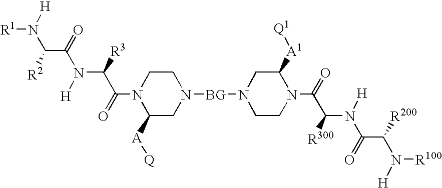

R¹, R², R³, R¹⁰⁰, R^{1a}, R^{100a}, R²⁰⁰ and R³⁰⁰ are defined as hereinabove,
BG is -X-L-X′-, and -X-L-X′- is chosen from:

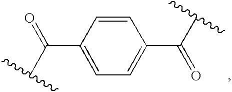

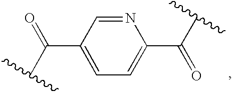

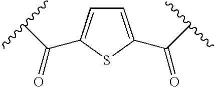

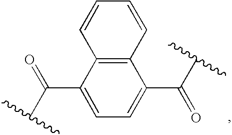

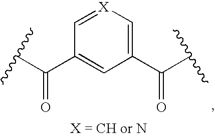

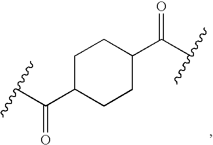

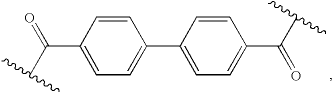

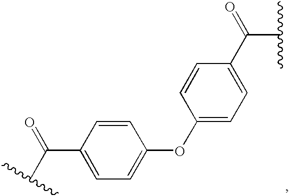

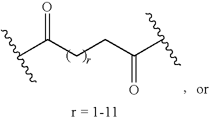

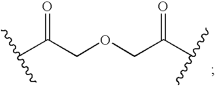

A = —CO—;
Q = NR⁴R⁵, wherein R⁴ and R⁴⁰⁰ and H and R⁵ and R⁵⁰⁰ and chosen from:

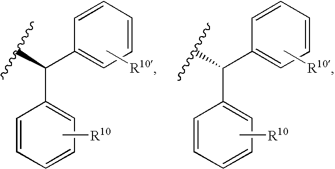

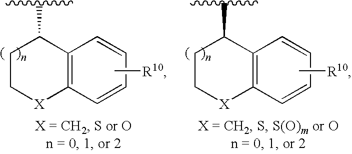

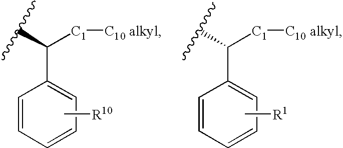

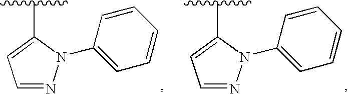

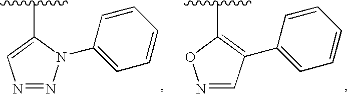

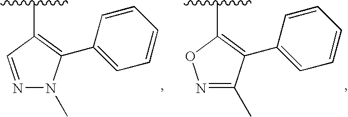

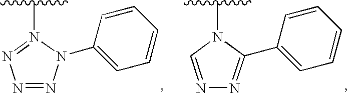

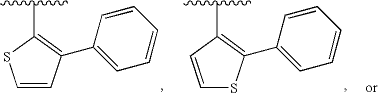

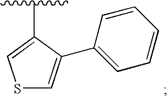

wherein the aryl moieties may be substituted by R¹⁰ and wherein,
R¹⁰ and R^{10′} are independently defined as R¹⁰ hereinabove, and
wherein the alkyl may be further substituted by R⁶ as
defined hereinabove.

TABLE 3
STRUCTURE

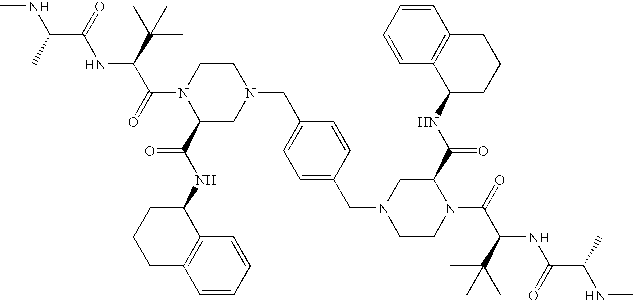

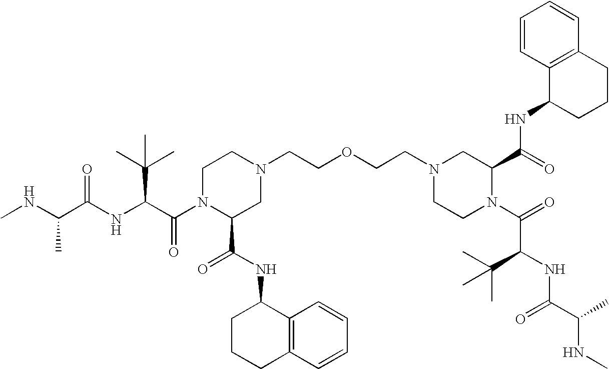

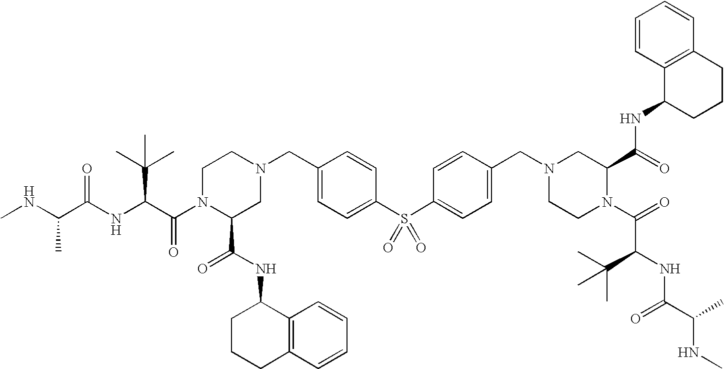

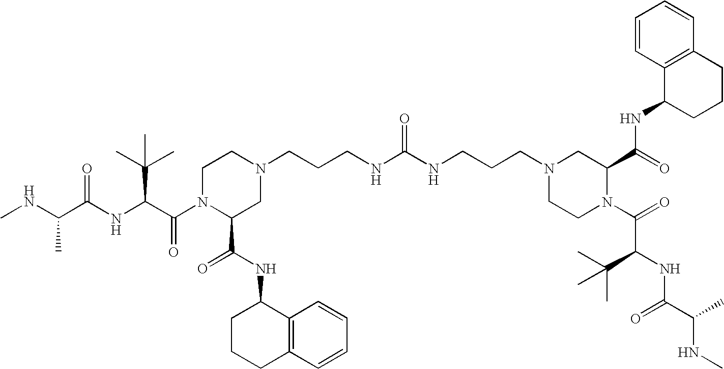

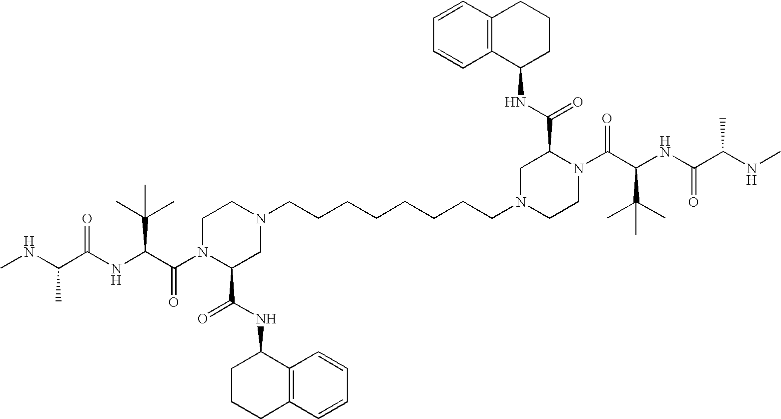

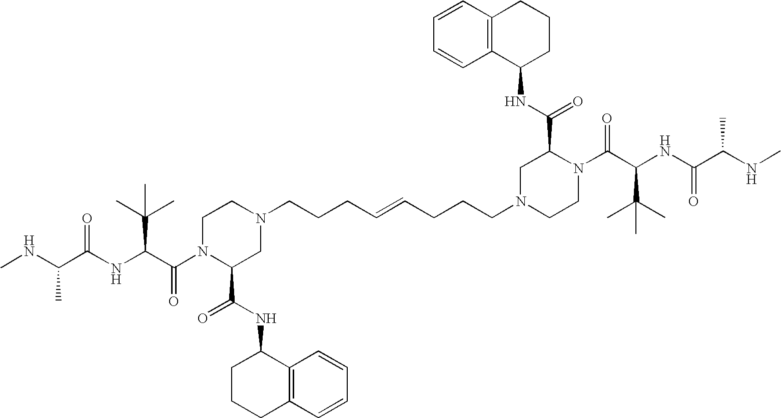

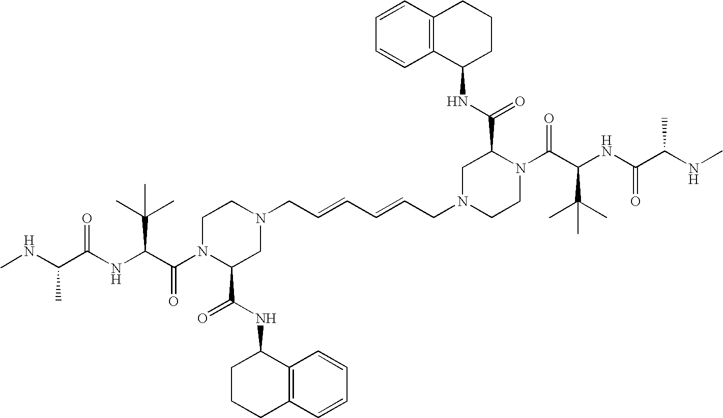

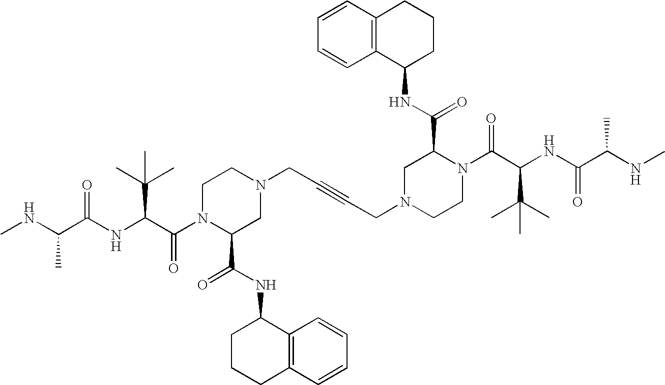

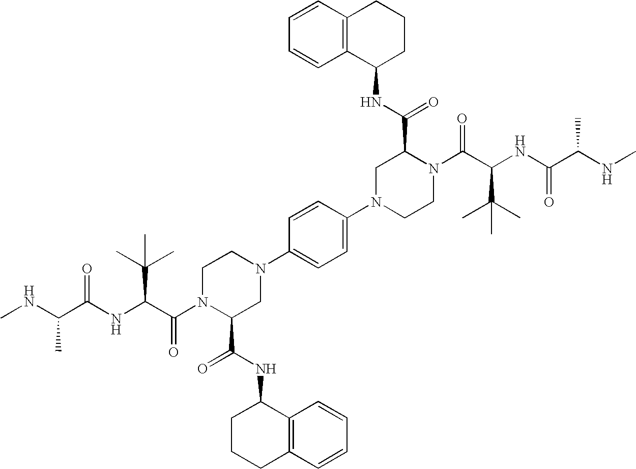

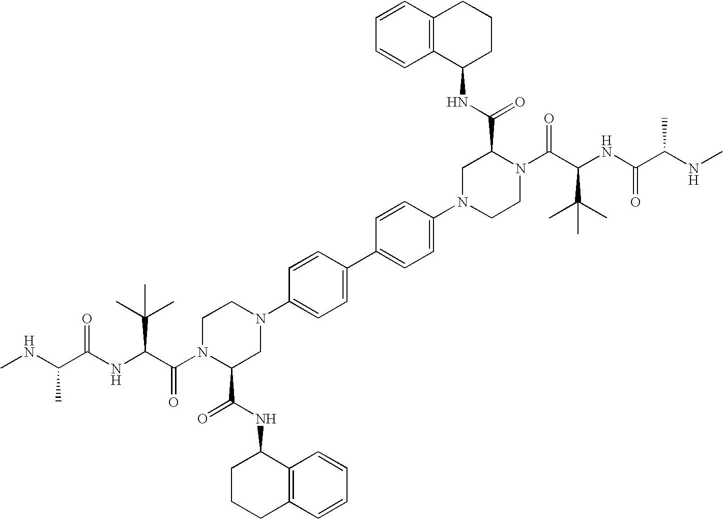

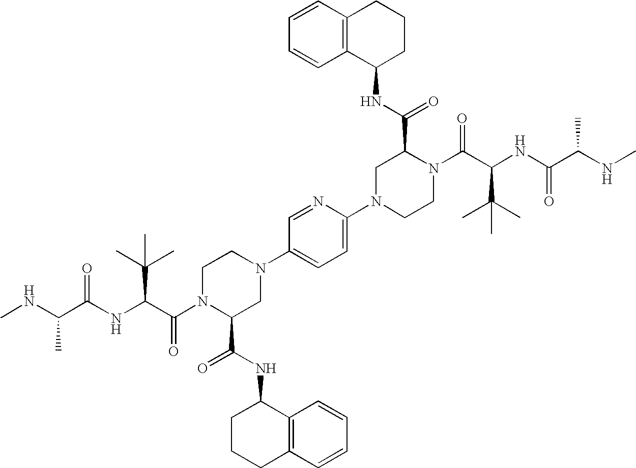

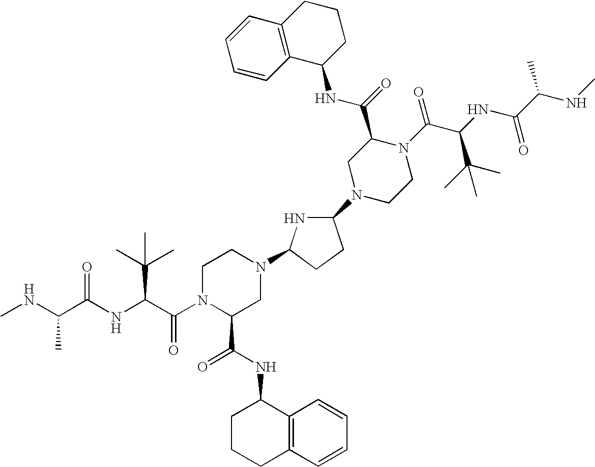

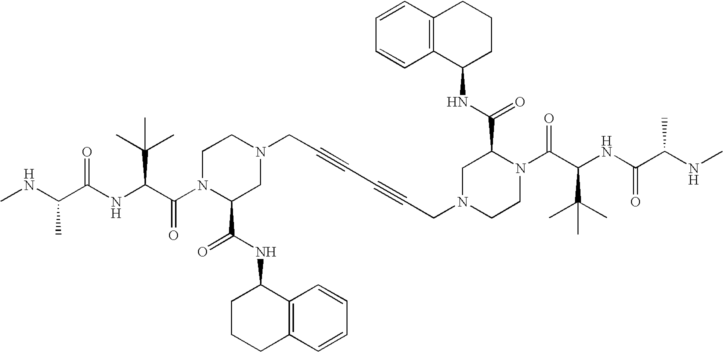

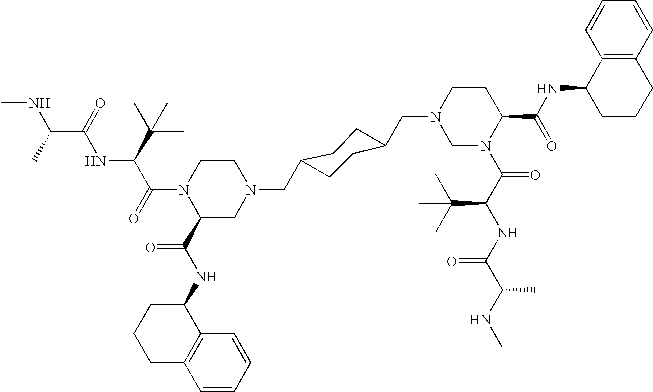

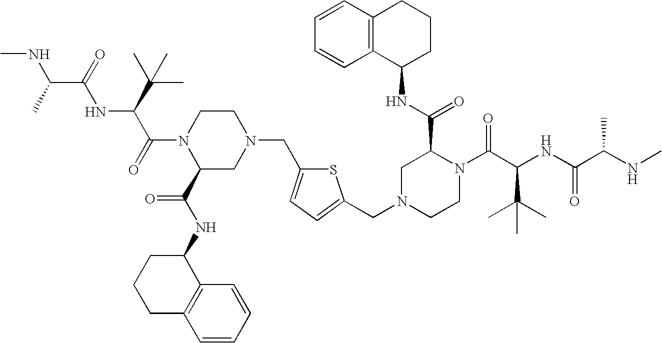

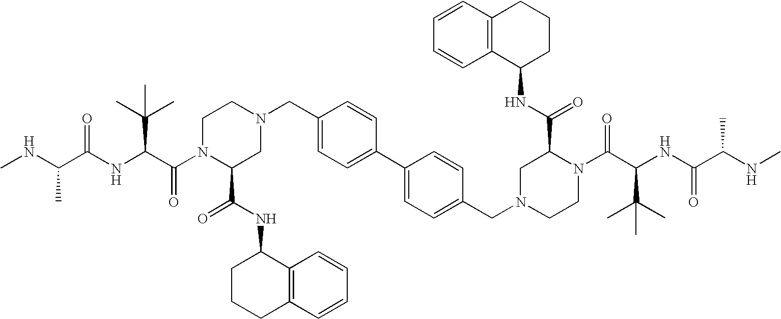

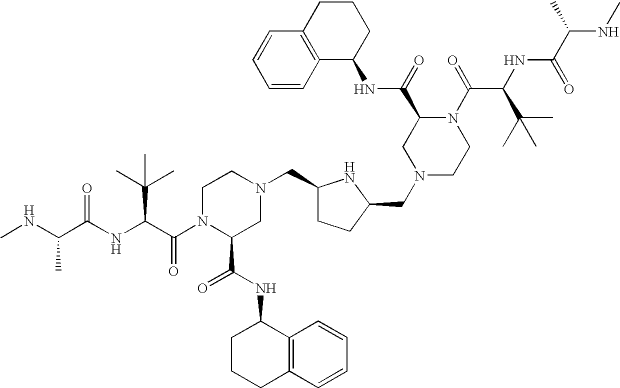

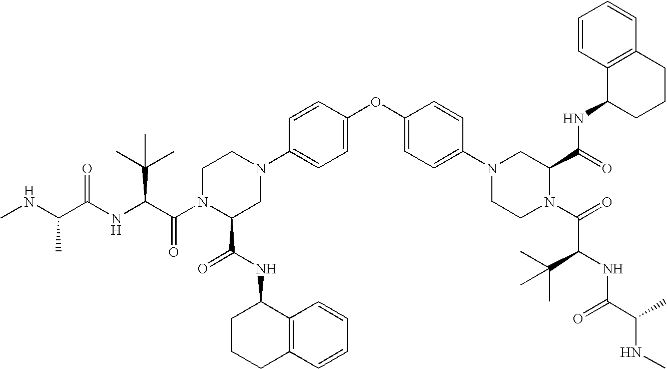

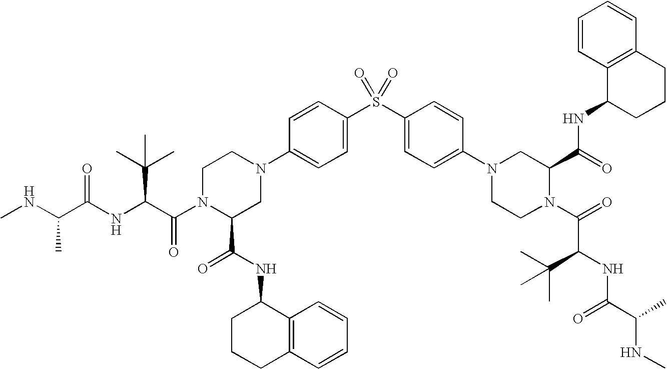

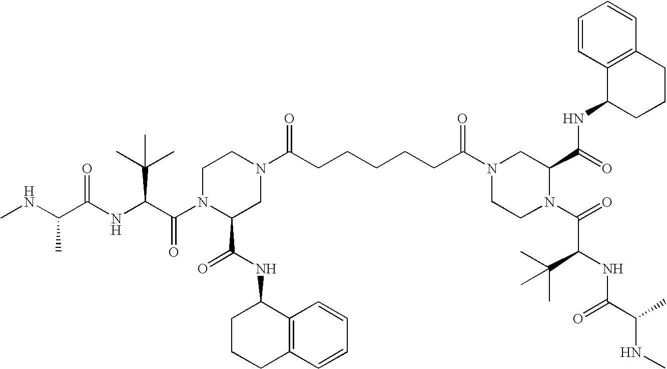

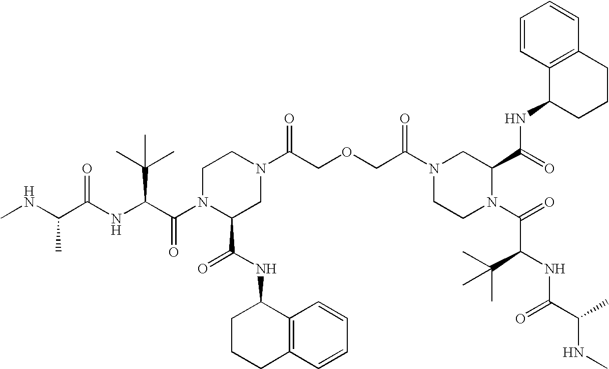

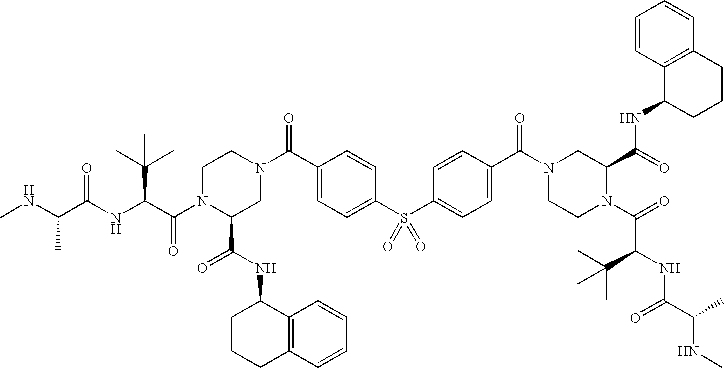

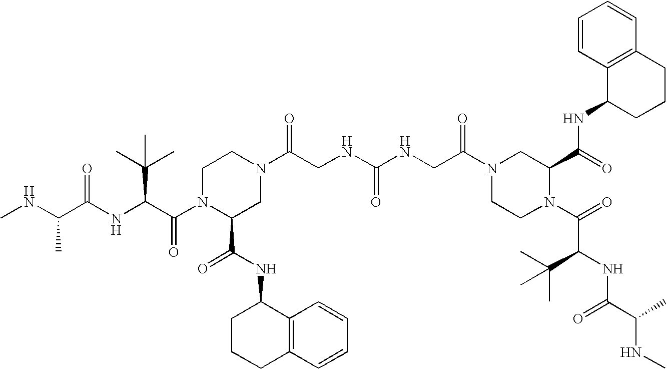

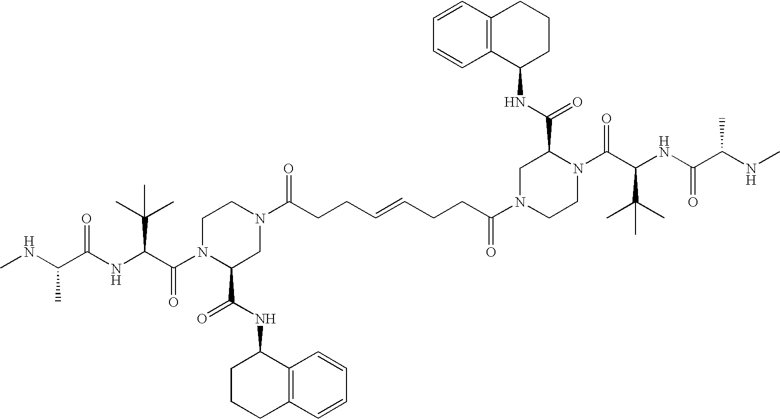

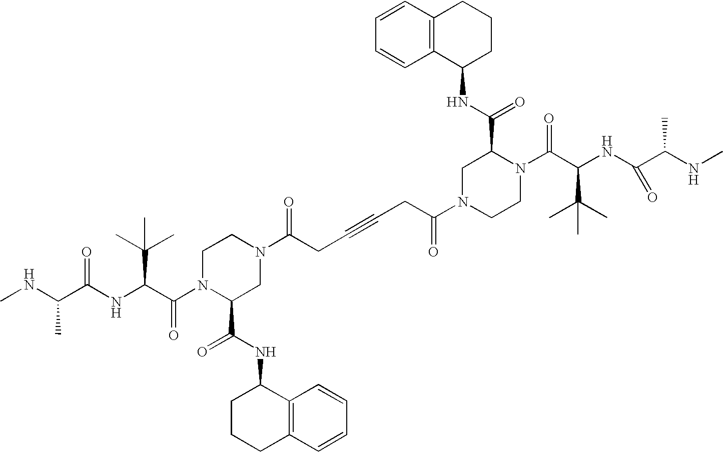

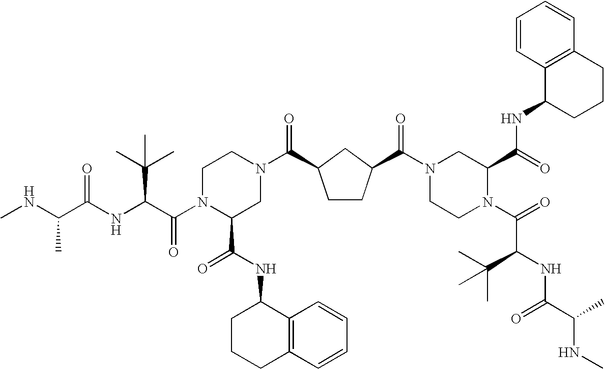

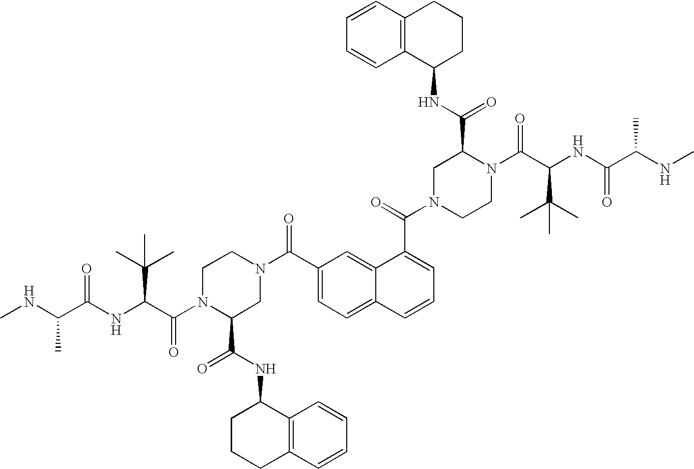

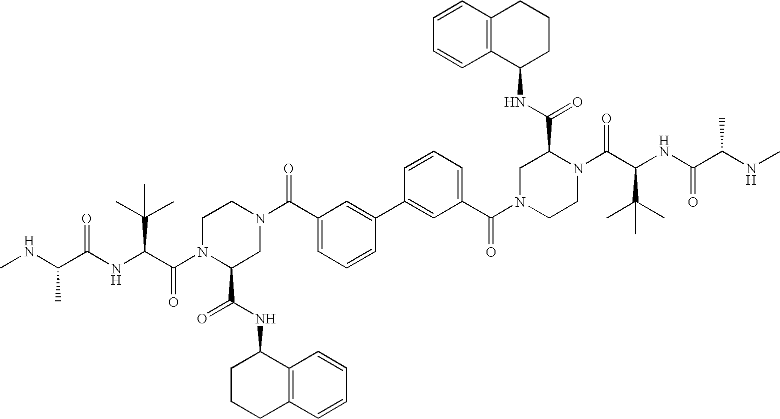

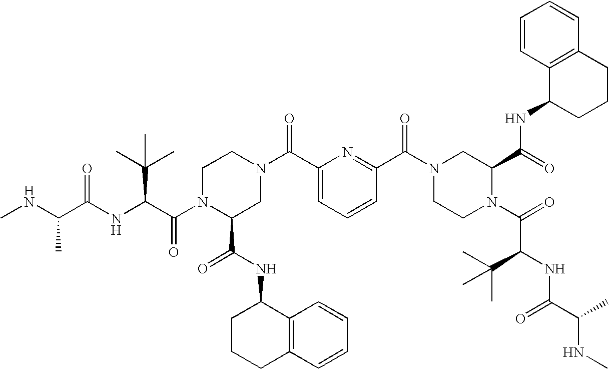

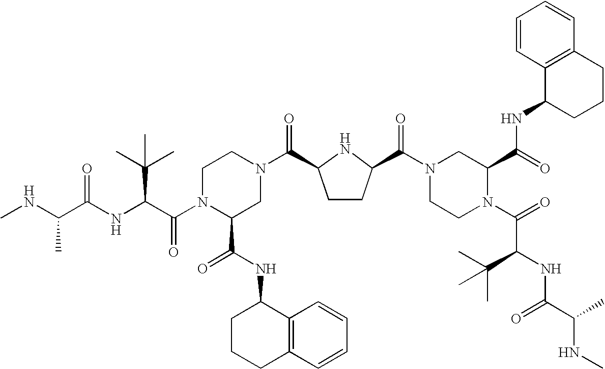

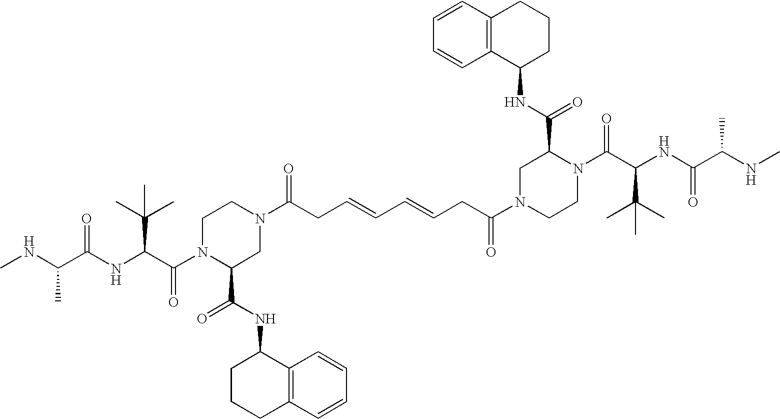

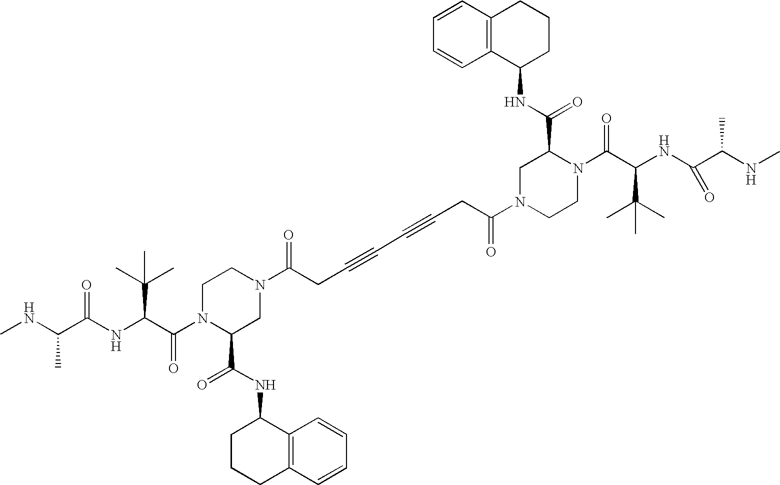

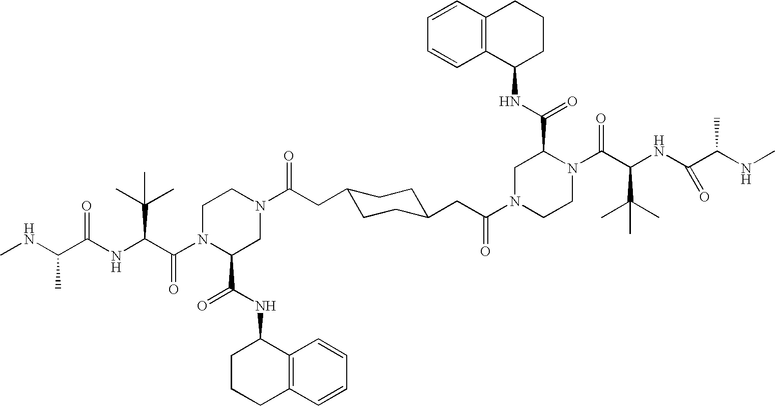

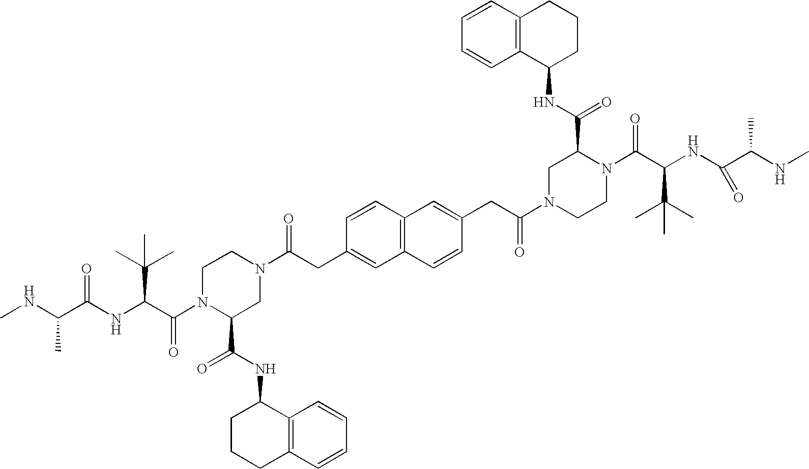

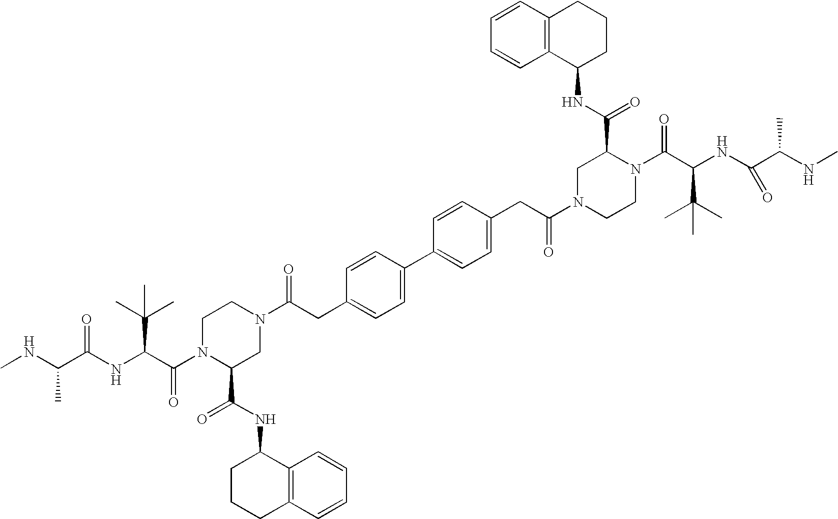

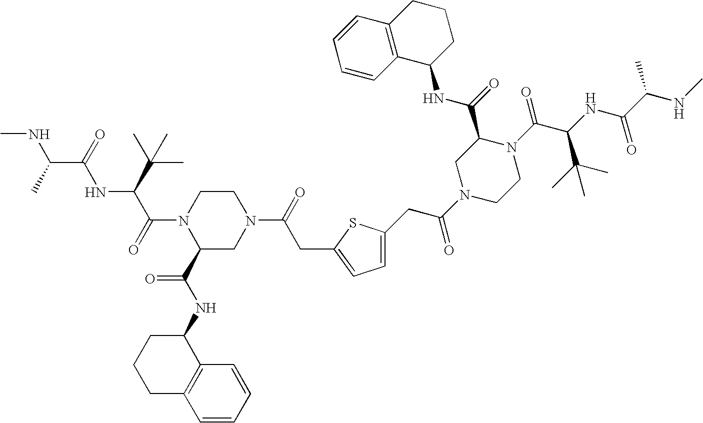

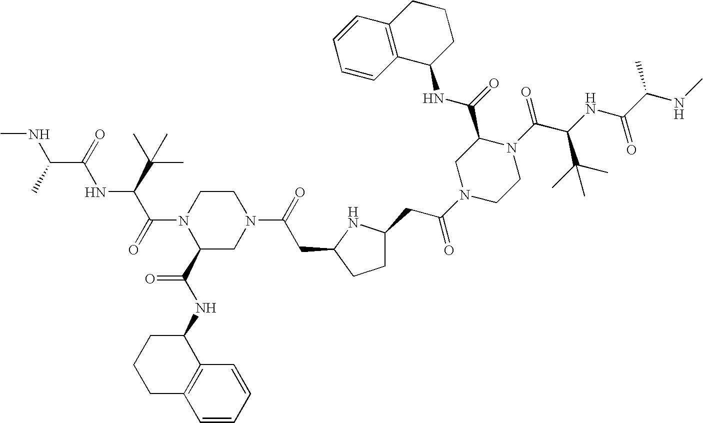

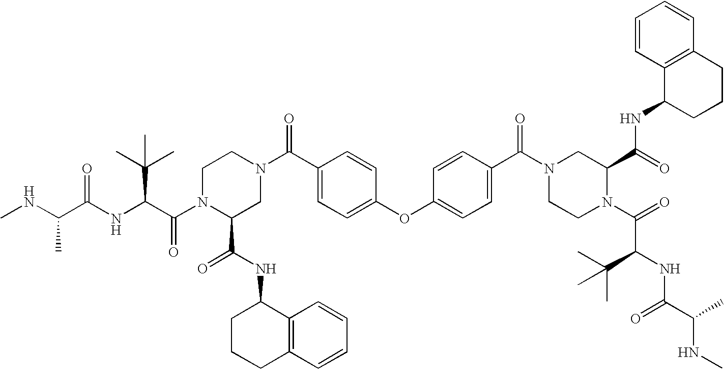

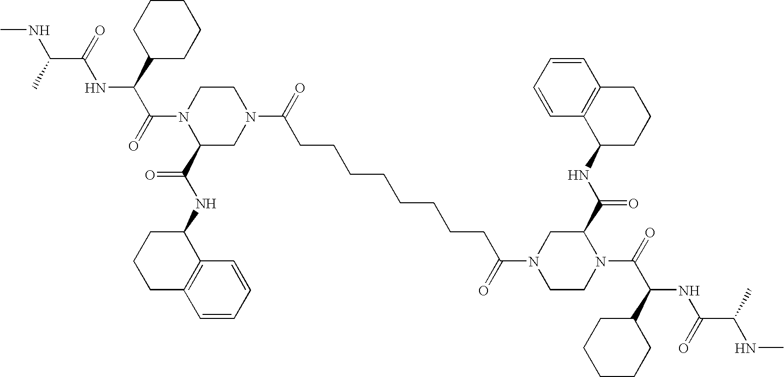

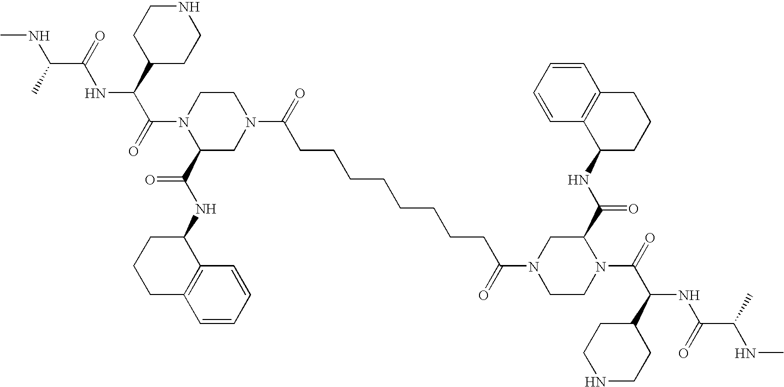

### Assays

### Molecular Constructs for Expression

GST-XIAP BIR3RING: XIAP coding sequence amino acids 246-497 cloned into PGEX2T1 via BamH1 and AVA I. The plasmid was transformed into E. coli DH5α for use in protein expression and purification.

GST-HIAP2 (cIAP-1) BIR 3: HIAP2 coding sequence from amino acids 251-363 cloned into PGex4T3 via BamH1 and XhoI. The plasmid was transformed into E. coli DH5α for use in protein expression and purification.

GST-HIAP1(cIAP-2) BIR 3: HIAP1 coding sequence from amino acids 236-349, cloned into PGex4T3 via BamH1 and XhoI. The plasmid was transformed into E. coli DH5α for use in protein expression and purification.

GST-linker BIR 2 BIR3Ring: XIAP coding sequence from amino acids 93-497 cloned into PGex4T1 via BamH1 and XhoI. Amino acids 93-497 were amplified from full length XIAP in pGex4t3, using the primers: TTAATAGGATCCATCAACGGCTTTTATC and GCTGCATGTGTGTCAGAGG, using standard PCR conditions. The PCR fragment was TA cloned into pCR-2.1 (invitrogen). Linker BIR 2 BIR 3Ring was subcloned into pGex4T1 by BamHI/XhoI digestion. The plasmid was transformed into E. coli DH5α for use in protein expression and purification.

Full-length human XIAP, AEG plasmid number 23. XIAP coding sequence amino acids 1-497 cloned into GST fusion vector, PGEX4T1 via BamH1 and Xho I restriction sites. The plasmid was transformed into E. coli DH5α for use in protein purification.

GST-XIAP linker BIR 2: XIAP linker BIR 2 coding sequence from amino acids 93-497 cloned into pGex4T3 via BamHI and XhoI. The plasmid was transformed into E. coli DH5α for use in protein expression and purification.

### Expression and Purification of Recombinant Proteins

### A. Expression of Recombinant Proteins

Glutathione S-transferase (GST) tagged proteins were expressed in Escherichia coli strains DH5-alpha. For expression of full length XIAP, individual or combinations of XIAP-BIR domains, cIAP-1, cIAP-2 and Livin transformed bacteria were cultured overnight at 37° C. in Luria Broth (LB) medium supplemented with 50 ug/ml of ampicillin. The overnight culture was then diluted 25 fold into fresh LB ampicillin supplemented media and bacteria were grown up to A₆₀₀=0.6 then induced with 1 mM isopropyl-D-1-thiogalactopyranoside for 3 hours. Upon induction, cells were centrifuged at 5000 RPM for 10 minutes and the media was removed. Each pellet obtained from a 1 liter culture received 10 ml of lysis buffer (50 mM Tris-HCl, 200 mM NaCl, 1 mM DTT, 1 mM PMSF, 2 mg/ml of lysosyme, 100 μg/ml)), was incubated at 4° C. with gentle shaking. After 20 minutes of incubation, the cell suspension was placed at −80° C. overnight or until needed.

### B. Purification of Recombinant Proteins

For purification of recombinant proteins, the IPTG-induced cell lysate was thawed vortexed and then disrupted by flash freezing in liquid nitrogen two times with vortexing after each thaw. The cells were disrupted further by passing the extract four times through a Bio-Neb Cell disrupter device (Glas-col) set at 100 psi with Nitrogen gas. The extract was clarified by centrifugation at 4° C. at 15000 RPM in a SS-34 Beckman rotor for 30 minutes. The resulting supernatant was then mixed with 2 ml of glutathione-Sepharose beads (Pharmacia) per 500 ml cell culture (per 1000 ml culture for full length XIAP) for 1 hour at 4° C. Afterwards, the beads were washed 3 times with 1× Tris-Buffered Saline (TBS) to remove unbound proteins. The retained proteins were eluted with 2 washes of 2 ml of 50 mM TRIS pH 8.0 containing 10 mM reduced glutathione. The eluted proteins were pooled and precipitated with 604 g/liter of ammonium sulfate and the resulting pellet re-suspended into an appropriate buffer. As judged by SDS-PAGE the purified proteins were >90% pure. The protein concentration of purified proteins was determined from the Bradford method.

His-tag proteins were expressed in the E. Coli strain in E. coli AD494 cells using a pet28ACPP32 construct. The soluble protein fraction was prepared as described above. For protein purification, the supernatant was purified by affinity chromatography using chelating-Sepharose (Pharmacia) charged with NiSO₄ according to the manufacturer's instructions. Purity of the eluted protein was >90% pure as determined by SDS-PAGE. The protein concentration of purified proteins was determined from the Bradford assay.

### Synthesis of Fluorescent Probe P1

A fluorescent peptide probe, Fmoc-Ala-Val-Pro-Phe-Tyr(t-Bu)-Leu-Pro-Gly(t-Bu)-Gly-OH was prepared using standard Fmoc chemistry on 2-chlorotrityl chloride resin (see Int. J. Pept. Prot. Res. 38:555-561, 1991). Cleavage from the resin was performed using 20% acetic acid in dichloromethane (DCM), which left the side chain still blocked. The C-terminal protected carboxylic acid was coupled to 4′-(aminomethyl)fluorescein (Molecular Probes, A-1351; Eugene, Oreg.) using excess diisopropylcarbodiimide (DIC) in dimethylformamide (DMF) at room temperature and was purified by silica gel chromatography (10% methanol in DCM). The N-terminal Fmoc protecting group was removed using piperidine (20%) in DMF, and purified by silica gel chromatography (20% methanol in DCM, 0.5% HOAc). Finally, the t-butyl side chain protective groups were removed using 95% trifluoroacetic acid containing 2.5% water and 2.5% triisopropyl silane, to provide probe P1 (>95% pure, HPLC).

### Probe P2

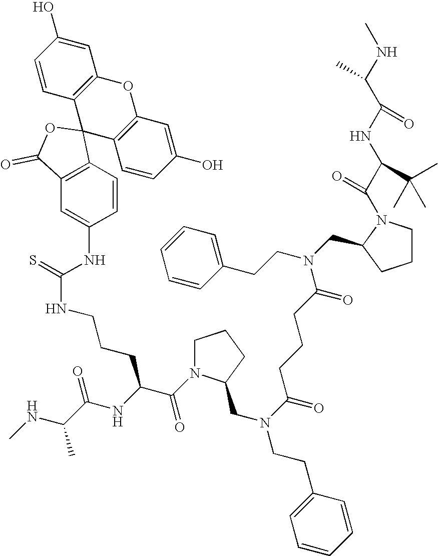

Probe P2 was prepared using methods as described in WO 2007/131,366.

### Binding Assay

### Fluorescence Polarization-Based Competition Assay

For all assays, the fluorescence and fluorescence-polarization was evaluated using a Tecan Polarion instrument with the excitation filter set at 485 nm and the emission filter set at 535 nm. For each assay, the concentration of the target protein was first established by titration of the selected protein in order to produce a linear dose-response signal when incubated alone in the presence of the fluorescent probe P1 or P2. Upon establishing these conditions, the compounds potency (IC₅₀) and selectivity, was assessed in the presence of a fix defined-amount of target protein and fluorescent probe and a 10 point serial dilution of the selected compounds. For each IC₅₀ curve, the assays were run as followed: 25 uL/well of diluted compound in 50 mM MES buffer pH 6.5 was added into a black 96 well plate then 25 ul/well of bovine serum albumin (BSA) at 0.5 mg/ml in 50 mM MES pH 6.5. Auto-fluorescence for each compound was first assessed by performing a reading of the compound/BSA solution alone. Then 25 uL of the fluorescein probe (P1 or P2) diluted into 50 mM MES containing 0.05 mg/ml BSA were added and a reading to detect quenching of fluorescein signal done. Finally 25 uL/well of the target or control protein (GST-BIRs) diluted at the appropriate concentration in 50 mM MES containing 0.05 mg/ml BSA were added and the fluorescence polarization evaluated.

### Determination of IC₅₀ and Inhibitory Constants

For each assay the relative polarization-fluorescence units were plotted against the final concentrations of compound and the IC₅₀ calculated using the Grad pad prism software and/or Cambridge soft. The ki value were derived from the calculated IC₅₀ value as described above and according to the equation described in Nikolovska-Coleska, Z. (2004) Anal Biochem 332, 261-273.

### Fluorescence Polarization Competition Assay

The kᵢ of various compounds in the BIR2-BIR3-ring FP assay, using probe P2, was determined as described above. For example, compound 3 displayed a kᵢ of less than 100 nM.

### Caspase-3 Full Length XIAP, Linker BIR2 or Linker-BIR2-BIR3-RING Derepression Assay

In order to determine the relative activity of the selected compound against XIAP-Bir2, we setup an in vitro assay where caspase-3 was inhibited by GST fusion proteins of XIAP linker-Bir2, XIAP Linker Bir2-Bir3-RING or full-length XIAP. Caspase 3 (0.125 ul) and 12.25-34.25 nM (final concentration) of GST-XIAP fusion protein (GST-Bir2, GST-Bir2Bir3RING or full-length XIAP) were co-incubated with serial dilutions of compound (200 uM-5 pM). Caspase 3 activity was measured by overlaying 25 uL of a 0.4 mM DEVD-AMC solution. Final reaction volume was 100 uL. All dilutions were performed in caspase buffer (50 mM Hepes pH 7.4, 100 mM NaCl, 10% sucrose, 1 mM EDTA, 10 mM DTT, 0.1% CHAPS (Stennicke, H. R., and Salvesen, G. S. (1997). Biochemical characteristics of caspase-3, -6, -7, and -8. J. Biol. Chem. 272, 25719-25723).

The fluorescent AMC released from the caspase-3 hydrolysis of the substrate was measured in a TECAN spectrophotometer at 360 nm excitation and 444 nm emission, after 15 minutes of incubation at room temperature. IC₅₀ values were calculated on a one or two-site competition model using GraphPad v4.0, using the fluorescence values after 15 minutes of incubation plotted against the log 10 concentration of compound.

### Cell-Free Assay

### Caspase De-Repression Assay Using Cellular Extracts (Apoptosome Assay)

100 ug of 293 cell S100 extract and 0.25 uM-2 uM of GST-XIAP fusion protein (XIAP-Bir3RING, XIAP-Bir2Bir3RING, or full-length XIAP) were co-incubated with serial dilutions of compound (40 uM-5 pM). Caspases present in the extracts were activated by adding 1 mM dATP, 0.1 mM ALLN, 133 ug Cytochrome C (final concentrations), and incubating at 37° C. for 25 minutes. All reactions and dilutions used S100 buffer (50 mM Pipes pH 7.0, 50 mM KCl, 0.5 mM EGTA pH 8.0, 2 mM MgCl2 supplemented with 1/1000 dilutions of 2 mg/ml Cytochalisin B, 2 mg/ml Chymostatin, Leupeptin, Pepstatin, Antipain, 0.1M PMSF, 1M DTT). Final reaction volume was 30 ul. Caspase-3 activity was measured by overlaying 30 ul of a 0.4 mM DEVD-AMC solution. Released AMC cleavage was measured in a TECAN spectrophotometer at 360 nm excitation and 444 nm emission, on a kinetic cycle of 1 hour with readings taken every 5 minutes. Caspase activity was calculated as Vₒ of AMC fluorescence/sec. Caspase de-repression by our compounds was compared to fully activated extract and activated extract repressed by the presence of XIAP fusion protein.

### Cell Culture and Cell Death Assays

### A. Cell Culture

SKOV3 (ovarian) and HCT-116 (colon) cancer cells were cultured in McCoy's 5A media supplemented with 10% FBS and 100 units/mL of Penicillin and Streptomycin.

### B. Assays

Cytotoxicity assays were performed on various cell lines including SKOV3 and HCT-116 cells. Cells were seeded in 96 well plates at a respective density of 2000 and 5000 cells per well and incubated at 37° C. in presence of 5% CO₂ for 24 hours. Selected compounds were diluted into the media at various concentrations ranging from 0.01 nM up to 100 nM. Diluted compounds were added onto the SKOV3 cells. HCT-116 cells were co-treated with Trail receptor monoclonal antibody agonist (HGS-ETR¹, 40 ng/mL). After 72 hours, cellular viability was evaluated by MTT conversion. A solution of 3-(4,5-dimethylthiazol-2-yl)-2,5-diphenyltetrazolium bromide (MTT) was added onto cells for a period of 1 to 4 hours. Media was removed and replaced with isopropanol. Conversion of MTT by viable cells was detected by absorbance at 570 nM. The percentage of viability was expressed in percentage of the signal obtained with non treated cells.

Select compounds of the present invention are cytotoxic to SKOV3 cells with EC₅₀ values of 100 nM or less. EC₅₀ values for cytotoxicity of compounds of the present invention to HCT116 cells in the presence of agonist Trail receptor monoclonal antibody is in the range of 50 nM or less.

As seen in Table 7, compounds represented in Table 1 hereinabove potently reduce the viability of SKOV-3 cells. Further compounds of the instant invention reduced the viability of HCT116 cells in the presence of HGS-ETR¹.

TABLE 7

| | | HCT116 + HGS- |
|---|---|---|
| | SKOV3 | ETR1 |
| Compound | EC₅₀ (nM) | EC₅₀ (nM) |
| 1 | A | A |
| 2 | A | A |

A - EC₅₀ less than 50 nM
B - EC⁵⁰ less than 250 nM
C - EC₅₀ more than 1000 nM
D - EC₅₀ more than 1000 nM

Apoptosis Assay: Measurement of Caspase-3 Activity from Cultured Cells

One day, prior to the treatment, 10,000 cells per well were plated in a white tissue culture treated 96 well plate with 100 uL of media. On the day of compound treatment, compounds were diluted with cell culture media to a working stock concentration of 2× and 100 ul of diluted compound were added to each well and the plate was incubated for 5 h at 37° C. in presence of 5% CO₂. Upon incubation, the plate was washed twice with 200 uL of cold TRIS Buffered Saline (TBS) buffer. Cells were lysed with 50 ul of Caspase assay buffer (20 mM Tris-HCl pH 7.4, 0.1% NP-40, 0.1% Chaps, 1 mM DTT, 0.1 mM EDTA, 0.1 mM PMSF, 2 mg/ml Chymostatin, Leupeptin, Pepstatin, Antipapin) then incubated at 4° C. with shaking for 30 minutes. 45 ul of Caspase assay buffer and 5 uL of Ac-DEVD-AMC at 1 mg/ml were added to each well, the plate shaken and incubated for 16 h at 37° C. The amount of release AMC was measured in a TECAN spectrophotometer at with the excitation and emission filter set at 360 nm and 444 nm. The percentage of Caspase-3 activity was expressed in comparison of the signal obtained with the non-treated cells.

### Cellular Biochemistry:

### A. Detection of XIAP and PARP/Caspase-3/Caspase-9

Detection of cell expressed XIAP and PARP were done by western blotting. Cells were plated at 300 000 cells/well in a 60 mm wells (6 wells plate dish). The next day the cells were treated with selected compound at the indicated concentration. 24 hours later cells the trypsinized cells, pelleted by centrifugation at 1800 rpm at 4° C. The resulting pellet was rinsed twice with cold TBS. The final washed pellet of cells was the lysed with 250 uL Lysis buffer (NP-40, glycerol, 1% of a protease inhibitor cocktail (Sigma)), placed at 4° C. for 25 min with gentle shaking. The cells extract was centrifuged at 4° C. for 10 min at 10,000 rpm. Both the supernatant and the pellet were kept for western blotting analysis as described below. From the supernatant, the protein content was evaluated and about 50 ug of protein was fractionated onto a 10% SDS-PAGE. Pellets were washed with the lysis buffer and re-suspend into 50 ul of Lamelli buffer 1×, boiled and fractionated on SDS-PAGE. Upon electrophoresis each gel was electro-transferred onto a nitrocellulose membrane at 0.6 A for 2 hours. Membrane non-specific sites were blocked for 1 hours with 5% Skim milk in TBST (TBS containing 0.1% (v/v) Tween-20) at RT. For protein immuno-detection, membranes were incubated overnight with primary antibodies raised against XIAP clone 48 obtained from Becton-Dickison) or PARP: obtained from Cell signal or caspase-3 or caspase-9 primary antibodies were incubated at 4° C. with shaking at dilutions as follows:

| | |
|---|---|
| XIAP clone 80 (Becton-Dickinson) | 1/2500 |
| PARP (Cell Signal) | 1/2500 |
| Caspase 3 (Sigma) | 1/1500 |
| Caspase 9 (Upstate) | 1/1000 |

Upon overnight incubation, the membranes received three washes of 15 min in TBST then were incubated for 1 hour at room temperature in the presence of a secondary antibody coupled with HRP-enzyme (Chemicon) and diluted at 1/5 000. Upon incubation each membrane were washed three times with TBST and the immunoreactive bands were detected by addition of a luminescent substrate (ECL kit Amersham) and capture of signal on a X-RAY film for various time of exposure.

### Hollow Fiber Model

Hollow fiber in vivo model were used to demonstrate in vivo efficacy of selected compounds against selected cell lines as single agent therapy or in combination with selected cytotoxic agents. At day 1, selected cell lines were cultured and the fiber filled at a cell density of about 40,000 cells/fiber. At the day of operation (day 4), three fibers are implanted sub-cutaneous into 28-35 Nu/Nu CD-1 male mice. On day 5, mice start to receive daily injection via sub-cutaneous route of control vehicle or vehicle containing the selected compound at the appropriate concentration and/or injection of cytotoxic agent via intra-peritoneal route. Upon 3-7 days of consecutive drug treatments, the animals are sacrificed, each fiber is removed and the metabolic viability of the remaining cells determined by MTT assay. Efficacy of the compound is defined as the difference between the vehicle-treated animal and the animal treated with the compound alone or the compound given in combination of the cytotoxic agent.

### SKOV-3 Human Ovarian Cancer Cell Line Xenograft Study

Female CD-1 nude mice (approximately 20-25 g) were subcutaneously injected 5×10⁶ SKOV-3 human ovarian tumor cells in 50% matrigel subcutaneously in the right flank. On day 55, when tumors were approximately 100 mm³, treatment was initiated with compound 3 on a 5 on/2 off treatment schedule for the duration of the experiment. Tumor size was measured with digital calipers and calculated as V=(a×b²)/2, wherein, a is the longest dimension and b is the width.

### MDA-MB-231 Human Mammary Cancer Cell Line Xenograft Study

Female CD-1 nude mice (approximately 20-25 g) are subcutaneously injected 1×106 MDA-MB-231 human mammary tumor cells in the right flank. On day 71, when tumors are approximately 90 mm³, treatment is initiated with compounds of formula I. Tumor size is measured with digital calipers and calculated as V=(a×b²)/2, wherein, a is the longest dimension and b is the width.

### Pharmacokinetic Studies

Selected compounds are dissolved into saline or D5W and given at various doses using different route of administration, including intravenous bolus, intravenous infusion, oral and subcutaneous injection.

### Other Embodiments

From the foregoing description, it will be apparent to one of ordinary skill in the art that variations and modifications may be made to the invention described herein to adapt it to various usages and conditions. Such embodiments are also within the scope of the present invention.

All publications mentioned in this specification are hereby incorporated by reference.

### REFERENCES

1. Deveraux Q L, Reed J C. IAP family proteins—suppressors of apoptosis. Genes Dev 1999; 13(3):239-52.
2. Riedl S J, Shi Y. Molecular mechanisms of caspase regulation during apoptosis. Nat Rev Mol Cell Biol 2004; 5(11):897-907.
3. Lacasse E C, Kandimalla E R, Winocour P, Sullivan T, Agrawal S, Gillard J W, et al. Application of XIAP antisense to cancer and other proliferative disorders: development of AEG35156/GEM640. Ann N Y Acad Sci 2005; 1058:215-34.
4. Tamm I, Kornblau S M, Segall H, Krajewski S, Welsh K, Kitada S, et al. Expression and prognostic significance of IAP-family genes in human cancers and myeloid leukemias. Clin Cancer Res 2000; 6(5):1796-803.
5. Ramp U, Krieg T, Caliskan E, Mahotka C, Ebert T, Willers R, et al. XIAP expression is an independent prognostic marker in clear-cell renal carcinomas1. Hum Pathol 2004; 35(8):1022-8.
6. Tamm I, Richter S, Oltersdorf D, Creutzig U, Harbott J, Scholz F, et al. High expression levels of x-linked inhibitor of apoptosis protein and survivin correlate with poor overall survival in childhood de novo acute myeloid leukemia. Clin Cancer Res 2004; 10(11):3737-44.
7. Nakagawa Y, Abe S, Kurata M, Hasegawa M, Yamamoto K, Inoue M, et al. IAP family protein expression correlates with poor outcome of multiple myeloma patients in association with chemotherapy-induced overexpression of multidrug resistance genes. Am J Hematol 2006; 81(11):824-31.
8. Mizutani Y, Nakanishi H, Li Y N, Matsubara H, Yamamoto K, Sato N, et al. Overexpression of XIAP expression in renal cell carcinoma predicts a worse prognosis. Int J Oncol 2007; 30(4):919-25.
9. Sasaki H, Sheng Y, Kotsuji F, Tsang B K. Down-regulation of X-linked inhibitor of apoptosis protein induces apoptosis in chemoresistant human ovarian cancer cells. Cancer Res 2000; 60(20):5659-66.
10. Carter B Z, Milella M, Tsao T, McQueen T, Schober W D, Hu W, et al. Regulation and targeting of antiapoptotic XIAP in acute myeloid leukemia. Leukemia 2003; 17(11):2081-9.
11. Amantana A, London C A, Iversen P L, Devi G R. X-linked inhibitor of apoptosis protein inhibition induces apoptosis and enhances chemotherapy sensitivity in human prostate cancer cells. Mol Cancer Ther 2004; 3(6):699-707.
12. Cao C, Mu Y, Hallahan D E, Lu B. XIAP and survivin as therapeutic targets for radiation sensitization in preclinical models of lung cancer. Oncogene 2004.
13. Lima R T, Martins L M, Guimaraes J E, Sambade C, Vasconcelos M H. Specific downregulation of bcl-2 and xIAP by RNAi enhances the effects of chemotherapeutic agents in MCF-7 human breast cancer cells. Cancer Gene Ther 2004; 11(5):309-16.
14. Yamaguchi Y, Shiraki K, Fuke H, Inoue T, Miyashita K, Yamanaka Y, et al. Targeting of X-linked inhibitor of apoptosis protein or survivin by short interfering RNAs sensitize hepatoma cells to TNF-related apoptosis-inducing ligand- and chemotherapeutic agent-induced cell death. Oncol Rep 2005; 14(5):1311-6.
15. Kashkar H, Seeger J M, Hombach A, Deggerich A, Yazdanpanah B, Utermohlen O, et al. XIAP targeting sensitizes Hodgkin lymphoma cells for cytolytic T-cell attack. Blood 2006; 108(10):3434-40.
16. Ohnishi K, Scuric Z, Schiestl R H, Okamoto N, Takahashi A, Ohnishi T. siRNA targeting NBS1 or XIAP increases radiation sensitivity of human cancer cells independent of TP53 status. Radiat Res 2006; 166(3):454-62.
17. Shrikhande S V, Kleeff J, Kayed H, Keleg S, Reiser C, Giese T, et al. Silencing of X-linked inhibitor of apoptosis (XIAP) decreases gemcitabine resistance of pancreatic cancer cells. Anticancer Res 2006; 26(5A):3265-73.
18. LaCasse E C, Cherton-Horvat G G, Hewitt K E, Jerome L J, Morris S J, Kandimalla E R, et al. Preclinical characterization of AEG35156/GEM 640, a second-generation antisense oligonucleotide targeting X-linked inhibitor of apoptosis. Clin Cancer Res 2006; 12(17):5231-41.
19. Naumann U, Bahr O, Wolburg H, Altenberend S, Wick W, Liston P, et al. Adenoviral expression of XIAP antisense RNA induces apoptosis in glioma cells and suppresses the growth of xenografts in nude mice. Gene Ther 2007; 14(2):147-61.
20. Vogler M, Durr K, Jovanovic M, Debatin K M, Fulda S. Regulation of TRAIL-induced apoptosis by XIAP in pancreatic carcinoma cells. Oncogene 2007; 26(2):248-57.
21. Zhang S, Ding F, Luo A, Chen A, Yu Z, Ren S, et al. XIAP is Highly Expressed in Esophageal Cancer and its Downregulation by RNAi Sensitizes Esophageal Carcinoma Cell Lines to Chemotherapeutics. Cancer Biol Ther 2007; 6(6).
22. Lopes R B, Gangeswaran R, McNeish I A, Wang Y, Lemoine N R. Expression of the IAP protein family is dysregulated in pancreatic cancer cells and is important for resistance to chemotherapy. Int J Cancer 2007; 120(11):2344-2352.
23. Pender, M. P. 1998. Genetically determined failure of activation-induced apoptosis of autoreactive T cells as a cause of multiple sclerosis. Lancet 351: 978-981.
24. Leaman, D. W., M. Chawla-Sarkar, K. Vyas, M. Reheman, K. Tamai, S. Toji, and E. C. Borden. 2002. Identification of X-linked inhibitor of apoptosis-associated factor-1 as an interferon-stimulated gene that augments TRAIL Apo2L-induced apoptosis. J. Biol. Chem. 277: 28504-28511.
25. Sharief, M. K., and R. Hentges. 1991. Association between tumor necrosis factor- and disease progression in patients with multiple sclerosis. N. Engl. J. Med. 325: 467-472.
26. Sharief, M. K., M. A. Noori, and Y. Zoukos. 2002. Reduced expression of the inhibitor of apoptosis proteins in T cells from patients with multiple sclerosis following interferon-therapy. J. Neuroimmunol. 129: 224-231.
27. Sharief, M. K., and Y. K. Semra. 2001. Upregulation of the inhibitor of apoptosis proteins in activated T lymphocytes from patients with multiple sclerosis. J. Neuroimmunol. 119: 350-357.
28. Sharief, M. K., and Y. K. Semra. 2002. Down-regulation of survivin expression in T lymphocytes after interferon-1a treatment in patients with multiple sclerosis. Arch. Neurol. 59: 1115-1121
29. Perlman, H, Pagliari, L. J., Liu, H., Koch, A. E., Haines, G. K, and Pope, R. M. 2001. Rheumatoid arthritis synovial macrophages express the Fas-associated death domain-like interleukin 1b-converting enzyme-inhibitor protein and are refractory to Fas-mediated apoptosis. Arthritis and Rheumatism, 44: 21-30.
30. Raj, D, Brash, D. E. and Grossman D. 2008. Keratinocyte apoptosis in epidermal development and disease. Journal of Investigational Dermatology 126: 243-257
31. Simone P. Zehntner, Lyne Bourbonnie're, Craig S. Moore, Stephen J. Morris, Danielle Methot, Martine St. Jean, Eric Lacasse, Andrea L. O. Hebb, George S. Robertson, Jon Durkin, John W. Gillard, and Trevor Owens, X-linked Inhibitor of Apoptosis Regulates T Cell Effector Function, The Journal of Immunology, 179, (11), 7553-7560.

1. A compound represented by Formula 1: 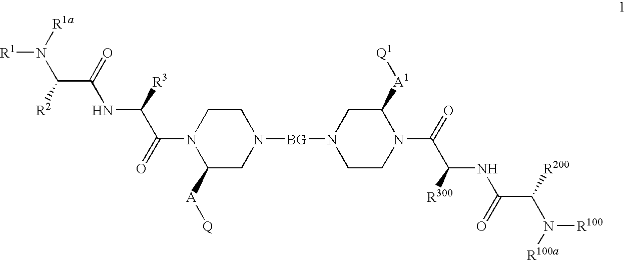
or a salt thereof,
wherein
n is 0 or 1;
m is 0, 1 or 2;
Y is NR⁸, O or S;
BG is:
1) —X-L-X¹—; or
2) L;
X and X¹ are independently: 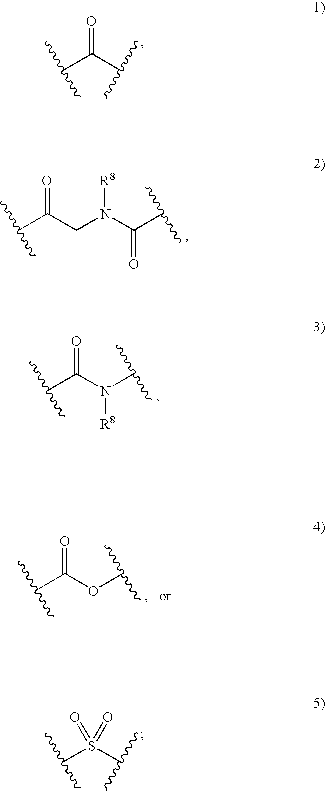
L is:
1) —C₁-C₂₀ alkyl-,
2) —C₂-C₆ alkenyl-,
3) —C₂-C₄ alkynyl-,
4) —C₃-C₇ cycloalkyl-,
5) -aryl-,
6) -biphenyl-,
7) -heteroaryl-,
8) -heterocyclyl-,
9) —C₁-C₆ alkyl-(C₂-C₆ alkenyl)-C₁-C₆ alkyl-,
10) —C₁-C₆ alkyl-(C₂-C₄ alkynyl)-C₁-C₆ alkyl-
11) —C₁-C₆ alkyl-(C₃-C₇ cycloalkyl)-C₁-C₆ alkyl-,
12) —C₁-C₆ alkyl-aryl-C₁-C₆ alkyl-,
13) —C₁-C₆ alkyl-biphenyl-C₁-C₆ alkyl-,
14) —C₁-C₆ alkyl-heteroaryl-C₁-C₆ alkyl-,
15) —C₁-C₆ alkyl-heterocycyl-C₁-C₆ alkyl-,
16) —C₁-C₆ alkyl-Y—C₁-C₆ alkyl-,
17) -aryl-Y-aryl-,
18) —C₁-C₆ alkyl-Z—C₁-C₆ alkyl-, or
19) -aryl-Z-aryl-;
wherein the alkyl, alkenyl, alkynyl and cycloalkyl are optionally substituted with one or more R⁶ substituents, and the aryl, biphenyl, heteroaryl, and heterocyclyl are optionally substituted with one or more R¹⁰ substituents;
Z is:
1) —C(O)—,
2) —S(O)₂—,
3) —N(R⁸)C(O)—,
4) —C(O)N(R⁸)—,
5) —S(O)₂N(R⁸)—,
6) —N(R⁸)—C(O)—N(R⁸)—,
7) —N(R⁸)—C(O)C(O)—N(R⁸)—,
8) —N(R⁸)—C(O)—C₁-C₁₂-alkyl-C(O)—N(R⁸)—,
9) —N(R⁸)—C(O)-aryl-C(O)—N(R⁸)—,
10) —N(R⁸)—C(O)-aryl-O-aryl-C(O)—N(R⁸)—,
11) —N(R⁸)—C(O)-heteroaryl-C(O)—N(R⁸)—,
12) —N(R⁸)—C(O)-biphenyl-C(O)—N(R⁸)—,
13) —N(R⁸)—S(O)₂—C₁-C₁₂-alkyl-S(O)₂—N(R⁸)—,
14) —N(R⁸)—S(O)₂-aryl-S(O)₂—N(R⁸)—,
15) —N(R⁸)—S(O)₂-heteroaryl-S(O)₂—N(R⁸)—,
16) —N(R⁸)—S(O)₂-biphenyl-S(O)₂—N(R⁸)—,
17) —N(R⁸)—C₁-C₁₂-alkyl-N(R⁸)—,
18) —N(R⁸)-aryl-N(R⁸)—,
19) —N(R⁸)-heteroaryl-N(R⁸)—, or
20) —N(R⁸)-biphenyl-N(R⁸)—;
wherein the alkyl is optionally substituted with one or more R⁶ substituents, and the aryl, the heteroaryl, the biphenyl and the heterocyclyl are optionally substituted with one or more R¹⁰ substituents;
Q and Q¹ are independently:
1) —NR⁴R⁵,
2) —OR¹¹,
3) —S(O)ₘR¹¹; or
Q and Q¹ are independently:
1) aryl, or
2) heteroaryl;
wherein the aryl and the heteroaryl are optionally substituted with one or more R¹⁰ substituents;
A and A¹ are independently:
1) —CH₂—,
2) —CH₂CH₂—,
3) —CH(C₁-C₆ alkyl)-,
4) —CH(C₃-C₇ cycloalkyl)-,
5) —C₃-C₇ cycloalkyl-,
6) —CH(C₁-C₆ alkyl-C₃-C₇ cycloalkyl)-,
7) —C(O)—, or
wherein the alkyl and cycloalkyl are optionally substituted with one or more R⁶ substituents;
R¹, R^{1a}, R¹⁰⁰ and R^{100a} are independently:
1) H, or
2) —C₁-C₆ alkyl optionally substituted with one or more R⁶ substituents;
R² and R²⁰⁰ are independently:
1) H, or
2) C₁-C₆ alkyl optionally substituted with one or more R⁶ substituents;
R³ and R³⁰⁰ are independently:
1) H,
2) C₁-C₆ alkyl,
3) C₃-C₇ cycloalkyl,
4) C₃-C₇ cycloalkenyl,
5) aryl,
6) biphenyl,
7) heteroaryl,
8) heterocyclyl,
9) heterobicyclyl,
wherein the alkyl, cycloalkyl and cycloalkenyl are optionally substituted with one or more R⁶ substituents; and wherein the aryl, biphenyl, heteroaryl, heterocyclyl and heterobicyclyl are optionally substituted with one or more R¹⁰ substituents.
R⁴ and R⁵ are each independently:
1) H,
2) haloalkyl,
3) C₁-C₆ alkyl,
4) C₂-C₆ alkenyl,
5) C₂-C₄ alkynyl,
6) C₃-C₇ cycloalkyl,
7) C₃-C₇ cycloalkenyl,
8) aryl,
9) biphenyl,
10) heteroaryl,
11) heterocyclyl,
12) heterobicyclyl,
13) aryl-heteroaryl,
14) heteroaryl-aryl,
15) heterocyclyl-aryl,
16) —C(O)Oₙ—R¹¹,
17) —S(O)₂—R¹¹, or
18) —C(═Y)NR⁸R⁹,
wherein the alkyl, the alkenyl, the alkynyl, the cycloalkyl and the cycloalkenyl are optionally substituted with one or more R⁶ substituents, and the aryl, the biphenyl, the heteroaryl, the heterocyclyl and the heterobicyclyl are optionally substituted with one or more R¹⁰ substituents
R⁶ is:
1) halogen,
2) NO₂,
3) CN,
4) haloalkyl,
5) C₁-C₆ alkyl,
6) C₂-C₆ alkenyl,
7) C₂-C₄ alkynyl,
8) C₃-C₇ cycloalkyl,
9) C₃-C₇ cycloalkenyl,
10) aryl,
11) heteroaryl,
12) heterocyclyl,
13) heterobicyclyl,
14) —OR⁷,
15) —S(O)ₙR⁷,
16) —NR⁸R⁹,
17) —NR⁸S(O)₂R¹¹,
18) —C(O)OₙR⁷,
19) —C(O)NR⁸R⁹,
20) —S(O)₂NR⁸R⁹
21) —OC(O)R⁷,
22) —OC(O)Y—R¹¹,
23) —SC(O)R⁷, or
24) —NC(Y)NR⁸R⁹;
wherein the aryl, heteroaryl, heterocyclyl and heterobicyclyl is optionally substituted with one or more R¹⁰ substituents
R⁷ is:
1) H,
2) haloalkyl,
3) C₁-C₆ alkyl,
4) C₂-C₆ alkenyl,
5) C₂-C₄ alkynyl,
6) C₃-C₇ cycloalkyl,
7) C₃-C₇ cycloalkenyl,
8) aryl,
9) biphenyl,
10) heteroaryl,
11) heterocyclyl,
12) heterobicyclyl,
13) —C₁-C₆ alkyl-C₂-C₄ alkenyl, or
14) —C₁-C₆ alkyl-C₂-C₄ alkynyl,
wherein the alkyl, alkenyl, alkynyl, cycloalkyl and cycloalkenyl are optionally substituted with one or more R⁶ substituents; and wherein the aryl, biphenyl, heteroaryl, heterocyclyl and heterobicyclyl are optionally substituted with one or more R¹⁰ substituents;
R⁸ and R⁹ are each independently:
1) H,
2) -haloalkyl,
3) —C₁-C₆ alkyl,
4) —C₂-C₆ alkenyl,
5) —C₂-C₄ alkynyl,
6) —C₃-C₇ cycloalkyl,
7) —C₃-C₇ cycloalkenyl,
8) aryl,
9) biphenyl,
10) heteroaryl,
11) heterocyclyl,
12) heterobicyclyl,
wherein the alkyl, alkenyl, alkynyl, cycloalkyl and cycloalkenyl are optionally substituted with one or more R⁶ substituents; and wherein the aryl, biphenyl, heteroaryl, heterocyclyl and heterobicyclyl are optionally substituted with one or more R¹⁰ substituents;
or R⁸ and R⁹ together with the nitrogen atom to which they are bonded form a five, six or seven membered heterocyclic ring optionally substituted with one or more R⁶ substituents;
R¹⁰ is:
1) halogen,
2) NO₂,
3) CN,
4) —B(OR¹³)(OR¹⁴),
5) —C₁-C₆ alkyl,
6) —C₂-C₆ alkenyl,
7) —C₂-C₄ alkynyl,
8) —C₃-C₇ cycloalkyl,
9) —C₃-C₇ cycloalkenyl,
10) haloalkyl,
11) —OR⁷,
12) —NR⁸R⁹,
13) —SR⁷,
14) —COR⁷,
15) —C(O)OR⁷,
16) —S(O)ₘR⁷,
17) —CONR⁸R⁹,
18) —S(O)₂NR⁸R⁹,
19) aryl,
20) biphenyl,
21) heteroaryl,
22) heterocyclyl, or
23) heterobicyclyl,
wherein the alkyl, alkenyl, alkynyl, cycloalkyl and cycloalkenyl are optionally substituted with one or more R⁶ substituents;
R¹¹ is:
1) haloalkyl,
2) C₁-C₆ alkyl,
3) C₂-C₆ alkenyl,
4) C₂-C₄ alkynyl,
5) C₃-C₇ cycloalkyl,
6) C₃-C₇ cycloalkenyl,
7) aryl,
8) biphenyl,
9) heteroaryl,
10) heterocyclyl, or
11) heterobicyclyl,
wherein the alkyl, alkenyl, alkynyl, cycloalkyl and cycloalkenyl are optionally substituted with one or more R⁶ substituents; and wherein the aryl, biphenyl, heteroaryl, heterocyclyl and heterobicyclyl are optionally substituted with one or more R¹⁰ substituents;
R¹³ and R¹⁴ are H or C₁-C₆ alkyl, or R¹³ and R¹⁴ are joined to form a 5-6 membered alkyl containing boronate ring system.

2. (canceled)

3. The compound according to claim 1, in which A and A¹ are both C═O.

4. The compound according to claim 1, in which Q and Q¹ are both NR⁴R⁵, wherein R⁴ is H and R⁵ is aryl or C₁-C₆ alkyl, the alkyl being optionally substituted with one or more R⁶ substituents.

5. The compound according to claim 1, in which both Q and Q¹ are:

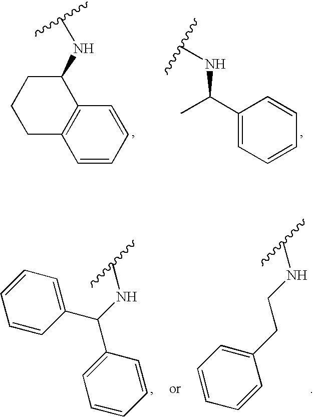

6. The compound, according to claim 1 in which BG is —X-L-X¹—, wherein X and X¹ are independently: 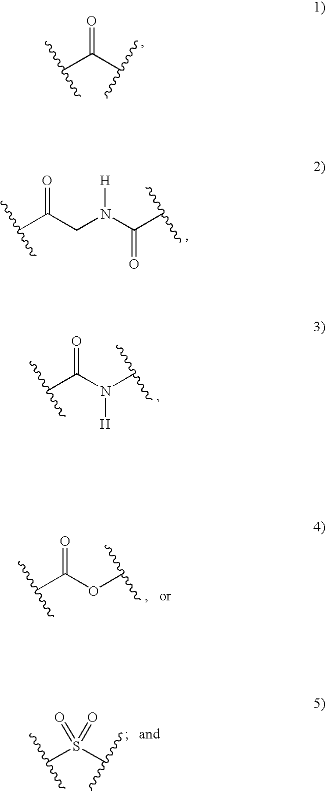
L is:
1) —C₁-C₂₀ alkyl-,
2) —C₃-C₇ cycloalkyl-,
3) -aryl-,
4) -biphenyl-,
5) -heteroaryl-, or
6) -aryl-Y-aryl-,
wherein the alkyl and cycloalkyl are optionally substituted with one or more R⁶ substituents, and the aryl, biphenyl, and heteroaryl are optionally substituted with one or more R¹⁰ substituents.

7. The compound according to claim 1, in which BG is:

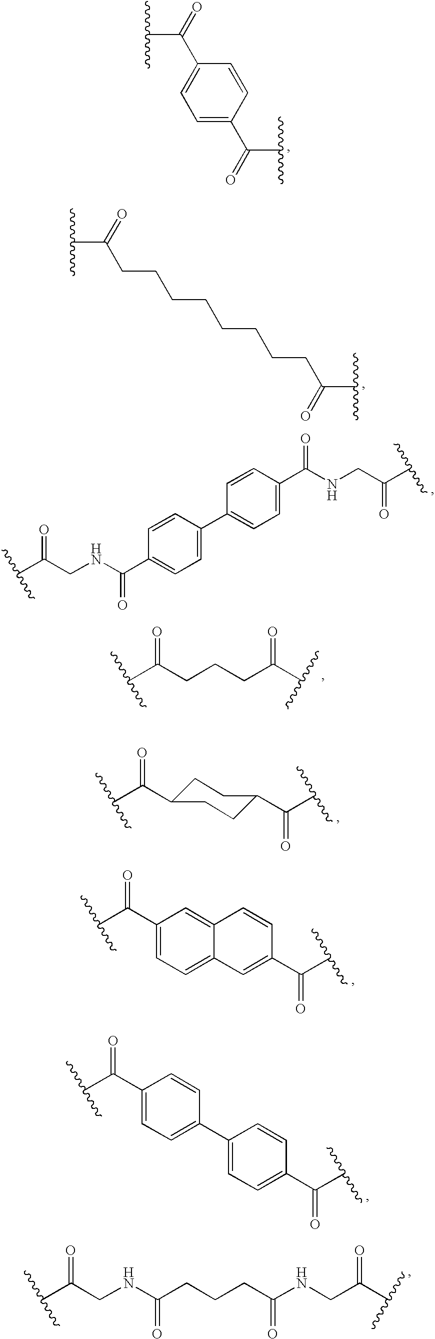

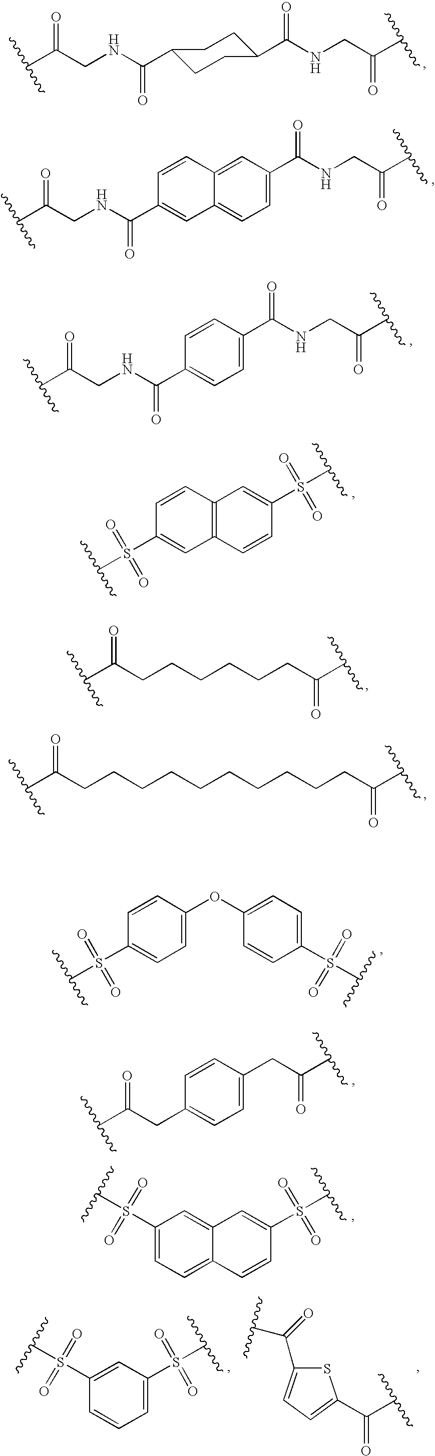

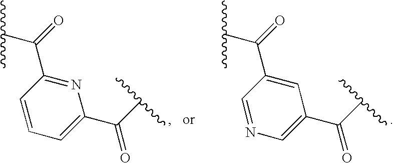

8. The compound, according to claim 1, in which R^{1a} and R^{100a} are both H, and R¹ and R¹⁰⁰ are both —CH₃.

9. The compound according to claim 1, in which R² and R²⁰⁰ are both —CH₃.

10. The compound according to claim 1, in which R³ and R³⁰⁰ are both —C(CH₃)₃.

11. A compound according to claim 1, wherein the compound is:

| Com- | |
|---|---|
| pound | |
| No. | Structure |
| 1 | 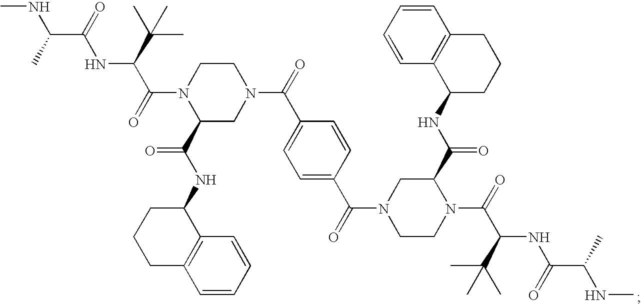 |
| 2 | 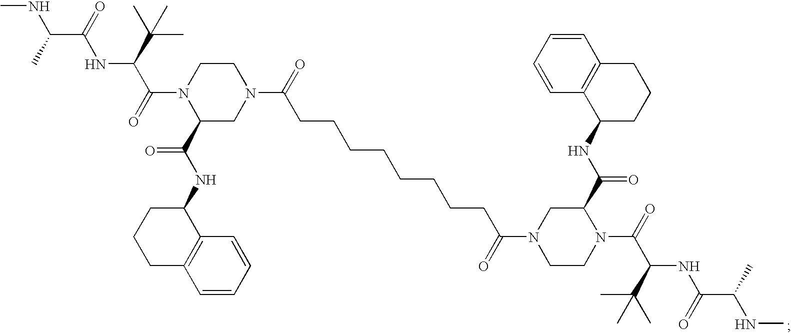 |
| 3 | 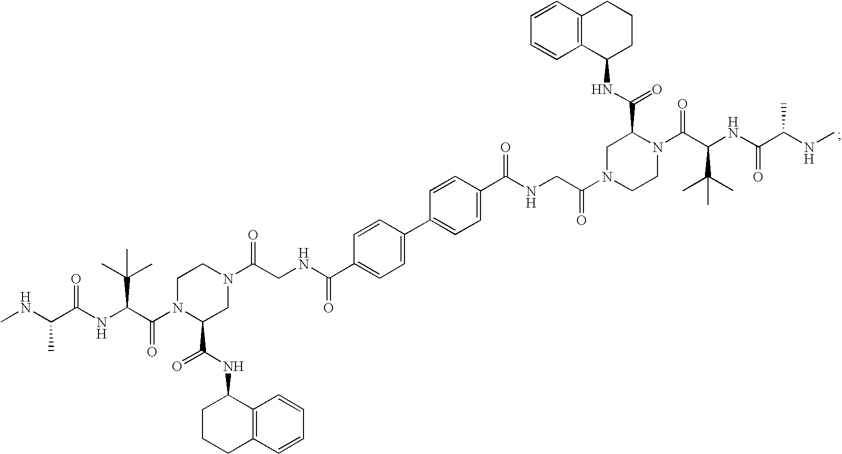 |
| 4 | 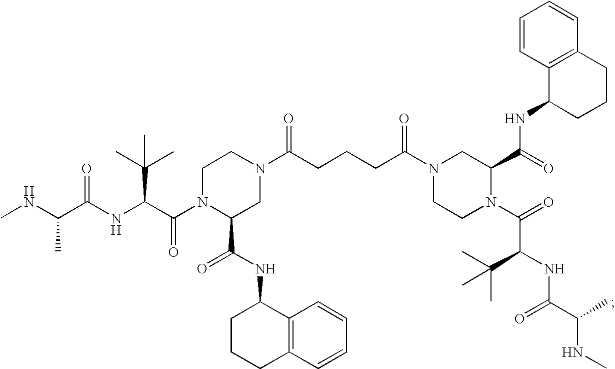 |
| 5 | 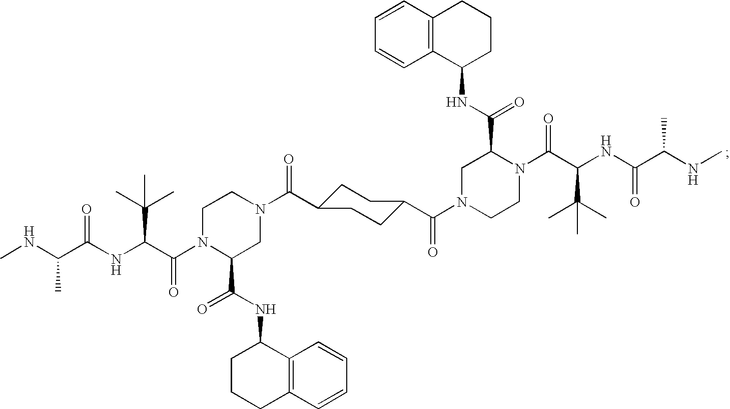 |
| 6 | 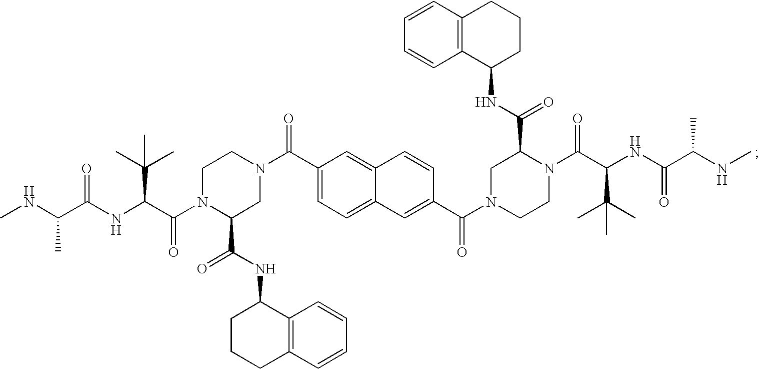 |
| 7 | 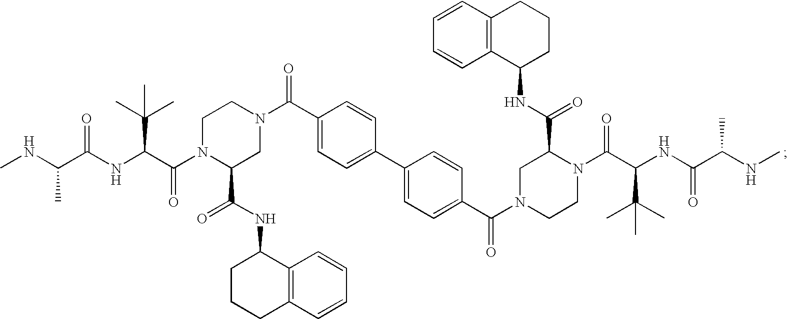 |
| 8 | 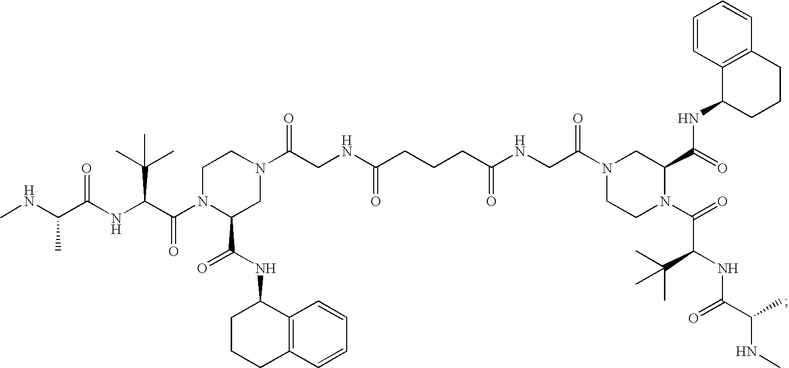 |
| 9 | 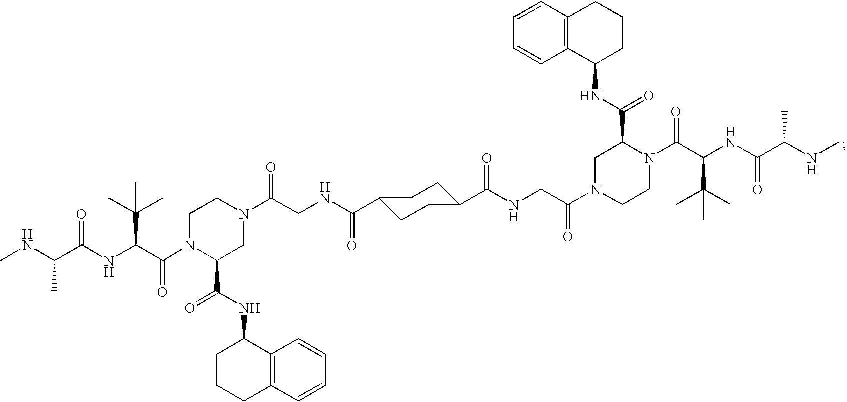 |
| 10 | 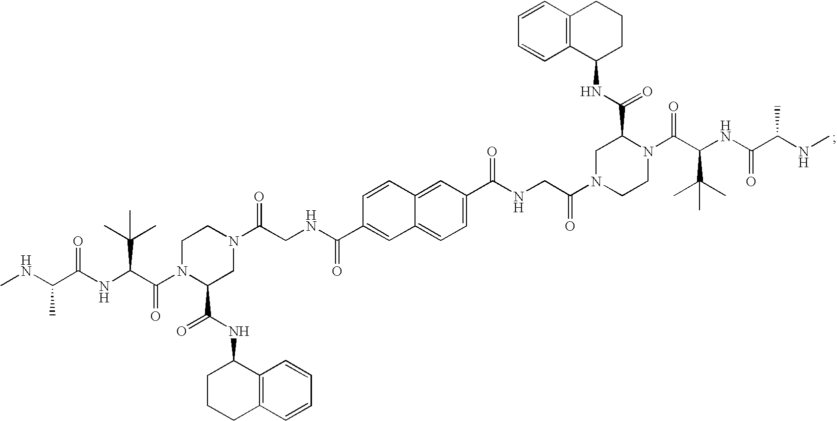 |
| 11 | 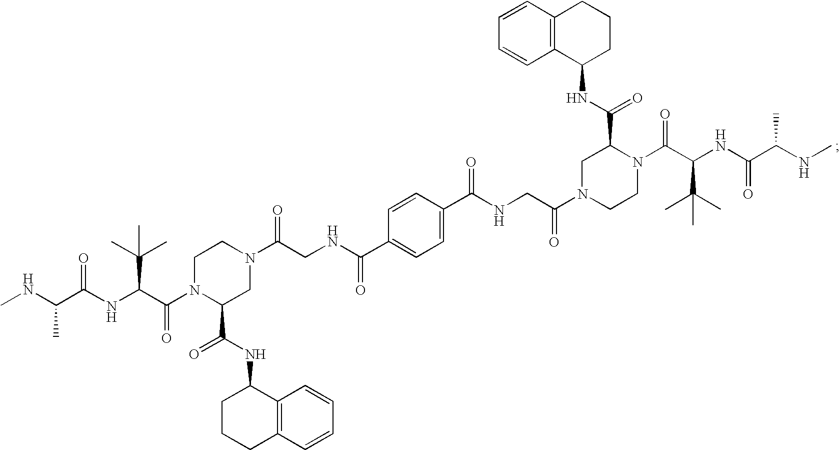 |
| 12 | 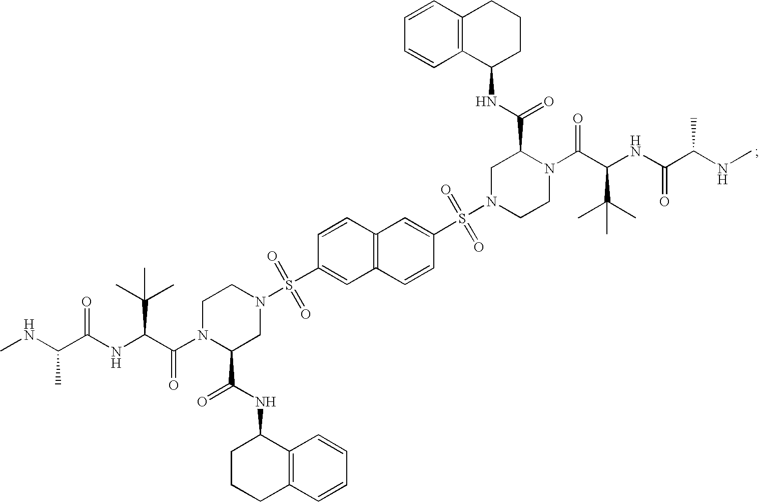 |
| 13 | 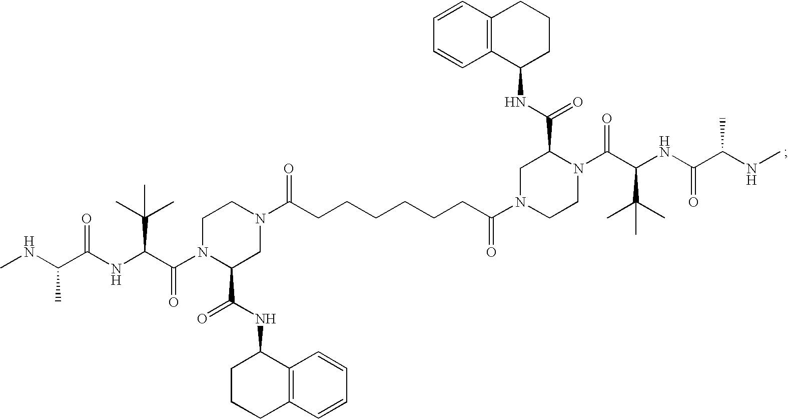 |
| 14 | 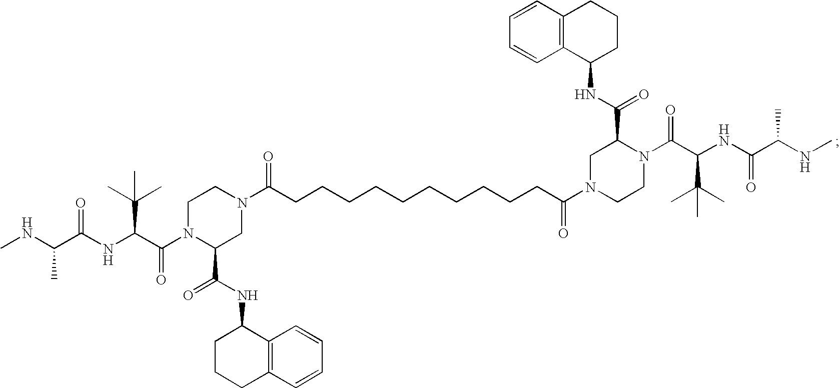 |
| 15 | 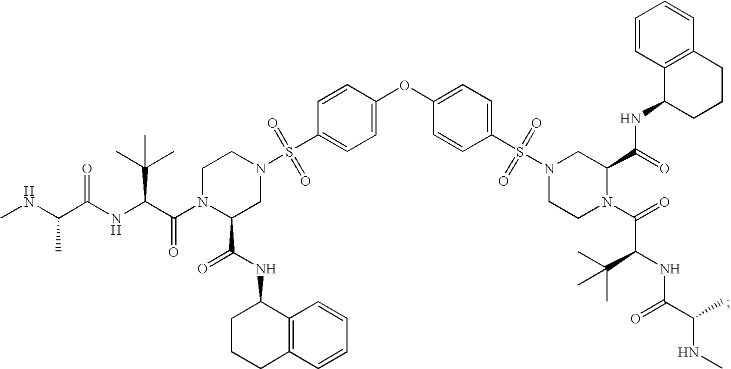 |
| 16 | 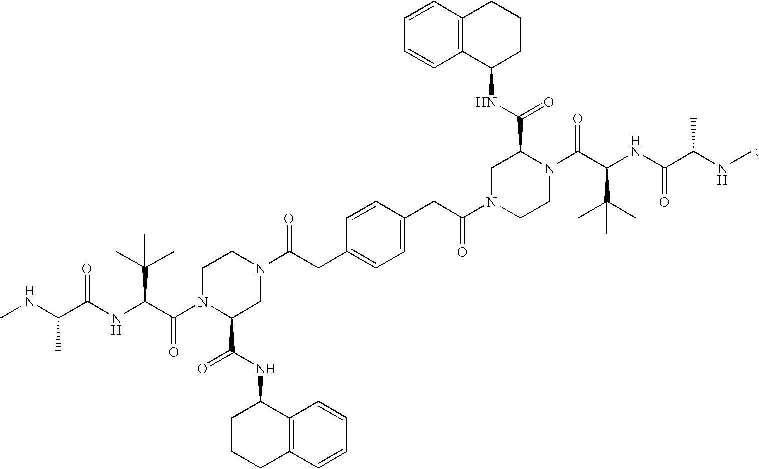 |
| 17 | 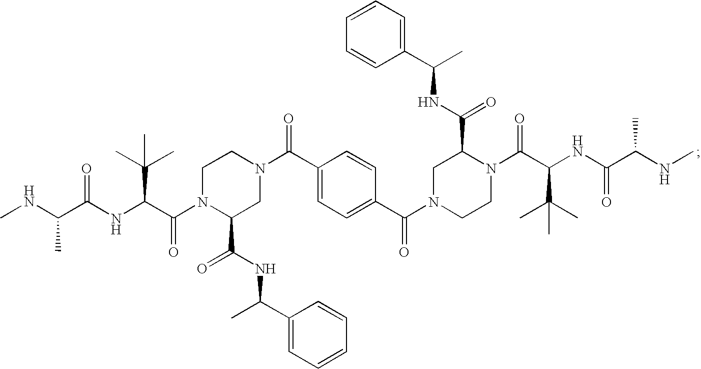 |
| 18 | 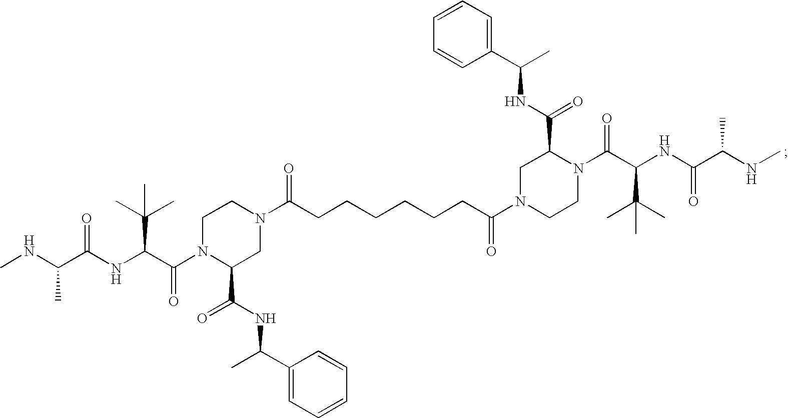 |
| 19 | 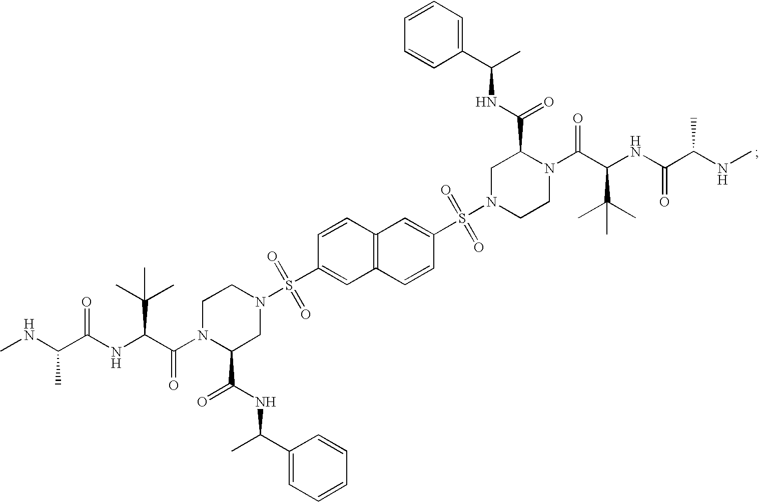 |
| 20 | 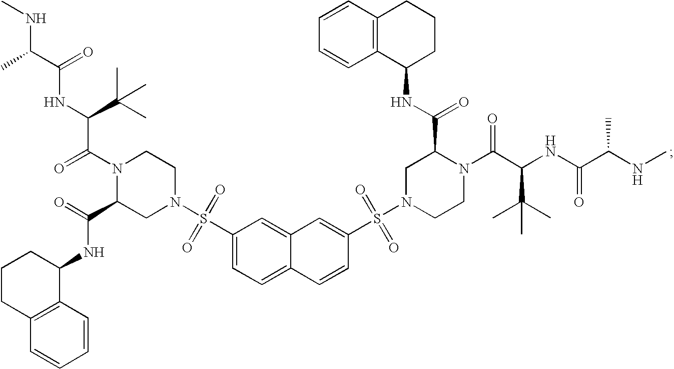 |
| 21 | 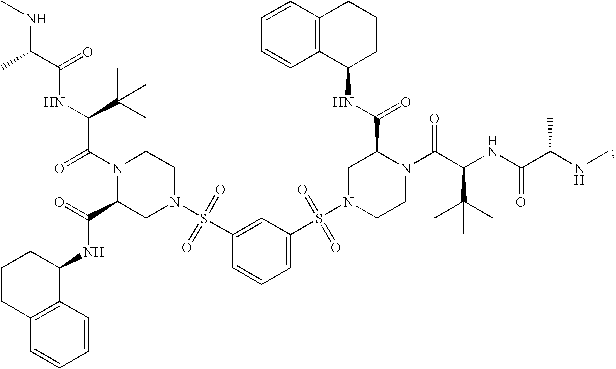 |
| 22 | 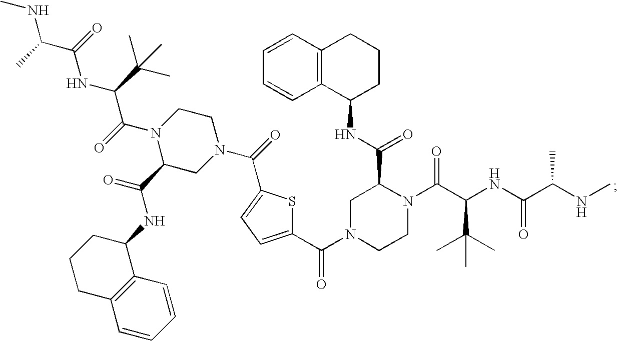 |
| 23 | 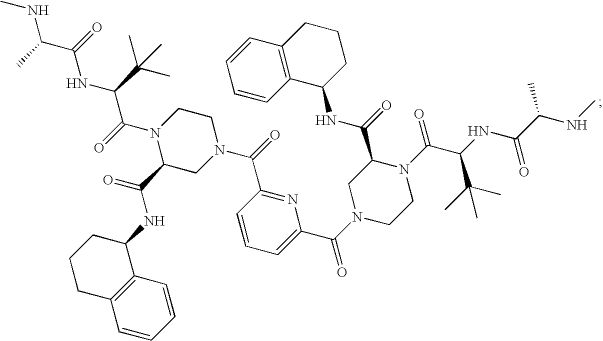 |
| 24 | 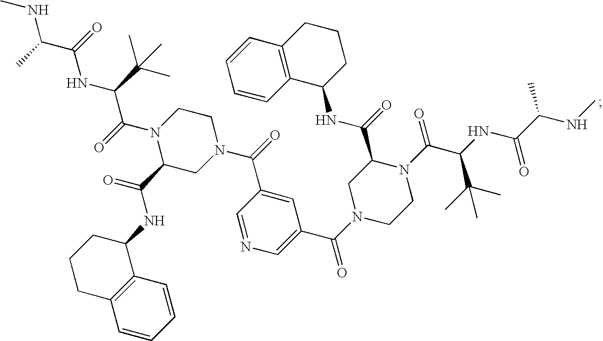 |
| 25 | 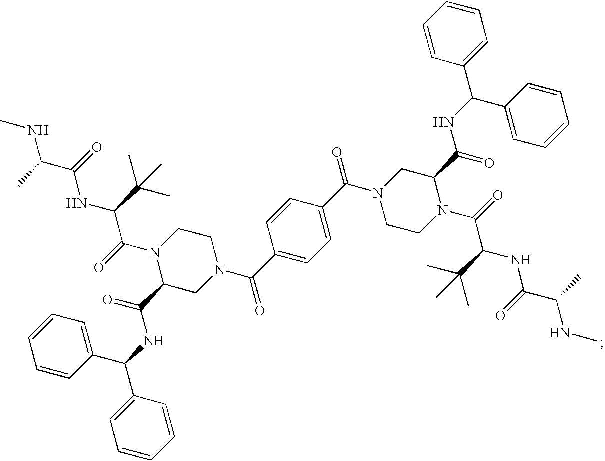 |
| 26 | 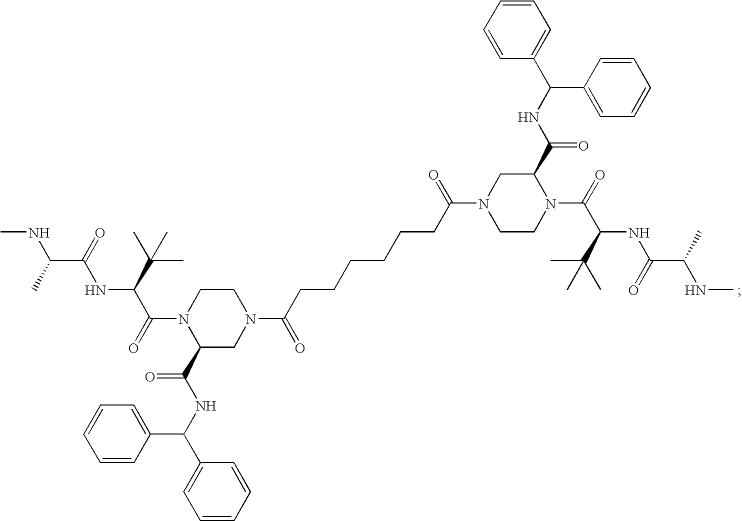 |
| 27 | 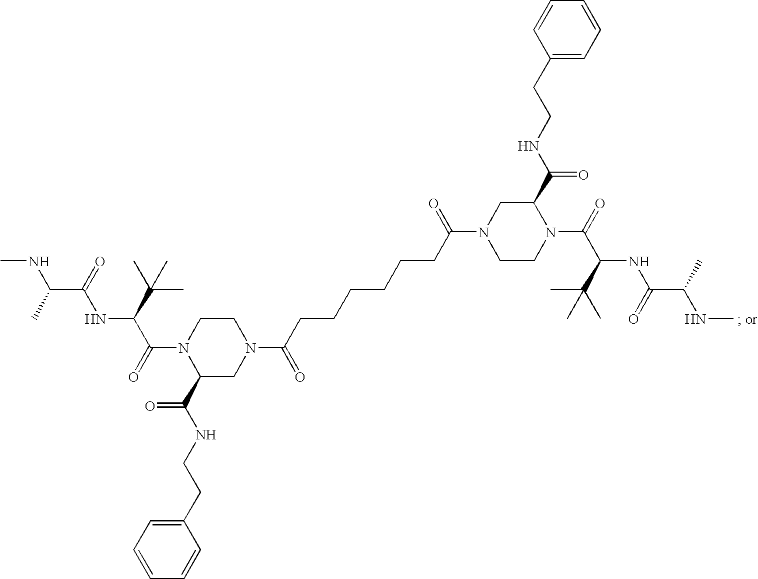 |
| 28 | 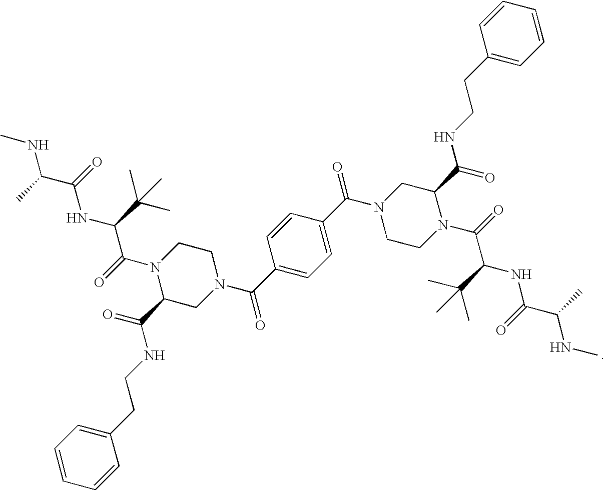 |

12. A process for producing compounds represented by Formula 1, described hereinabove, the process comprising:
a) coupling two intermediates represented by Formula 3: 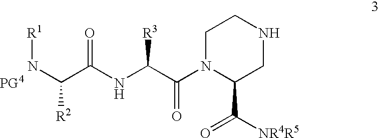
and LG-C(O)-L-C(O)-LG in a solvent with a base; and
b) removing the protecting groups so as to form compounds of Formula 12 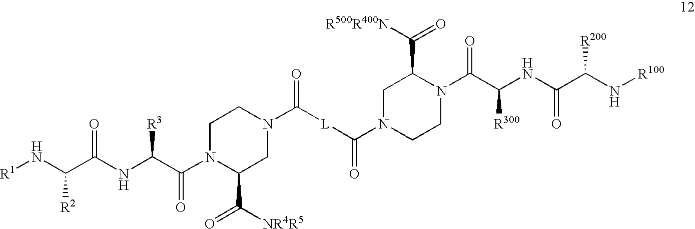
wherein L, R¹, R¹⁰⁰, R², R²⁰⁰, R³, R³⁰⁰, R⁴, R⁴⁰⁰, R⁵ and R⁵⁰⁰ are as defined in claim 1;
or
a) coupling two intermediates represented by Formula 3: 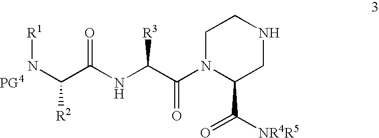
and LG-S(O)₂-L-S(O)₂-LG in a solvent with a base; and
b) removing the protecting groups so as to form compounds of Formula 13 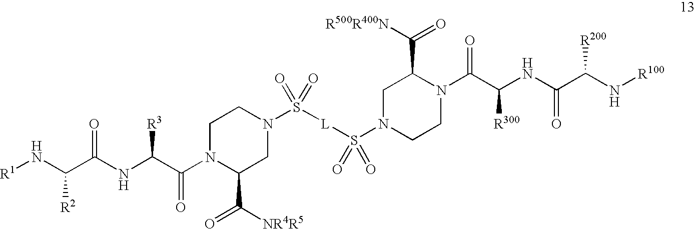
wherein L, R¹, R¹⁰⁰, R², R²⁰⁰, R³, R³⁰⁰, R⁴, R⁴⁰⁰, R⁵ and R⁵⁰⁰ are as defined in claim 1;
or
a) coupling two intermediates represented by Formula 11: 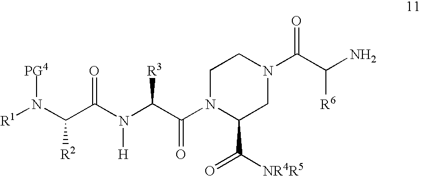
and LG(O)C-L-C(O)LG in a solvent with a base; and
b) removing the protecting groups so as to form compounds of Formula 14 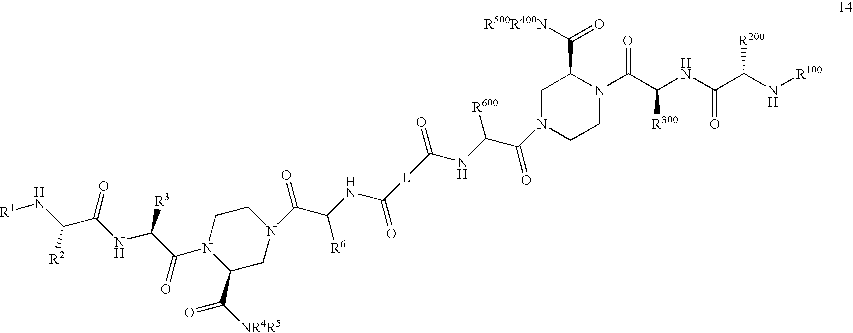
wherein L, R¹, R¹⁰⁰, R², R²⁰⁰, R³, R³⁰⁰, R⁴, R⁴⁰⁰, R⁵, R⁵⁰⁰, R⁶ and R⁶⁰⁰ are as defined in claim 1.

13. (canceled)

14. (canceled)

15. A method for the preparation of a pharmaceutically acceptable salt of a compound of claim 1 comprising treating a compound of Formula 1 with 1 to 2 equivalents of a pharmaceutically acceptable acid.

16. A pharmaceutical composition comprising a compound of claim 1 and a pharmaceutically acceptable carrier, diluent or excipient.

17. The composition of claim 16 further comprising one or more death receptor agonists.

18. The composition of claim 17, in which the death receptor agonist is an agonist of a TRAIL receptor.

19. The composition of claim 16 further comprising therapeutic agent that increases the response of one or more death receptor agonists.

20. The composition of claim 17, in which the death receptor agonist is a cytotoxic cytokine.

21. The composition of claim 20 in which the cytotoxic cytokine is an interferon.

22.-41. (canceled)

42. A method of enhancing apoptosis in a cell comprising contacting the cell with a compound of claim 1.

43. The method of claim 42, wherein the cell is a cancer cell.

44. The method of claim 42, wherein the cell is an immune cell.

45. The method of claim 42, wherein the cell is a neutrophil, monocyte, or T-cell.

46. The method of claim 42, wherein the cell is in a subject, and the cell is contacted with the compound of claim 1 by administering the compound of claim 1 to the subject.

47. The method of claim 46, further comprising administering to the subject a chemotherapeutic agent prior to, simultaneously with, or after administration of the compound of claim 1.

48. The method of claim 46, further comprising administering to the subject a death receptor agonist prior to, simultaneously with, or after administration of the compound of Formula I or salt thereof.

49. The method of claim 48, wherein the death receptor agonist is TRAIL or a TRAIL receptor antibody.

50. The method of claim 48, wherein the death receptor agonist is HGS-ETR1 or HGS-ETR2.

51. The method of claim 46, in which the death receptor agonist is administered in an amount that produces a synergistic effect.

52. The method of claim 42, in which the subject is a human.

53. The method of claim 52, wherein the subject is afflicted with a proliferative disease.

54. The method of claim 53, wherein the proliferative disease is cancer.

55. The method of claim 53, wherein the proliferative disease is an autoimmune disease or inflammatory disorder.
